(19) United States
(12) Patent Application Publication
(10) Pub. No.: US 2021/0380619 A1
(43) Pub. Date: Dec. 9, 2021

(54) Production method of cyclic compounds by olefin metathesis reaction and use of ruthenium catalysts in production of cyclic olefines by olefin metathesis reaction

(71) Applicant: UNIWERSYTET WARSZAWSKI, Warszawa (PL)

(72) Inventors: Karol GRELA, Warszawa (PL); Sylwia CZARNOCKA-SNIADALA, Warszawa (PL); Adrian SYTNICZUK, Minsk Mazowiecki (PL); Mariusz MILEWSKI, Nizdzin (PL); Mateusz URBAN, Katowice (PL); Lukasz BANACH, Bydgoszcz (PL); Michal DABROWSKI, Wysokie Mazowieckie (PL)

(73) Assignee: UNIWERSYTET WARSZAWSKI, Warszawa (PL)

(21) Appl. No.: 17/395,194

(22) Filed: Aug. 5, 2021

Related U.S. Application Data
(63) Continuation of application No. 16/609,209, filed on Oct. 28, 2019, now abandoned.

(30) Foreign Application Priority Data
Apr. 27, 2018 (PL) PL421462

(51) Int. Cl. C07F 15/00; B01J 31/16; C07C 6/04

(52) U.S. Cl. CPC C07F 15/0046; C07C 2531/16; C07C 6/04; B01J 31/1616

(57) ABSTRACT

The invention relates to a method for the preparation of cyclic compounds in the metathesis of olefins from acyclic dienes comprising terminal and/or non-terminal C═C double bonds; the invention also relates to the use of homogeneous ruthenium complexes and homogeneous ruthenium complexes deposited on a solid support as catalysts and/or pre-catalysts for the preparation of cyclic olefins in olefin metathesis reactions.

The invention relates to a method for preparation of cyclic compounds by the olefin metathesis reaction and the use of ruthenium catalysts for the preparation of cyclic olefins by olefin metathesis reactions The invention is applicable in the broadly understood organic synthesis using the ring closing metathesis (RCM) reaction.

### BACKGROUND ART

A number of ruthenium complexes are known in the art that allow obtaining of internal olefins [R. H. Grubbs (Ed.), A. G. Wenzel (Ed.), D. J. O'Leary (Ed.), E. Khosravi (Ed.), Handbook of Olefin Metathesis, 2nd edition, 3 Volumes, 2015, John Wiley & Sons, Inc. 1608 pages], among which one should note the 1st (Ru-I), 2nd (Ru-II) and 3rd (Ru-III) generation complexes and complexes comprising two, identical or different, N-heterocyclic carbene ligands (NHCs). In ruthenium complexes, the active, 14-electron catalyst form comprises a neutral ligand that is a phosphine or NHC [(i) Chem. Rev., 2010, 110, 1746-1787; (ii) Chem. Commun. 2014, 50, 10355-10375]. The complexes of the 2nd generation are the most universal and effective, namely so-called Grubbs (Gru-II), Hoveyda-Grubbs (Hov-II) and indenylidene (Ind-II) catalysts.

NHC ligands having various types, more or less complex steric hindrances and modified electronic properties are known from the literature [Chem. Rev., 2011, 111, 2705-2733] In general, these may be grouped into carbene imidazole and imidazolidine NHC ligands. Nitrogen atoms in carbene heterocycles of NHC ligands are mostly bound to aromatic substituents, e.g. 2,4,6-trimethylphen-1-yl (Mes) or 2,6-diisopropylphen-1-yl (Dipp). Literature reports on ruthenium complexes containing NHC ligands with N-alkyl substituents, but only scarcely. Therefore, our focus is on ruthenium compounds containing N-aryl substituents or a mixed system with N-aryl and N-aralkyl substituents in NHC ligands.

The olefin metathesis reaction can often shorten the path of synthesis, reducing the number of necessary steps and lowering the costs of the target product in preparation process, as it was in the course of the optimisation of macrocyclic HCV protease inhibitor known under the trade name Ciluprevir (BILN 2061) [(i) Org. Process Res. Dev., 2009, 13, 250-254; (ii) Org. Lett., 2008, 10, 1303-1306].

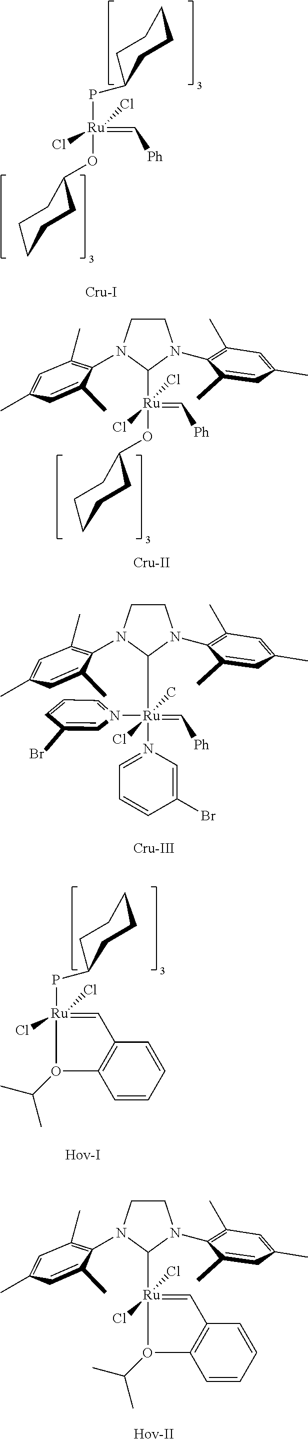

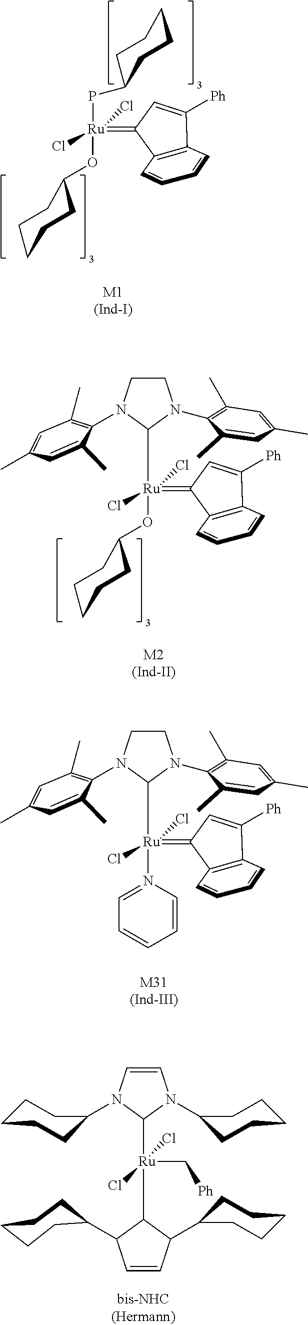

Despite multifarious advantages of the olefin metathesis reaction, its industrial application encounters many difficulties, especially in the preparation processes of macrocyclic rings in the RCM reaction. The first one is related to the thermodynamics of RCM reaction, as it is an equilibrium process [Chem. Rev., 2009, 109, 3783-3816]. The equilibrium of the metathesis reaction may be shifted towards the formation of products by removing gaseous products, e.g. ethylene, propene or butene, by conducting the reaction under reduced pressure or by using inert gas bubbling to flush out the gaseous products [WO2013048885A1].

The second major challenge of the RCM reaction is the lack of selectivity and the competition in the process of obtaining a cyclic product with polymers and oligomeric products [J. Am. Chem. Soc., 2007, 129, 1024-1025]. It is not a prevalent problem in case of the reactions yielding small cyclic olefins (5-6 membered rings), whereas RCM reactions yielding large macrocyclic rings (8-membered rings and larger) require high dilution (from C=20 mM to C=0.5 mM) to be used, which significantly hampers and clearly increases the cost of the process due to the large volumes of solvents [(i) Synthesis, 1997, 792-803; (ii) Synlett, 2002, 1302-1304; (iii) Eur. J. Org. Chem., 2004, 2053-2056]. Sometimes it is possible to inhibit processes that are competitive to the RCM reaction and restrict the formation of dimers and oligomers by forcing a favourable conformation/orientation of the substrate through the application of sterically expanded Lewis acids [Org. Lett., 2008, 10, 5613-5615].

The third problem of the metathesis reaction is the migration process of the C═C double bond both within the substrates and the target product [(i) J. Am. Chem. Soc., 2004, 126, 7414-7415; (ii) J. Am. Chem. Soc., 2007, 129, 7961-7968]. This phenomenon happens due to side reactions that are caused by ruthenium hydrides resulting from the decomposition of the olefin metathesis catalyst. This problem can be partly eliminated by using quinones or other ruthenium hydride inhibitors [J. Am. Chem. Soc., 2005, 127, 17160-17161].

The fourth hindrance of the use of olefin metathesis in an industrial scale results from the necessity to use a substantial volume of homogeneous ruthenium catalyst, often up to 20 mol % and more. There are already literature reports known where an olefin metathesis catalyst was used in an amount of up to 1 ppm [(i) Adv. Synth. Catal., 2002, 344, 671-677; (ii) Angew. Chem. Int. Ed., 2017, 56, 981-986]. However, it should be emphasised that the substrates of these conversions are simple (non-functionalised) olefins, possibly their esters in cross-metathesis or RCM reactions yielding small rings, but not harder-forming macrocyclic products.

A valuable group of compounds classified as components of musk (widely used in the perfume and scents industry) are macrocyclic compounds such as Cyclopentadecanolide, Civetone, Muscone and Astrotone. Attempts to selectively and efficiently prepare these compounds are conducted from the early stages of the development of olefin metathesis reactions [(i) Tetrahedron Lett., 1980, 21, 1715-1718; (ii) Tetrahedron Lett., 1980, 21, 2955-2958]. The latest literature reports mainly concern the improvement of diastereoselectivity (E/Z) of the C═C double bond produced during macrocyclisation in the RCM reaction [(i) J. Am. Chem. Soc., 2013, 135, 94-97; (ii) Nature, 2017, 541, 380-385; (iii) WO2012167171A2; (iv) J. Am. Chem. Soc., 2017, 139, 1532-1537].

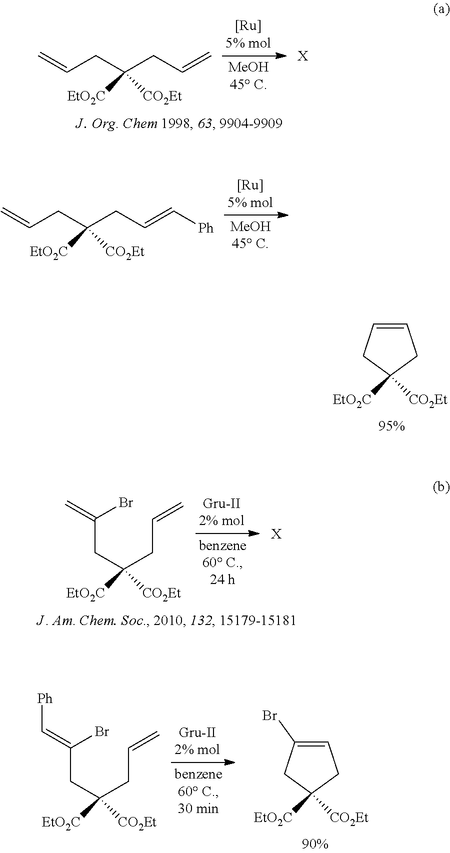

On the other hand, a few literature reports are known in which use of an additional substituent (e.g. phenyl) in the vicinal position increases the efficiency of the process, or even allows for obtaining the desired product. Such a strategy has been used by Grubbs et al. to conduct metathesis reactions in polar solvents, alcohols and water-alcohol mixtures [J. Org. Chem., 1998, 63, 9904-9909]. Dorta et al., in turn, used such a strategy to conduct the RCM reaction of vinyl bromides [J. Am. Chem. Soc., 2010, 132, 15179-15181].

### Technical Problem

In the summary of prior art, similar technical problems regarding the synthesis of macrocyclic compounds by the RCM reaction appear in further reports. These include: (a) the equilibrium nature of the olefin metathesis reaction—thermodynamic products are yielded as a result of shifting the equilibrium of the reaction towards the desired products by removing one of the products from the reaction environment; (b) high dilution of reagents in the reaction environment (low concentration), which requires the use of large volumes of solvents (which significantly increases the cost of the process and also makes it necessary to use larger, and therefore more expensive reactors); (c) decomposition of the ruthenium reaction catalyst and the resulting migration of the double bond in reaction substrates and products, reducing the selectivity of the process; (d) high load of homogeneous olefin metathesis catalysts based on costly ruthenium.

### Solution to the Problem

During conducting meticulous optimisation of the conditions of the diene macrocyclization process by RCM reaction, it was surprisingly found that use of substrates with suitably substituted C═C double bonds, use of a low load of a suitable catalyst, addition of adjuvant reagents, use of a suitable solvent and reduction of pressure leads to obtaining the desired macrocyclization product with high yield and selectivity.

### Disclosure of the Essence of the Invention

This invention relates to the use of a compound of formula 1,

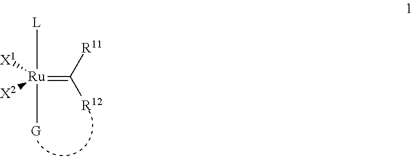

wherein:

X¹ and X² are each independently an anionic ligand selected from such as halogen atoms, —CN, —SCN, —OR′, —SR′, —O(C═O)R′, —O(SO₂)R′, —OSi(R′)₃ group, wherein R′ is C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom; R¹¹, R¹² are each independently a hydrogen atom, a halogen atom, optionally substituted C₁-C₂₅ alkyl, optionally substituted C₁-C₂₅ perfluoralkyl, optionally substituted C₂-C₂₅ alkene, optionally substituted C₃-C₇ cycloalkyl, optionally substituted C₂-C₂₅ alkenyl, optionally substituted C₃-C₂₅ cycloalkenyl, optionally substituted C₂-C₂₅ alkinyl, optionally substituted C₃-C₂₅ cycloalkinyl, optionally substituted C₁-C₂₅ alkoxy, optionally substituted C₅-C₂₄ aryloxy, optionally substituted C₅-C₂₀ heteroaryloxy, optionally substituted C₅-C₂₄ aryl, optionally substituted C₅-C₂₀ heteroaryl, optionally substituted C₇-C₂₄ aralkyl, optionally substituted C₅-C₂₄ perfluoroaryl, optionally substituted 3-12-membered heterocycle;

wherein the substituents R¹¹ and R¹² may be interconnected to form a ring selected from the group consisting of C₃-C₇ cycloalkyl, C₃-C₂₅ cycloalkenyl, C₃-C₂₅ cycloalkinyl, C₅-C₂₄ aryl, C₅-C₂₀ heteroaryl, C₅-C₂₄ perfluoroaryl, 3-12-membered heterocycle, each of which may be substituted with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle, in which case the dotted line between G and R¹² is not a chemical bond;

wherein the substituents R¹¹ i R¹² preferably are a hydrogen atom or aryl independently substituted with the following groups: alkoxy (—OR′), sulfide (—SR′), sulfoxide (—S(O)R′), sulfonium (—S⁺R′₂), sulphonic (—SO₂R′), sulfonamide (—SO₂NR′₂), amino (—NR′₂), ammonium (—NR′₃), nitro (—NO₂), cyano (—CN), phosphonium (—P(O)(OR′)₂), phosphinium (—P(O)R′(OR′)), phosphonous (—P(OR′)₂), phosphine (—PR′₂), phosphine oxides (—P(O)R′₂), phosphonium (—P⁺R′₃), carboxy (—COOH), ester (—COOR′), amide (—CONR′₂), amide (—NR′COR″), formyl (—CHO), ketone (—COR′), in which groups R′ is C₁-C₅ alkyl, C₁-C₅ perfluoroalkyl, C₅-C₂₄ aryl, C₇-C₂₄ aralkyl, C₅-C₂₄ perfluoroaryl;

L is selected from such as:

a)

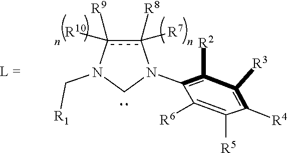

wherein:

R¹ is a heteroaryl group;

R², R³, R⁴, R⁵, R⁶ are each independently a hydrogen atom, a C₁-C₂₅ alkyl group, a C₁-C₂₅ alkoxy group or a C₂-C₂₅ alkenyl group, wherein the substituents R², R³, R⁴, R⁵, R⁶ may be interconnected to form a substituted or unsubstituted cyclic C₄-C₁₀ or polycyclic C₄-C₁₂ system;

R⁷, R⁸, R⁹, i R¹⁰ are each independently a hydrogen atom or a C₁-C₂₅ alkyl group, or R⁷ and/or R⁸ can be connected with R⁹ and/or R¹⁰ to form a cyclic system;

n is 0 or 1;

b)

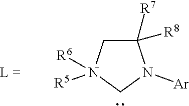

wherein:

Ar is an aryl group which is substituted with hydrogen atoms or is optionally substituted with at least one of the following groups: C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, C₇-C₂₄ aralkyl, C₅-C₂₄ perfluoroaryl, or a halogen atom;

R⁵, R⁶, R⁷ and R⁸ are each independently a hydrogen atom or one of the following groups: C₁-C₂₅ alkyl, C₃-C₁₂cycloalkyl, C₁-C₅ perfluoroalkyl, C₂-C₁₂ alkenyl, C₅-C₂₀ aryl, C₅-C₂₄ aryloxy, C₂-C₂₀ heterocyclo, C₄-C₂₀ heteroarylo, C₅-C₂₀ heteroaryloxy, C₇-C₂₄ aralkyl, C₅-C₂₄ perfluoroaryl, which are optionally substituted with at least one C₁-C₁₂ alkyl, C₁-C₁₂ perfluoroalkyl, C₁-C₁₂ alkoxy, C₅-C₂₄ aryloxy, C₄-C₂₀ heteroaryl, C₅-C₂₀ heteroaryloxy, or a halogen atom; moreover, R⁵ and R⁶ and/or R⁷ i R³ may be interconnected to form a cyclic system;

c)

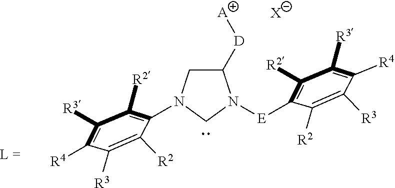

wherein:

the combination A⁺X⁻ and D is a hydrogen atom, or

A is independently a substituent containing a tertiary amine group or a quaternary ammonium group, which may be a N(R¹)(R²) or N(R¹)(R²)(R³) group, wherein R¹, R² and R³ are each independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl; alternatively, A is one of the following groups: C₁-C₂₅ cycloaminoalkyl, C₁-C₂₅ cyclodiaminoalkyl, C₁-C₂₅ cyclotriaminoalkyl, C₁-C₂₅ cyclotetraaminoalkyl, C₁-C₂₅ cycloaminoammonioalkyl, wherein the at least one nitrogen atom is independently substituted with at least one R¹ group, wherein R¹ is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl so that at least one nitrogen atom in the ring forms a quaternary ammonium group;

X is independently a halogen atom, or CF₃SO₃⁻, BF₄⁻, PF₆⁻, and ClO₄⁻;

D is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl, or C₂-C₂₅ alkenyl, C₁-C₂₅ α,ω-dialkoxy, (CH₂CH₂O)ₙ, polyether, where n comprises from 1 to 25, a C₁-C₂₅ thioalkyl group, a C₁-C₂₅ α,ω-dithioalkyl group, a C₁-C₂₅ α,ω-diheteroalkyl group, a C₁-C₂₅ aminoalkyl group;

E is a single bond or independently a C₁-C₂₅ alkyl group, a C₁-C₁₂ perfluoroalkyl group, a C₃-C₇ cycloalkyl group, a C₁-C₂₅ alkoxy group;

X¹ and X² are each independently an anion ligand selected from such as halogen atoms;

R², R^{2′}, R³, R^{3′} i R⁴ are each independently a hydrogen atom, a halogen atom, a C₁-C₂₅ alkyl group, a C₃-C₇ cycloalkyl group, a C₁-C₂₅ alkoxy group, a C₅-C₂₄ perfluoroaryl group, a C₅-C₂₀ heteroaryl group or a C₂-C₂₅ alkenyl group, wherein the substituents R², R^{2′}, R³, R^{3′} and R⁴ may be interconnected to form a substituted or unsubstituted cyclic C₄-C₁₀ or polycyclic C₄-C₁₂ system;

and

G is selected from the substituents L listed above or G is a heteroatom selected from the group comprising an oxygen, nitrogen, sulphur, phosphorus, fluorine, chlorine, bromine and iodine atom, optionally substituted with a group selected from such as hydrogen atom, C₁-C₂₅ alkyl, C₁-C₂₅ perfluoroalkyl, C₃-C₇ cycloalkyl, C₅-C₂₄ aryl, C₅-C₂₄ perfluoroaryl, C₅-C₂₀ heteroaryl, C₇-C₂₄ aralkyl, 3-12 membered heterocycle, from the following groups: —COR′ acyl, (—CN) cyano, (—COOH) carboxy, (—COOR′) ester, (—CONR′₂) amide, (—SO₂R′) sulfonic, (—CHO) formyl, (—SO₂NR′₂) sulfonamide, (—COR′) ketone, wherein the R′ group is C₁-C₅ alkyl, C₁-C₅ perfluoroalkyl, C₅-C₂₄ aryl, C₅-C₂₄ perfluoroaryl, C₇-C₂₄ aralkyl, in which case the dotted line is a direct bond between the heteroatom and the R¹² substituent, in the form of an aryl optionally substituted by 1-4 substituents independently selected from the group comprising a hydrogen atom, a halogen atom, C₁-C₂₅ alkyl, C₁-C₂₅ perfluoroalkyl, C₂-C₂₅ alken, C₃-C₇ cycloalkyl, C₂-C₂₅ alkenyl, C₃-C₂₅ cycloalkenyl, C₂-C₂₅ alkinyl, C₃-C₂₅ cycloalkinyl, C₅-C₂₄ aryl, C₇-C₂₄ aralkyl, C₅-C₂₄ perfluoroaryl, C₅-C₂₀ heteroaryl, 3-12-membered heterocycle, one of the following groups: (—OR′) alkoxy, (—SR′) sulfide, (—NO₂) nitro, (—CN) cyano, (—COOH) carboxy, (—COOR′) ester, (—CONR′₂) amide, (—CONR′COR′) imide, (—NR′₂) amino, (—NR′₃) ammonium, (—NR′COR′) amide, (—NR′SO₂R′), sulfonamide (—SO₂R′), sulfonic (—CHO), formyl (—SO₂NR′₂), sulfonamide (—COR′), ketone, in which groups R′ is as follows: C₁-C₅ alkyl, C₁-C₅ perfluoroalkyl, C₅-C₂₄ aryl, C₅-C₂₄ perfluoroaryl, C₇-C₂₄ aralkyl; in olefin metathesis reactions involving contacting olefins with the formula Dx

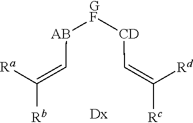

wherein:

AB and CD are each independently a group selected from such as a hydrogen atom, C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be optionally substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

R^{a}, R^{b}, R^{c} i R^{d} are each independently C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

or olefins Dy with the formula

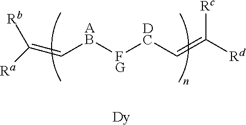

wherein:

AB and CD are each independently a group selected from such as C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be optionally substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

GF is an ether (—O—), ester (—C(O)O—), carbonyl (—C(O)—), amido (—C(O)NR—), malonate (—C(COOR)₂—) group, wherein R is a independently hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

R^{a}, R^{b}, R^{c} i R^{d} are each independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

with the compound with the formula 1 in the presence of other additives, wherein the main product is obtained in the form of at least one cyclic compound Mx comprising at least one non-terminal double C═C bond,

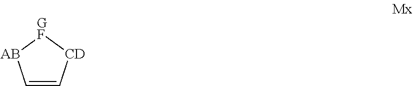

wherein:

AB and CD are each independently a group selected from such as C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom.

Preferably, compound 1 is compound 1a

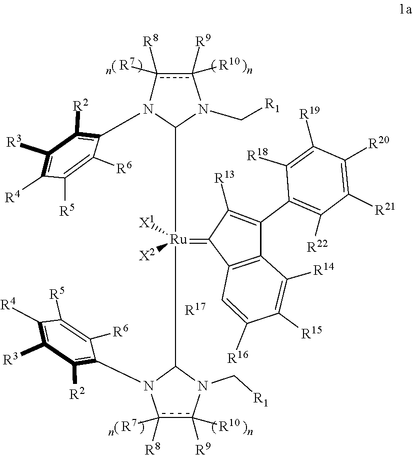

wherein:

X¹, X² are a halogen atom;

R¹ is a heteroaryl selected from the group comprising furan, thiophene, benzothiophene, benzofuran;

R², R³, R⁴, R⁵, R⁶ are each independently a hydrogen atom, methyl, isopropyl, a halogen atom;

R⁷, R⁸, R⁹, R¹⁰ are each independently a hydrogen atom or methyl;

n is 0 or 1;

R¹³, R¹⁴, R¹⁵, R¹⁶, R¹⁷, R¹⁸, R¹⁹, R²⁰, R²¹ and R²² are each independently a hydrogen atom, a halogen atom, one of the following groups: C₁-C₂₅ alkyl, C₁-C₂₅ alkylamino, C₁-C₂₅ alkylammonium, C₁-C₂₅ perfluoroalkyl, C₂-C₂₅ alkenyl, C₃-C₇ cycloalkyl, C₃-C₂₅ cycloalkenyl, C₂-C₂₅ alkynyl, C₃-C₂₅ cycloalkynyl, C₁-C₂₅ alkoxy, C₅-C₂₄ aryl, C₅-C₂₀ heteroaryl, C₃-C₁₂ heterocycle, 3-12-membered heterocycle, a sulfide (—SR′), ester (—COOR′), amido (—CONR′₂), sulfonic (—SO₂R′), sulfonamide (—SO₂NR′₂) or ketone (—COR′), group, wherein R′ is a C₁-C₅ alkyl, C₁-C₅ perfluoroalkyl, C₅-C₂₅ aryl or C₅-C₂₅ perfluoroaryl group.

Preferably, compound 1 is compound 1b

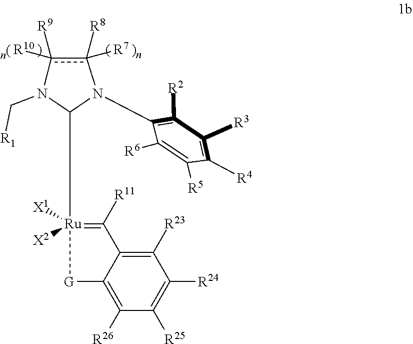

wherein:

X¹, X² are a halogen atom;

R¹ is a heteroaryl selected from the group comprising furan, thiophene, benzothiophene, benzofuran;

R², R³, R⁴, R⁵, R⁶ are each independently a hydrogen atom, methyl, isopropyl, a halogen atom;

R⁷, R⁸, R⁹, R¹⁰ are each independently a hydrogen atom or methyl;

n is 0 or 1;

R¹¹ is a hydrogen atom;

R²³, R²⁴, R²⁵, R²⁶ are each independently a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₅-C₂₄ aryl, C₇-C₂₄ aralkyl, C₅-C₂₄ perfluoroaryl, C₅-C₂₀ heteroaryl, 3-12 membered heterocycle, one of the following groups: alkoxy (—OR′), sulfide (—SR′), nitro (—NO₂), cyano (—CN), carboxy (—COOH), ester (—COOR′), amido (—CONR′₂), imido (—CONR′COR′), amino (—NR′₂), ammonium (—N⁺R′₃), amide (—NR′COR′), sulfonamide (—NR′SO₂R′), sulfonate (—SO₂R′), formyl (—CHO), sulfonamide (—SO₂NR′₂), ketone (—COR′), in which R′ groups are as follows: C₁-C₅ alkyl, C₁-C₅ perfluoroalkyl, C₅-C₂₄ aryl, C₅-C₂₄ perfluoroaryl, C₇-C₂₄ aralkyl, wherein R²³, R²⁴, R²⁵, R²⁶ are preferably a hydrogen atom;

G is a halogen atom or a substituent selected from the group comprising OR′, SR′, S(O)R′, S(O)₂R′N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;

Preferably, compound 1 is compound 1c

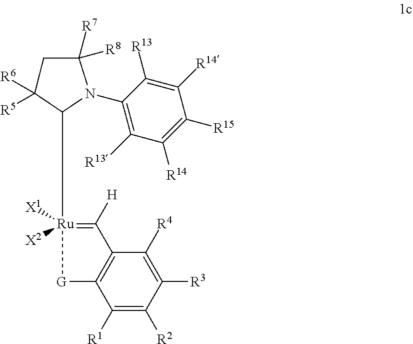

X¹, X² are a halogen atom;

G is a halogen atom or a substituent selected from the group OR′, SR′, S(O)R′, S(O)₂R′ N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;

R¹, R², R³, R⁴ are each independently a hydrogen atom, a sulfoxide group (—S(O)R′), a sulfonamide group (—SO₂NR′₂), a nitro group (—NO₂), an ester group (—COOR′), a ketone group (—COR′), a —NC(O)R′ ammonium group, a (—OMe) alkoxy group, in which groups R′ is C₁-C₅ alkyl, C₁-C₅ perfluoroalkyl, C₅-C₂₄ aryl, C₇-C₂₄ aralkyl, C₅-C₂₄ perfluoroaryl;

R⁵, R⁶, R⁷, i R⁸ are each independently a hydrogen atom or a C₁-C₂₅ alkyl group, a C₅-C₂₀ aryl group, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom; moreover, R⁵ and R⁶ and/or R⁷ and R⁸ may be interconnected to form a cyclic system.

R¹³ and R^{13′} are each independently methyl or ethyl;

R¹⁴, R^{14′}, R¹⁵ are each independently a hydrogen atom, a C₁-C₂₅ alkyl group.

Preferably, compound 1 is compound 1d

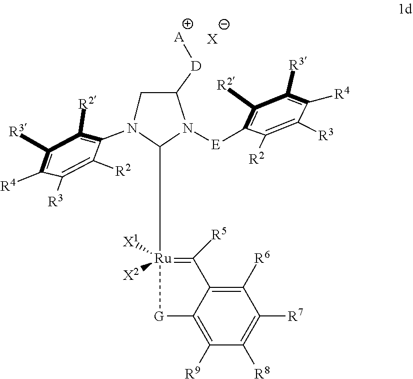

wherein:

the combination A⁺X⁻ and D is a hydrogen atom, or

A is independently a substituent containing a tertiary amine group or a quaternary ammonium group, which may be a N(R¹)(R²) or N(R¹)(R²)(R³) group, wherein R¹, R² and R³ are each independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl; alternatively, A is one of the following groups: C₁-C₂₅ cycloaminoalkyl, C₁-C₂₅ cyclodiaminoalkyl, C₁-C₂₅ cyclotriaminoalkyl, C₁-C₂₅ cyclotetraaminoalkyl, C₁-C₂₅ cycloaminoammonioalkyl, wherein the at least one nitrogen atom is independently substituted with at least one R¹ group, wherein R¹ is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl so that at least one nitrogen atom in the ring forms a quaternary ammonium group;

X is independently a halogen atom, or CF₃SO₃⁻, BF₄⁻, PF₆⁻, and ClO₄⁻;

D is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl, or C₂-C₂₅ alkenyl, C₁-C₂₅ α,ω-dialkoxy, (CH₂CH₂O)ₙ, polyether, where n comprises from 1 to 25, a C₁-C₂₅ thioalkyl group, a C₁-C₂₅ α,ω-dithioalkyl group, a C₁-C₂₅ α,ω-diheteroalkyl group, a C₁-C₂₅ aminoalkyl group;

E is independently a C₁-C₂₅ alkyl group, a C₁-C₁₂ perfluoroalkyl group, a C₃-C₇ cycloalkyl group, a C₁-C₂₅ alkoxy group, or a single bond;

X¹ and X² are each independently an anion ligand selected from halogen anions;

R¹ is independently a C₁-C₂₅ alkyl group, a C₃-C₇ cycloalkyl group, a C₅-C₂₄ aryl group, a C₅-C₂₀ heteroaryl group;

R², R^{2′}, R³, R^{3′} i R⁴ are each independently a hydrogen atom, a halogen atom, a C₁-C₂₅ alkyl group, a C₃-C₇ cycloalkyl group, a C₁-C₂₅ alkoxy group, a C₅-C₂₄ perfluoroaryl group, a C₅-C₂₀ heteroaryl group or a C₂-C₂₅ alkenyl group, wherein the substituents R², R^{2′}, R³, R^{3′} and R⁴ may be interconnected to form a substituted or unsubstituted cyclic C₄-C₁₀ or polycyclic C₄-C₁₂ system;

R⁵ is a hydrogen atom, a C₁-C₂₅ alkyl group, a C₃-C₇ cycloalkyl group;

R⁶, R⁷, R⁸, R⁹ are each independently a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₅-C₂₄ aryl, C₇-C₂₄ aralkyl, C₅-C₂₄ perfluoroaryl, C₅-C₂₀ heteroaryl, 3-12 membered heterocycle, one of the following groups: alkoxy (—OR′), sulfide (—SR′), nitro (—NO₂), cyano (—CN), carboxy (—COOH), ester (—COOR′), amido (—CONR′₂), imido (—CONR′COR′), amino (—NR′₂), ammonium (—NR′₃), amide (—NR′COR′), sulfonamide (—NR′SO₂R′), sulfonate (—SO₂R′), formyl (—CHO), sulfonamide (—SO₂NR′₂), ketone (—COR′), in which R′ groups are as follows: C₁-C₅ alkyl, C₁-C₅ perfluoroalkyl, C₅-C₂₄ aryl, C₅-C₂₄ perfluoroaryl, C₇-C₂₄ aralkyl, wherein R²³, R²⁴, R²⁵, R²⁶ are preferably a hydrogen atom;

G is a halogen atom or a substituent selected from the group OR′, SR′, S(O)R′, S(O)₂R′ N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;

Preferably, compound 1 is compound If

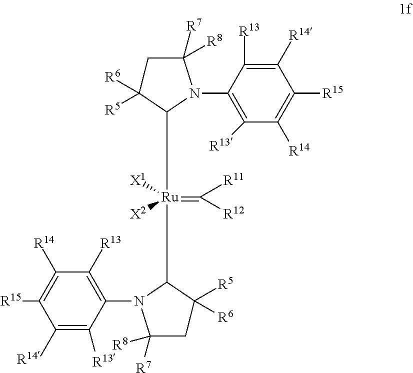

wherein:

X¹ and X² are each independently an anionic ligand selected from such as halogen atoms, —CN, —SCN, —OR′, —SR′, —O(C═O)R′, —O(SO₂)R′, —OSi(R′)₃ group, wherein R′ is C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy, or a halogen atom;

R⁵, R⁶, R⁷ and R⁸ are each independently a hydrogen atom or a C₁-C₂₅ alkyl group, R⁷ and/or R⁸ may be interconnected with R⁹ and/or R¹⁰ to form a cyclic system, they also may be independently the following groups: C₁-C₁₂ alkyl, C₃-C₁₂ cycloalkyl, C₂-C₁₂ alkenyl, C₅-C₂₀ aryl, C₁-C₅ perfluoralkyl, C₇-C₂₄ aralkyl, C₅-C₂₄ perfluoroaryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

R¹¹, R¹² are each independently a hydrogen atom, a halogen atom, optionally substituted C₁-C₂₅ alkyl, optionally substituted C₁-C₂₅ perfluoralkyl, optionally substituted C₂-C₂₅ alkene, optionally substituted C₃-C₇ cycloalkyl, optionally substituted C₂-C₂₅ alkenyl, optionally substituted C₃-C₂₅ cycloalkenyl, optionally substituted C₂-C₂₅ alkinyl, optionally substituted C₃-C₂₅ cycloalkinyl, optionally substituted C₁-C₂₅ alkoxy, optionally substituted C₅-C₂₄ aryloxy, optionally substituted C₅-C₂₀ heteroaryloxy, optionally substituted C₅-C₂₄ aryl, optionally substituted C₅-C₂₀ heteroaryl, optionally substituted C₇-C₂₄ aralkyl, optionally substituted C₅-C₂₄ perfluoroaryl, optionally substituted 3-12-membered heterocycle;

wherein the substituents R¹¹ and R¹² may be interconnected to form a ring selected from the group consisting of C₃-C₇ cycloalkyl, C₃-C₂₅ cycloalkenyl, C₃-C₂₅ cycloalkinyl, C₅-C₂₄ aryl, C₅-C₂₀ heteroaryl, C₅-C₂₄ perfluoroaryl, 3-12-membered heterocycle, each of which may be substituted with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

R¹³ and R^{13′} are each independently methyl or ethyl;

R¹⁴, R^{14′}, R¹⁵ are each independently a hydrogen atom, a C₁-C₂₅ alkyl group.

Preferably, compound 1 is represented by a formula with a structure selected from such as:

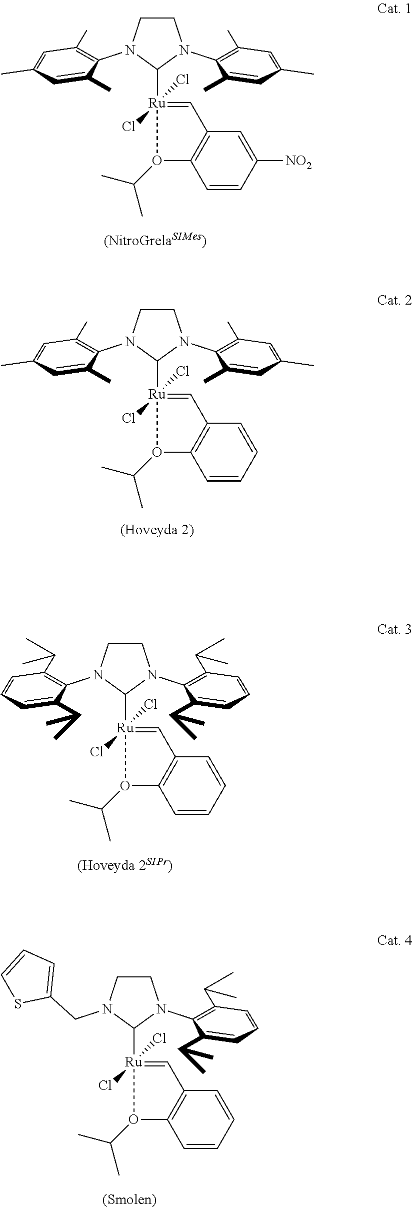

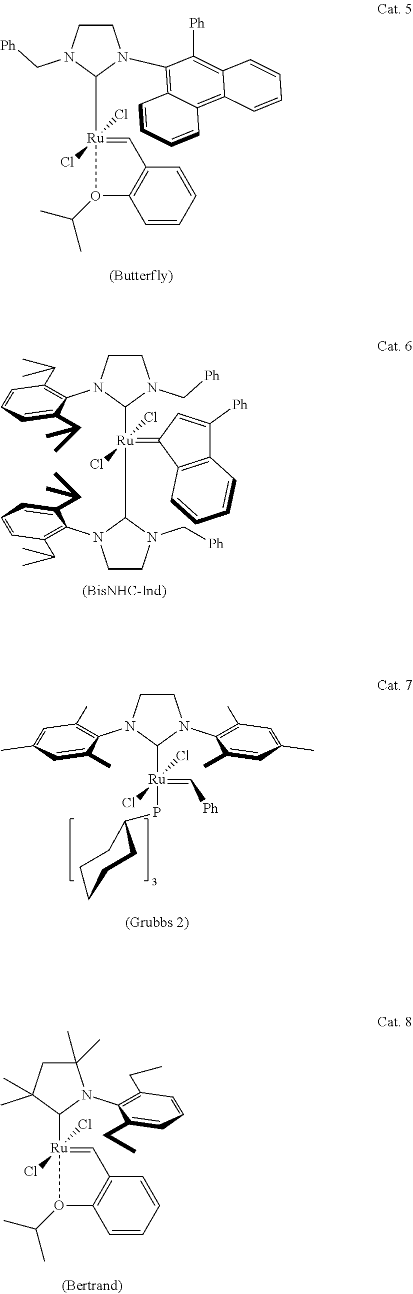

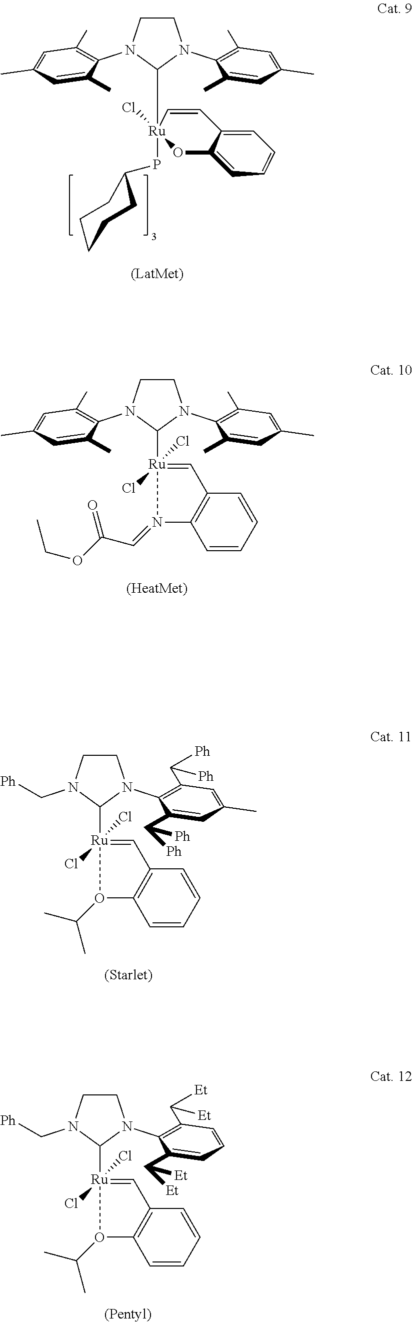

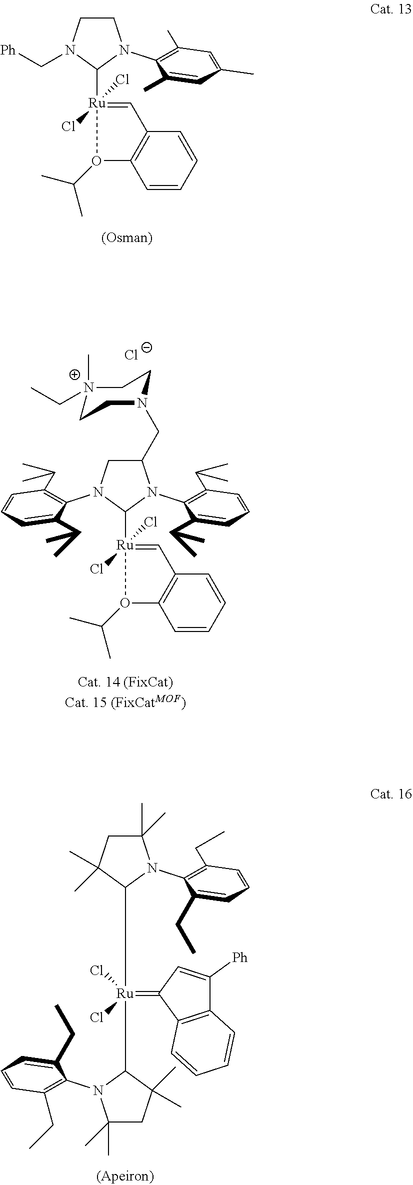

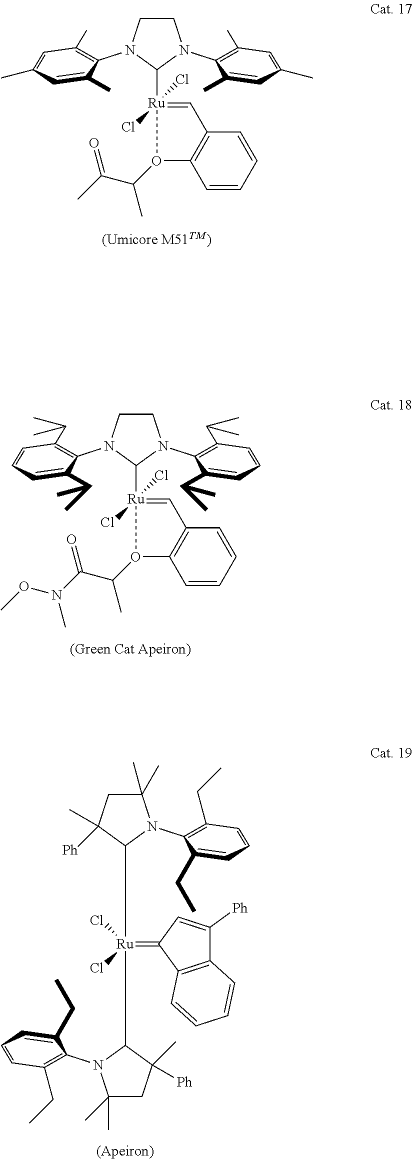

Preferably, compound 1 is deposited on a solid support selected from the group comprising silica gel (SiO₂), aluminium oxide (Al₂O₃), zeolites, celite, or MOF (Metal Organic Framework)-like materials, i.e. potentially porous coordination polymers.

Preferably, in said reaction, quinone derivatives are used as an additive in an amount from 5 mol % to 0.05 mol %, preferably such as quinone, anthraquinone, tetrafluoroquinone, tetrachloroquinone and the like.

Preferably, the reaction is conducted in an organic solvent such as toluene, benzene, mesitylene, dichloromethane, ethyl acetate, methyl acetate, tert-butyl methyl ether, cyclopentyl methyl ether, paraffin oil, paraffin wax, ionic liquid, polyethylene, PAO polyalphaolefins, preferably PAO 6 and PAO 4, or without any solvent.

Preferably, olefins Dx and/or olefin Dy at a concentration of between 1 mM and 1 M are used in the reaction.

Preferably, the reaction is conducted at a temperature of between 20 and 200° C. for between 5 minutes and 24 hours.

Preferably, compound 1 is used in an amount between 2 mol % and 0.0005 mol %.

Preferably, compound 2 is added to the reaction mixture in portions and/or continuously using a pump, as a solid and/or as a solution in an organic solvent.

Preferably, olefin Dx or olefin Dy is added to the reaction mixture in portions and/or continuously using a pump.

Preferably, during the reaction, the reaction product that is gaseous in the reaction conditions is actively removed from the reaction mixture using inert gas or vacuum.

Preferably, the reaction is conducted at a pressure below the atmospheric pressure, more preferably at a pressure of between 1 bar and 1·10⁻⁶ mbar.

The invention also relates to a method for producing a cyclic compound Mx that contains at least one non-terminal C═C double bond,

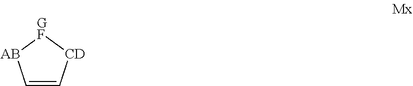

wherein:

AB and CD are each independently a group selected from such as C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

characterised in that the olefin Dx with the following formula

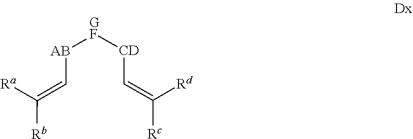

wherein:

AB and CD are each independently a group selected from such as a hydrogen atom, C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be optionally substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

R^{a}, R^{b}, R^{c} i R^{d} are each independently a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

or the olefin Dy with the formula

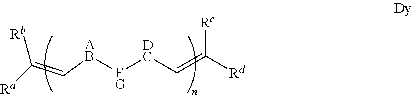

wherein:

AB and CD are each independently a group selected from such as a hydrogen atom, C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be optionally substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

R^{a}, R^{b}, R^{c} i R^{d} are each independently a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;

are subjected to olefin metathesis reaction with the compound of the formula 1,

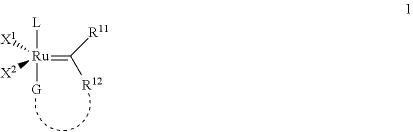

wherein:

X¹ and X² are each independently an anionic ligand selected from such as halogen atoms, —CN, —SCN, —OR′, —SR′, —O(C═O)R′, —O(SO₂)R′, —OSi(R′)₃ group, wherein R′ is C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy, or a halogen atom;

R¹¹, R¹² are each independently a hydrogen atom, a halogen atom, optionally substituted C₁-C₂₅ alkyl, optionally substituted C₁-C₂₅ perfluoralkyl, optionally substituted C₂-C₂₅ alkene, optionally substituted C₃-C₇ cycloalkyl, optionally substituted C₂-C₂₅ alkenyl, optionally substituted C₃-C₂₅ cycloalkenyl, optionally substituted C₂-C₂₅ alkinyl, optionally substituted C₃-C₂₅ cycloalkinyl, optionally substituted C₁-C₂₅ alkoxy, optionally substituted C₅-C₂₄ aryloxy, optionally substituted C₅-C₂₀ heteroaryloxy, optionally substituted C₅-C₂₄ aryl, optionally substituted C₅-C₂₀ heteroaryl, optionally substituted C₇-C₂₄ aralkyl, optionally substituted C₅-C₂₄ perfluoroaryl, optionally substituted 3-12-membered heterocycle;

wherein the substituents R¹¹ and R¹² may be interconnected to form a ring selected from the group consisting of C₃-C₇ cycloalkyl, C₃-C₂₅ cycloalkenyl, C₃-C₂₅ cycloalkinyl, C₅-C₂₄ aryl, C₅-C₂₀ heteroaryl, C₅-C₂₄ perfluoroaryl, 3-12-membered heterocycle, each of which may be independently substituted with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle, in which case the dotted line between G and R¹² is not a chemical bond;

wherein the substituents R¹¹ i R¹² preferably are a hydrogen atom or aryl independently substituted with the following groups: alkoxy (—OR′), sulfide (—SR′), sulfoxide (—S(O)R′), sulfonium (—S⁺R′₂), sulphonic (—SO₂R′), sulfonamide (—SO₂NR′₂), amino (—NR′₂), ammonium (—NR′₃), nitro (—NO₂), cyano (—CN), phosphonium (—P(O)(OR′)₂), phosphinium (—P(O)R′(OR′)), phosphonous (—P(OR′)₂), phosphine (—PR′₂), phosphine oxides (—P(O)R′₂), phosphonium (—P⁺R′₃), carboxy (—COOH), ester (—COOR′), amide (—CONR′₂), amide (—NR′COR″), formyl (—CHO), ketone (—COR′), in which groups R′ is C₁-C₅ alkyl, C₁-C₅ perfluoroalkyl, C₅-C₂₄ aryl, C₇-C₂₄ aralkyl, C₅-C₂₄ perfluoroaryl;

L is selected from such as:

a)

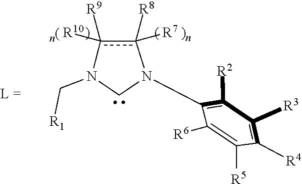

wherein:

R¹ is a heteroaryl group;

R², R³, R⁴, R⁵, R⁶ are each independently a hydrogen atom, a C₁-C₂₅ alkyl group, a C₁-C₂₅ alkoxy group or a C₂-C₂₅ alkenyl group, wherein the substituents R², R³, R⁴, R⁵, R⁶ may be interconnected to form a substituted or unsubstituted cyclic C₄-C₁₀ or polycyclic C₄-C₁₂ system;

R⁷, R⁸, R⁹, i R¹⁰ are each independently a hydrogen atom or a C₁-C₂₅ alkyl group, or R⁷ and/or R⁸ can be connected with R⁹ and/or R¹⁰ to form a cyclic system;

n is 0 or 1;

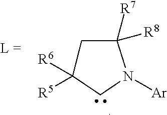

wherein:

Ar is an aryl group which is substituted with hydrogen atoms or is optionally substituted with at least one of the following groups: C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, C₇-C₂₄ aralkyl, C₅-C₂₄ perfluoroaryl, or a halogen atom;

R⁵, R⁶, R⁷ and R⁸ are each independently a hydrogen atom or one of the following groups: C₁-C₂₅ alkyl, C₃-C₁₂ cycloalkyl, C₁-C₅ perfluoroalkyl, C₂-C₁₂ alkenyl, C₅-C₂₀ aryl, C₅-C₂₄ aryloxy, C₂-C₂₀ heterocyclo, C₄-C₂₀ heteroarylo, C₅-C₂₀ heteroaryloxy, C₇-C₂₄ aralkyl, C₅-C₂₄ perfluoroaryl, which are optionally substituted with at least one C₁-C₁₂ alkyl, C₁-C₁₂ perfluoroalkyl, C₁-C₁₂ alkoxy, C₅-C₂₄ aryloxy, C₄-C₂₀ heteroaryl, C₅-C₂₀ heteroaryloxy, or a halogen atom; moreover, R⁵ and R⁶ and/or R⁷ i R⁸ may be interconnected to form a cyclic system;

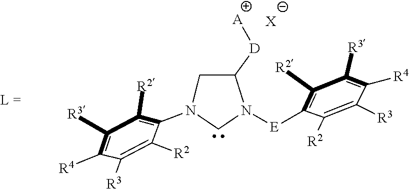

wherein:

the combination A⁺X⁻ and D is a hydrogen atom, or

A is independently a substituent containing a tertiary amine group or a quaternary ammonium group, which may be a N(R¹)(R²) or N(R¹)(R²)(R³) group, wherein R¹, R² and R³ are each independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl; alternatively, A is one of the following groups: C₁-C₂₅ cycloaminoalkyl, C₁-C₂₅ cyclodiaminoalkyl, C₁-C₂₅ cyclotriaminoalkyl, C₁-C₂₅ cyclotetraaminoalkyl, C₁-C₂₅ cycloaminoammonioalkyl, wherein the at least one nitrogen atom is independently substituted with at least one R¹ group, wherein R¹ is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl so that at least one nitrogen atom in the ring forms a quaternary ammonium group;

X is independently a halogen atom, or CF₃SO₃⁻, BF₄⁻, PF₆⁻, and ClO₄⁻;

D is independently one of the following groups: C₁-C₂₅, alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl, or C₂-C₂₅ alkenyl, C₁-C₂₅ α,ω-dialkoxy, (CH₂CH₂O)ₙ, polyether, where n comprises from 1 to 25, a C₁-C₂₅ thioalkyl group, a C₁-C₂₅ α,ω-dithioalkyl group, a C₁-C₂₅ α,ω-diheteroalkyl group, a C₁-C₂₅ aminoalkyl group;

E is a single bond or independently a C₁-C₂₅ alkyl group, a C₁-C₁₂ perfluoroalkyl group, a C₃-C₇ cycloalkyl group, a C₁-C₂₅ alkoxy group;

X¹ and X² are each independently an anion ligand selected from such as halogen atoms;

R², R^{2′}, R³, R^{3′} i R⁴ are each independently a hydrogen atom, a halogen atom, a C₁-C₂₅ alkyl group, a C₃-C₇ cycloalkyl group, a C₁-C₂₅ alkoxy group, a C₅-C₂₄ perfluoroaryl group, a C₅-C₂₀ heteroaryl group or a C₂-C₂₅ alkenyl group, wherein the substituents R², R^{2′}, R³, R^{3′} and R⁴ may be interconnected to form a substituted or unsubstituted cyclic C₄-C₁₀ or polycyclic C₄-C₁₂ system;

and

G is selected from the substituents L listed above or G is a heteroatom selected from the group comprising an oxygen, nitrogen, sulphur, phosphorus, fluorine, chlorine, bromine and iodine atom, optionally substituted with a group selected from such as hydrogen atom, C₁-C₂₅ alkyl, C₁-C₂₅ perfluoroalkyl, C₃-C₇ cycloalkyl, C₅-C₂₄ aryl, C₅-C₂₄ perfluoroaryl, C₅-C₂₀ heteroaryl, C₇-C₂₄ aralkyl, 3-12 membered heterocycle, from the following groups: —COR′ acyl, (—CN) cyano, (—COOH) carboxy, (—COOR′) ester, (—CONR′₂) amide, (—SO₂R′) sulfonic, (—CHO) formyl, (—SO₂NR′₂) sulfonamide, (—COR′) ketone, wherein the R′ group is C₁-C₅ alkyl, C₁-C₅ perfluoroalkyl, C₅-C₂₄ aryl, C₅-C₂₄ perfluoroaryl, C₇-C₂₄ aralkyl, in which case the dotted line is a direct bond between the heteroatom and the R¹² substituent, in the form of an aryl optionally substituted by 1-4 substituents independently selected from the group comprising a hydrogen atom, a halogen atom, C₁-C₂₅ alkyl, C₁-C₂₅ perfluoroalkyl, C₂-C₂₅ alken, C₃-C₇ cycloalkyl, C₂-C₂₅ alkenyl, C₃-C₂₅ cycloalkenyl, C₂-C₂₅ alkinyl, C₃-C₂₅ cycloalkinyl, C₅-C₂₄ aryl, C₇-C₂₄ aralkyl, C₅-C₂₄ perfluoroaryl, C₅-C₂₀ heteroaryl, 3-12-membered heterocycle, one of the following groups: (—OR′) alkoxy, (—SR′) sulfide, (—NO₂) nitro, (—CN) cyano, (—COOH) carboxy, (—COOR′) ester, (—CONR′₂) amide, (—CONR′COR′) imide, (—NR′₂) amino, (—NR′₃) ammonium, (—NR′COR′) amide, (—NR′SO₂R′), sulfonamide (—SO₂R′), sulfonic (—CHO), formyl (—SO₂NR′₂), sulfonamide (—COR′), ketone, in which groups R′ is as follows: C₁-C₅ alkyl, C₁-C₅ perfluoroalkyl, C₅-C₂₄ aryl, C₅-C₂₄ perfluoroaryl, C₇-C₂₄ aralkyl;

wherein the metathesis reaction is optionally carried out in the presence of other additives enhancing the process of the reaction.

Preferably, compound 1a is used as compound 1.

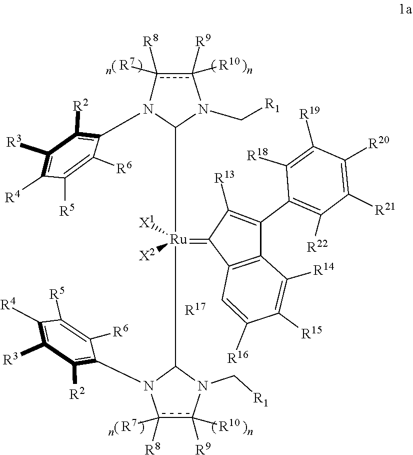

wherein:

X¹, X² are a halogen atom;

R¹ is a heteroaryl selected from the group comprising furan, thiophene, benzothiophene, benzofuran;

R², R³, R⁴, R⁵, R⁶ are each independently a hydrogen atom, methyl, isopropyl, a halogen atom;

R⁷, R⁸, R⁹, R¹⁰ are each independently a hydrogen atom or methyl;

n is 0 or 1;

R¹³, R¹⁴, R¹⁵, R¹⁶, R¹⁷, R¹⁸, R¹⁹, R²⁰, R²¹ and R²² are each independently a hydrogen atom, a halogen atom, one of the following groups: C₁-C₂₅ alkyl, C₁-C₂₅ alkylamino, C₁-C₂₅ alkylammonium, C₁-C₂₅ perfluoroalkyl, C₂-C₂₅ alkenyl, C₃-C₇ cycloalkyl, C₃-C₂₅ cycloalkenyl, C₂-C₂₅ alkynyl, C₃-C₂₅ cycloalkynyl, C₁-C₂₅ alkoxy, C₅-C₂₄ aryl, C₅-C₂₀ heteroaryl, C₃-C₁₂ heterocycle, 3-12-membered heterocycle, a sulfide (—SR′), ester (—COOR′), amido (—CONR′₂), sulfonic (—SO₂R′), sulfonamide (—SO₂NR′₂) or ketone (—COR′), group, wherein R′ is a C₁-C₅ alkyl, C₁-C₅ perfluoroalkyl, C₅-C₂₅ aryl or C₅-C₂₅ perfluoroaryl group.

Preferably, compound 1b is used as compound 1

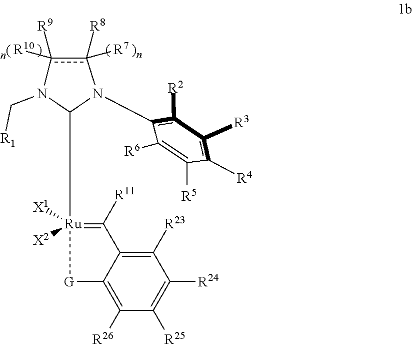

wherein:

X¹, X² are a halogen atom;

R¹ is a heteroaryl selected from the group comprising furan, thiophene, benzothiophene, benzofuran;

R², R³, R⁴, R⁵, R⁶ are each independently a hydrogen atom, methyl, isopropyl, a halogen atom;

R⁷, R⁸, R⁹, R¹⁰ are each independently a hydrogen atom or methyl;

n is 0 or 1;

R¹¹ is a hydrogen atom;

R²³, R²⁴, R²⁵, R²⁶ are each independently a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₅-C₂₄ aryl, C₇-C₂₄ aralkyl, C₅-C₂₄ perfluoroaryl, C₅-C₂₀ heteroaryl, 3-12 membered heterocycle, one of the following groups: alkoxy (—OR′), sulfide (—SR′), nitro (—NO₂), cyano (—CN), carboxy (—COOH), ester (—COOR′), amido (—CONR′₂), imido (—CONR′COR′), amino (—NR′₂), ammonium (—N⁺R′₃), amide (—NR′COR′), sulfonamide (—NR′SO₂R′), sulfonate (—SO₂R′), formyl (—CHO), sulfonamide (—SO₂NR′₂), ketone (—COR′), in which R′ groups are as follows: C₁-C₅ alkyl, C₁-C₅ perfluoroalkyl, C₅-C₂₄ aryl, C₅-C₂₄ perfluoroaryl, C₇-C₂₄ aralkyl, wherein R²³, R²⁴, R²⁵, R²⁶ are preferably a hydrogen atom;

G is a halogen atom or a substituent selected from the group OR′, SR′, S(O)R′, S(O)₂R′ N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom.

Preferably, compound 1c is used as compound 1

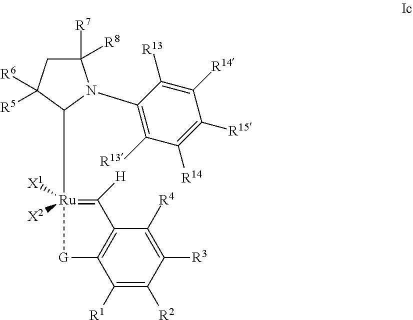

wherein:

X¹, X² are a halogen atom;

G is a halogen atom or a substituent selected from the group OR′, SR′, S(O)R′, S(O)₂R′ N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;

R¹, R², R³, R⁴ are each independently a hydrogen atom, a sulfoxide group (—S(O)R′), a sulfonamide group (—SO₂NR′₂), a nitro group (—NO₂), an ester group (—COOR′), a ketone group (—COR′), a —NC(O)R′ammonium group, a (—OMe) alkoxy group, in which groups R′ is C₁-C₅ alkyl, C₁-C₅ perfluoroalkyl, C₅-C₂₄ aryl, C₇-C₂₄ aralkyl, C₅-C₂₄ perfluoroaryl;

R⁵, R⁶, R⁷, i R⁸ are each independently a hydrogen atom or a C₁-C₂₅ alkyl group, a C₅-C₂₀ aryl group, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom; moreover, R⁵ and R⁶ and/or R⁷ and R⁸ may be interconnected to form a cyclic system

R¹³ and R^{13′} are each independently methyl or ethyl;

R¹⁴, R^{14′}, R¹⁵ are each independently a hydrogen atom, a C₁-C₂₅ alkyl group.

Preferably, compound 1d is used as compound 1

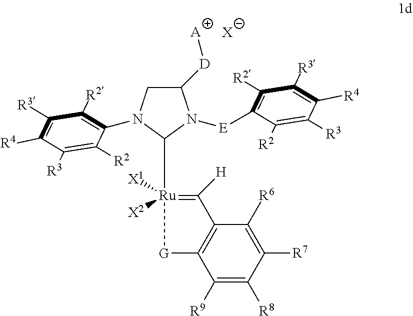

wherein:

the combination A⁺X⁻ and D is a hydrogen atom, or

A is independently a substituent containing a tertiary amine group or a quaternary ammonium group, which may be a N(R¹)(R²) or N(R¹)(R²)(R³) group, wherein R¹, R² and R³ are each independently one of the following groups: C₁-C₂₅ alkyl C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl; alternatively, A is one of the following groups: C₁-C₂₅ cycloaminoalkyl, C₁-C₂₅ cyclodiaminoalkyl, C₁-C₂₅ cyclotriaminoalkyl, C₁-C₂₅ cyclotetraaminoalkyl, C₁-C₂₅ cycloaminoammonioalkyl, wherein the at least one nitrogen atom is independently substituted with at least one R¹ group, wherein R¹ is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl so that at least one nitrogen atom in the ring forms a quaternary ammonium group;

X is independently a halogen atom, or CF₃SO₃⁻, BF₄⁻, PF₆⁻, and ClO₄⁻;

D is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl, or C₂-C₂₅ alkenyl, C₁-C₂₅ α,ω-dialkoxy group, a (CH₂CH₂O)ₙ polyether group, where n comprises from 1 to 25, a C₁-C₂₅ thioalkyl group, a C₁-C₂₅ α,ω-dithioalkyl group, a C₁-C₂₅ α,ω-diheteroalkyl group, a C₁-C₂₅ aminoalkyl group;

E is independently a C₁-C₂₅ alkyl group, a C₁-C₁₂ perfluoroalkyl group, a C₃-C₇ cycloalkyl group, a C₁-C₂₅ alkoxy group, or a single bond;

X¹ and X² are each independently an anion ligand selected from such as halogen atoms;

R¹ is independently a C₁-C₂₅ alkyl group, a C₃-C₇ cycloalkyl group, a C₅-C₂₄ aryl group, a C₅-C₂₀ heteroaryl group;

R², R^{2′}, R³, R^{3′} i R⁴ are each independently a hydrogen atom, a halogen atom, a C₁-C₂₅ alkyl group, a C₃-C₇ cycloalkyl group, a C₁-C₂₅ alkoxy group, a C₅-C₂₄ perfluoroaryl group, a C₅-C₂₀ heteroaryl group or a C₂-C₂₅ alkenyl group, wherein the substituents R², R^{2′}, R³, R^{3′} and R⁴ may be interconnected to form a substituted or unsubstituted cyclic C₄-C₁₀ or polycyclic C₄-C₁₂ system;

R⁵ is a hydrogen atom, a C₁-C₂₅ alkyl group, a C₃-C₇ cycloalkyl group;

R⁶, R⁷, R⁸, R⁹ are each independently a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₅-C₂₄ aryl, C₇-C₂₄ aralkyl, C₅-C₂₄ perfluoroaryl, C₅-C₂₀ heteroaryl, 3-12 membered heterocycle, one of the following groups: alkoxy (—OR′), sulfide (—SR′), nitro (—NO₂), cyano (—CN), carboxy (—COOH), ester (—COOR′), amido (—CONR′₂), imido (—CONR′COR′), amino (—NR′₂), ammonium (—N⁺R′₃), amide (—NR′COR′), sulfonamide (—NR′SO₂R′), sulfonate (—SO₂R′), formyl (—CHO), sulfonamide (—SO₂NR′₂), ketone (—COR′), in which R′ groups are as follows: C₁-C₅ alkyl, C₁-C₅ perfluoroalkyl, C₅-C₂₄ aryl, C₅-C₂₄perfluoroaryl, C₇-C₂₄ aralkyl, wherein R²³, R²⁴, R²⁵, R²⁶ are preferably a hydrogen atom;

G is a halogen atom or a substituent selected from the group OR′, SR′, S(O)R′, S(O)₂R′ N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;

Preferably, compound If is used as compound 1

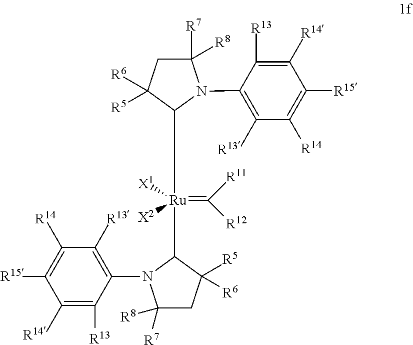

wherein:

X¹ and X² are each independently an anionic ligand selected from such as halogen atoms, —CN, —SCN, —OR′, —SR′, —O(C═O)R′, —O(SO₂)R′, —OSi(R′)₃ group, wherein R′ is C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy, or a halogen atom;

R⁵, R⁶, R⁷ and R⁸ are each independently a hydrogen atom or a C₁-C₂₅ alkyl group, R⁷ and/or R⁸ may be interconnected with R⁹ and/or R¹⁰ to form a cyclic system, they also may be independently the following groups: C₁-C₁₂ alkyl, C₃-C₁₂ cycloalkyl, C₂-C₁₂ alkenyl C₅-C₂₀ aryl, C₁-C₅ perfluoralkyl, C₇-C₂₄ aralkyl, C₅-C₂₄ perfluoroaryl, which are optionally substituted with at least one C₁-C₁₂ alkyl, C₁-C₁₂perfluoroalkyl, C₁-C₁₂ alkoxy, C₅-C₂₄ aryloxy, C₅-C₂₀ heteroaryloxy or a halogen atom;

R¹¹, R¹² are each independently a hydrogen atom, a halogen atom, optionally substituted C₁-C₂₅ alkyl, optionally substituted C₁-C₂₅ perfluoralkyl, optionally substituted C₂-C₂₅ alkene, optionally substituted C₃-C₇ cycloalkyl, optionally substituted C₂-C₂₅ alkenyl, optionally substituted C₃-C₂₅ cycloalkenyl, optionally substituted C₂-C₂₅ alkinyl, optionally substituted C₃-C₂₅ cycloalkinyl, optionally substituted C₁-C₂₅ alkoxy, optionally substituted C₅-C₂₄ aryloxy, optionally substituted C₅-C₂₀ heteroaryloxy, optionally substituted C₅-C₂₄ aryl, optionally substituted C₅-C₂₀ heteroaryl, optionally substituted C₇-C₂₄ aralkyl, optionally substituted C₅-C₂₄ perfluoroaryl, optionally substituted 3-12-membered heterocycle;

wherein the substituents R¹¹ and R¹² may be interconnected to form a ring selected from the group consisting of C₃-C₇ cycloalkyl, C₃-C₂₅ cycloalkenyl, C₃-C₂₅ cycloalkinyl, C₅-C₂₄ aryl, C₅-C₂₀ heteroaryl, C₅-C₂₄ perfluoroaryl, 3-12-membered heterocycle, each of which may be substituted with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;

R¹³ and R^{13′} are each independently methyl or ethyl;

R¹⁴, R^{14′}, R¹⁵ are each independently a hydrogen atom, a C₁-C₂₅ alkyl group.

Preferably, compound selected from such as those below is used as compound 1.

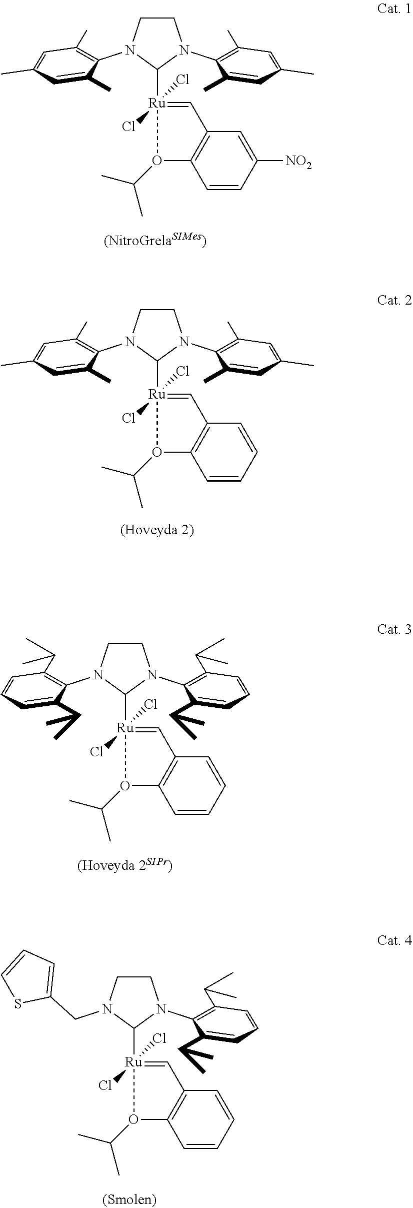

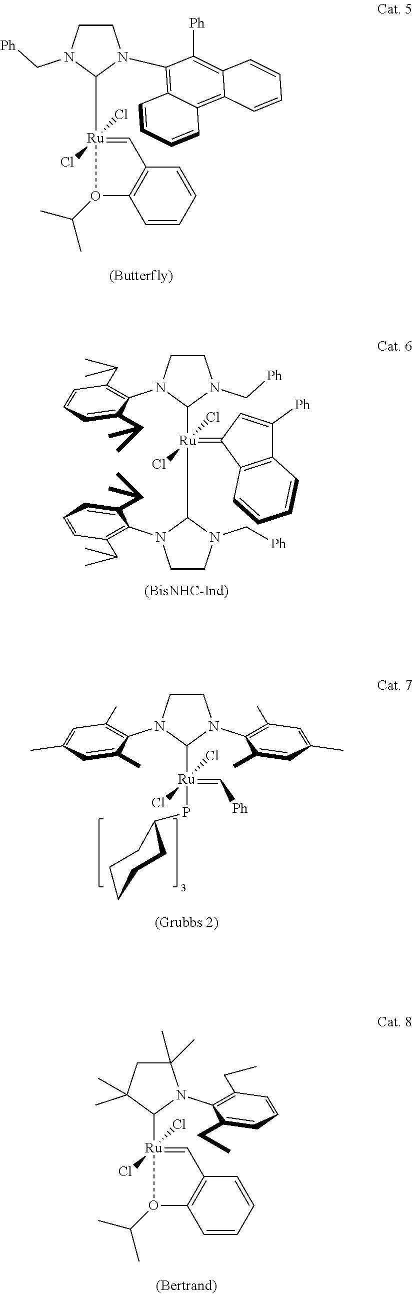

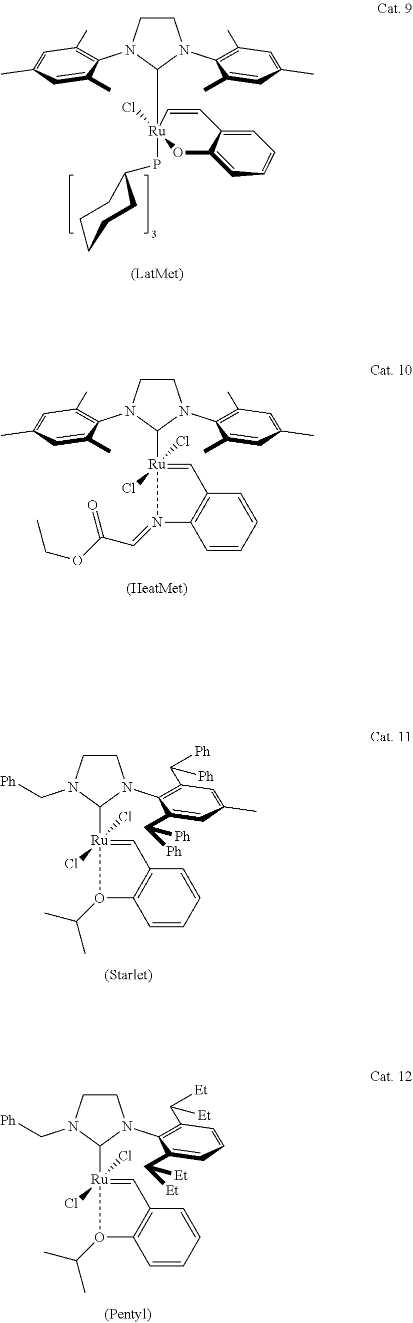

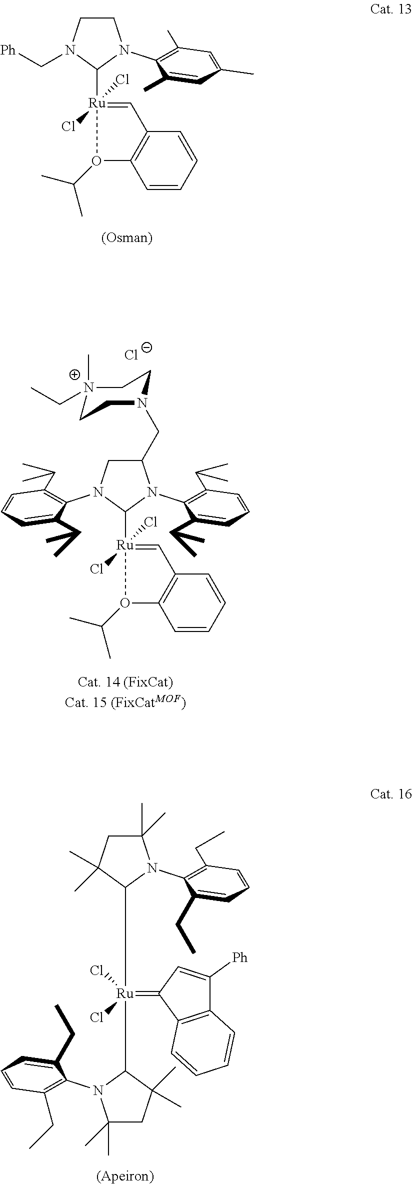

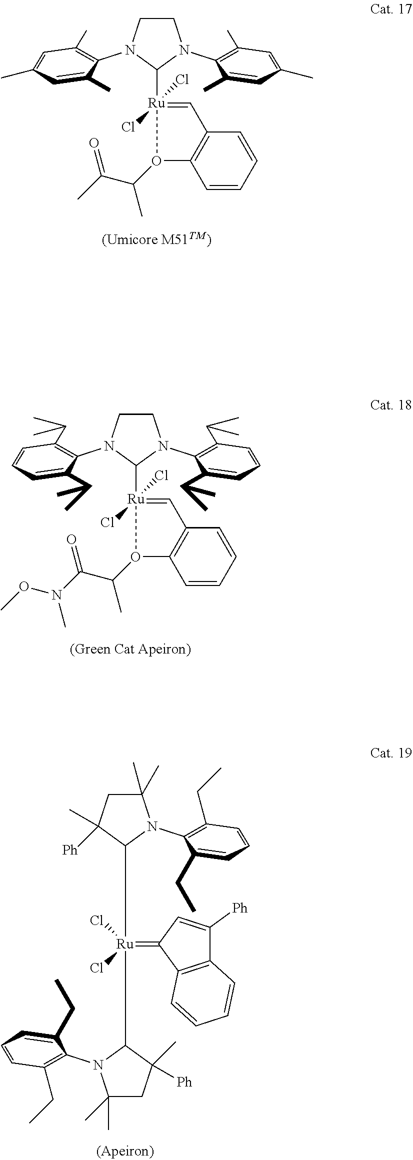

Preferably, compound 1 is used deposited on a solid support selected from the group comprising silica gel (SiO₂), aluminium oxide (Al₂O₃), zeolites, celite, or MOF (Metal Organic Framework)-like materials, i.e. potentially porous coordination polymers.

Preferably, quinone derivatives are used as an additive in an amount from 5 mol % to 0.05 mol %, preferably such as quinone, anthraquinone, tetrafluoroquinone, tetrachloroquinone and the like.

Preferably, the reaction is conducted in an organic solvent such as toluene, benzene, mesitylene, dichloromethane, ethyl acetate, methyl acetate, tert-butyl methyl ether, cyclopentyl methyl ether, paraffin oil, paraffin wax, ionic liquid, polyethylene, PAO polyalphaolefins, preferably PAO 6 and PAO 4, or without any solvent.

Preferably, the reaction is conducted with olefin Dx and/or olefin Dy at a concentration of between 1 mM and 1 M.

Preferably, the reaction is conducted at a temperature of between 20 and 200° C. for between 5 minutes and 24 hours.

Preferably, compound 1 is used in an amount between 2 mol % and 0.0005 mol %.

Preferably, compound 1 is added to the reaction mixture in portions and/or continuously using a pump.

Preferably, compound 1 is added to the reaction mixture as a solid and/or as a solution in an organic solvent.

Preferably, olefin Dx and/or olefin Dy is added to the reaction mixture in portions and/or continuously using a pump.

Preferably, the reaction product that is gaseous in the reaction conditions is actively removed from the reaction mixture using inert gas or vacuum.

Preferably, the reaction is conducted at a pressure below the atmospheric pressure, more preferably at a pressure of between 1 bar and 1·10⁻⁶ mbar.

The invention will be presented in greater detail in preferred embodiments, with reference to the accompanying drawing, in which:

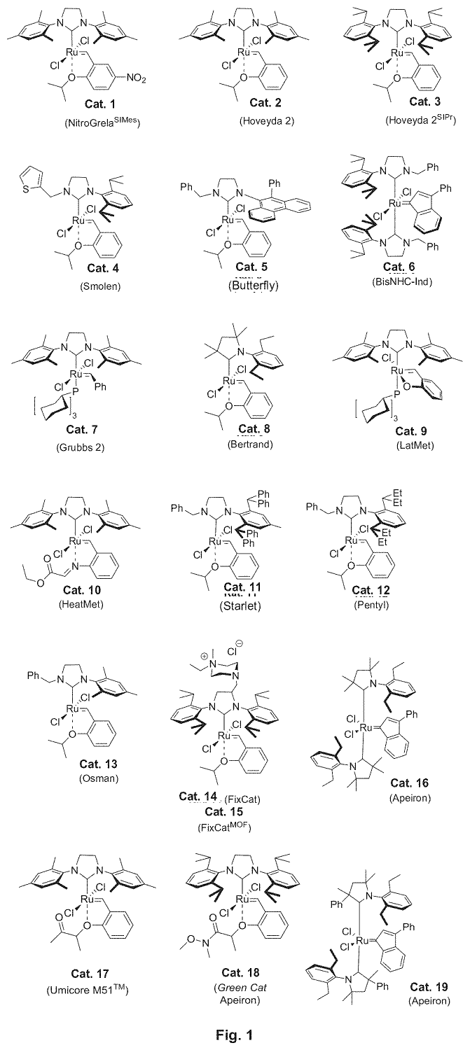

FIG. 1 shows ruthenium complexes used as catalysts and/or precatalysts with respective assays (FixCat^{MOF} is a heterogeneous catalyst)

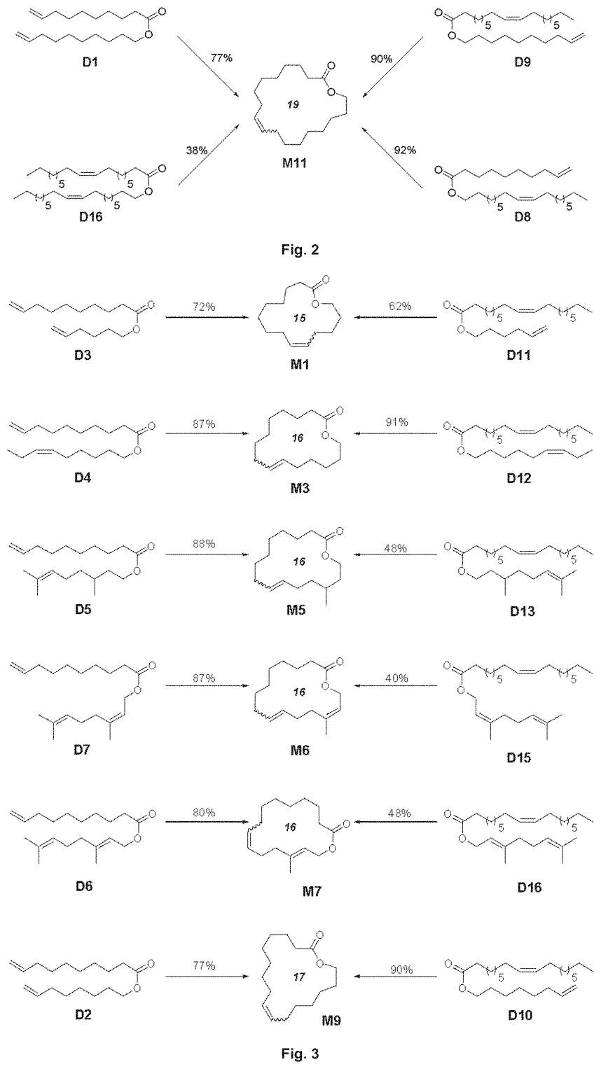

FIG. 2 shows different dienes (Dx), which, upon being subjected to the RCM reaction, yield the M11 macrocycle.

FIG. 3 shows macrocyclisations of dienes (Dx) yielding 15-, 16- and 17-membered macrocycles (Mx).

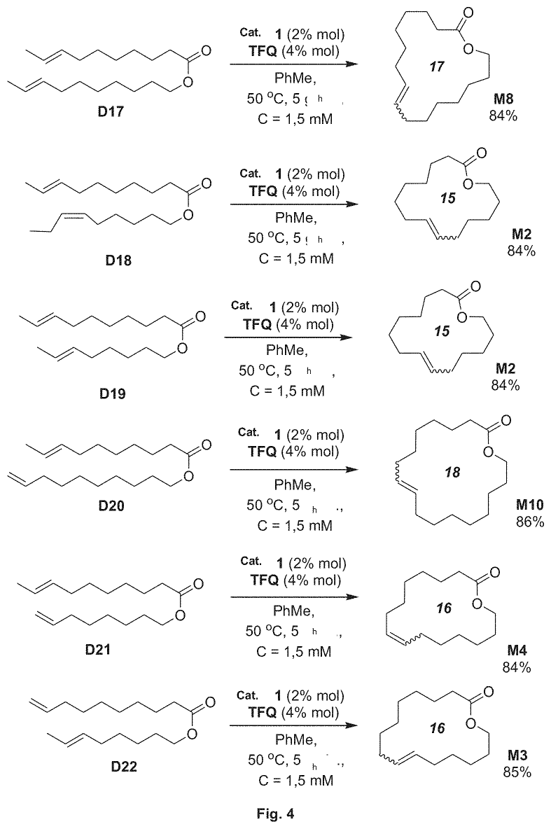

FIG. 4 shows macrocyclisations of dienes (Dx) comprising one (or both) C═C double bonds substituted with methyl (—CH₃) or ethyl (—CH₂CH₃).

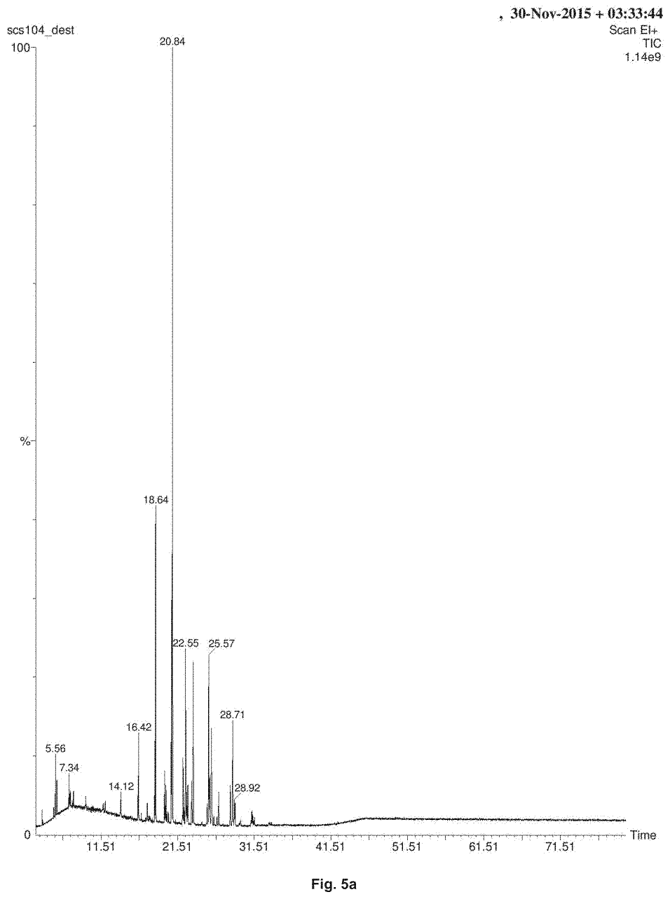

FIG. 5a shows the gas chromatogram of macrocyclisation conducted by way of the RCM reaction under non-optimised conditions, wherein the large number of signals in the chromatogram indicates non-selectivity of the process (D10=>M9). Reaction conditions: oct-7-en-1-yl oleate (D10, 0.5 g; 1.28 mmol); catalyst Cat. 5 (4 mol %); quinone (8 mol %); paraffin oil as solvent (2 g); temperature=150° C.; reaction time=4 hours; pressure of the order of 1·10⁻³ mbar.

FIG. 5b shows the gas chromatogram of macrocyclisation conducted by way of the RCM reaction under optimised conditions (D12=>M3). Reaction conditions: cis-non-6-en-1-yl oleate (D12, 0.203 g); catalyst Cat. 5 (0.5% mol); paraffin oil as solvent (2 g); temperature=110° C.; reaction time=8 hours; pressure of the order of 1.10⁻⁶ mbar (during the reaction, the pressure varied between approx. 7.10⁻⁶ mbar and 2.10⁻⁶ mbar)

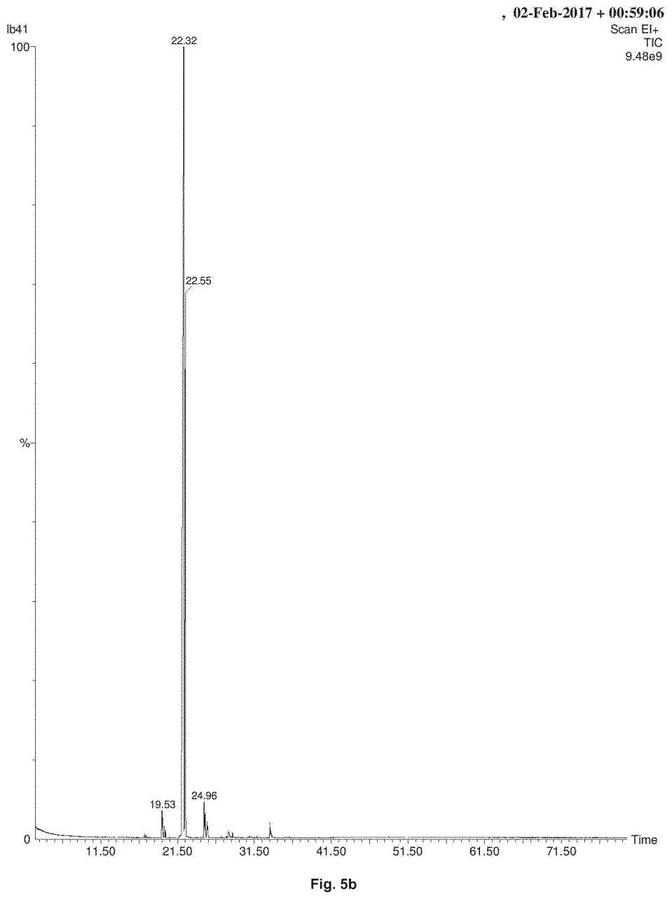

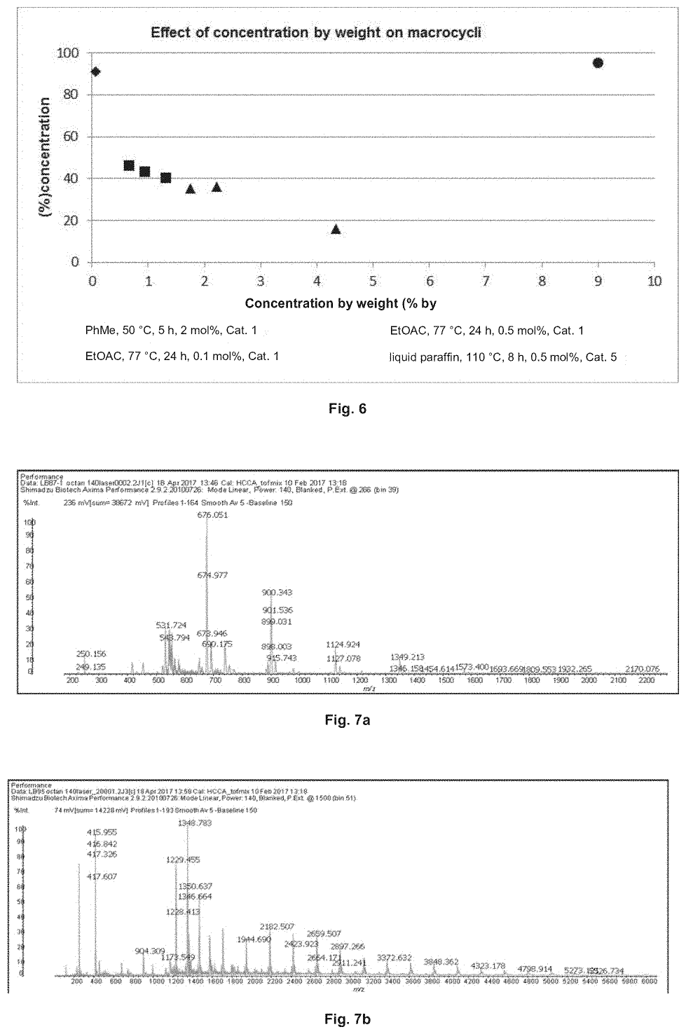

FIG. 6 shows a graph of the dependence of the RCM reaction yield and concentration by weight (depending on various conditions).

FIGS. 7a and 7b. Results of the analysis using the MALDI-TOF MS spectrometry method of example XV.

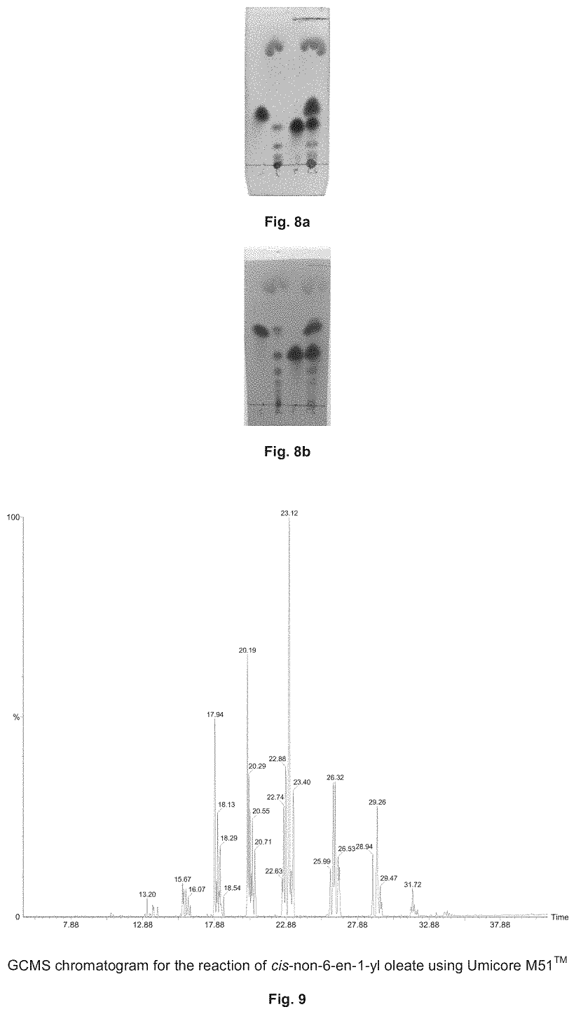

FIGS. 8a and 8b. Result of the analysis using the thin-layer chromatography of example XV.

FIG. 9 GCMS chromatogram for the reaction of cis-non-6-en-1-yl oleate using Umicore M51™

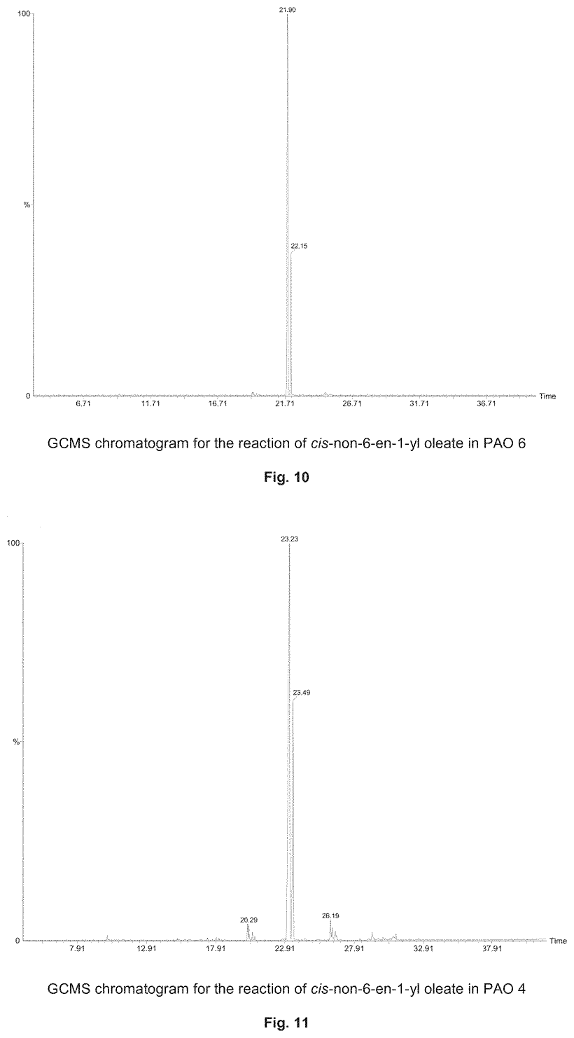

FIG. 10 GCMS chromatogram for the reaction of cis-non-6-en-1-yl oleate in PAO 6

FIG. 11 GCMS chromatogram for the reaction of cis-non-6-en-1-yl oleate in PAO 4

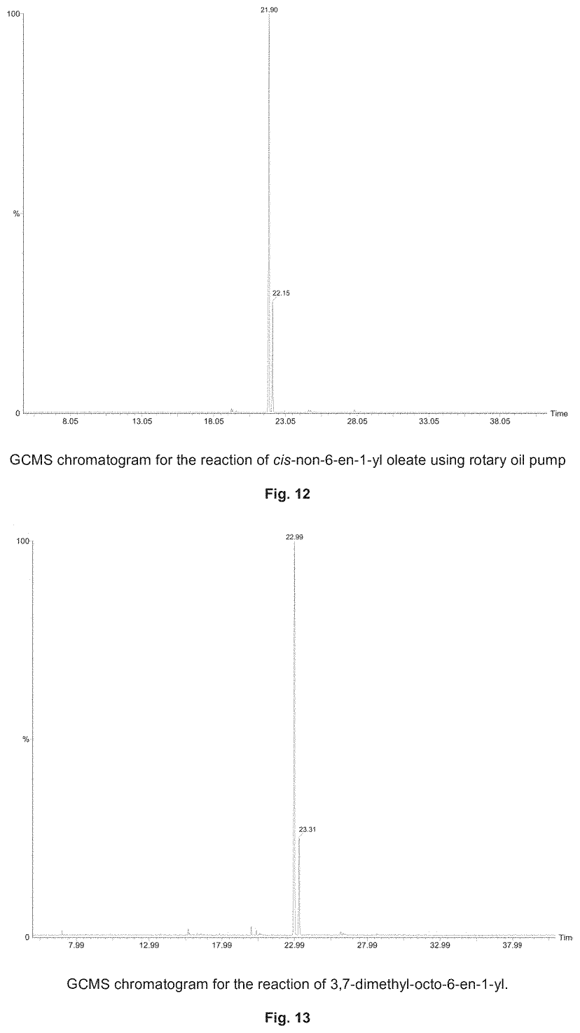

FIG. 12 GCMS chromatogram for the reaction of cis-non-6-en-1-yl oleate using rotary oil pump.

FIG. 13 GCMS chromatogram for the reaction of 3,7-dimethyl-oct-6-en-1-yl.

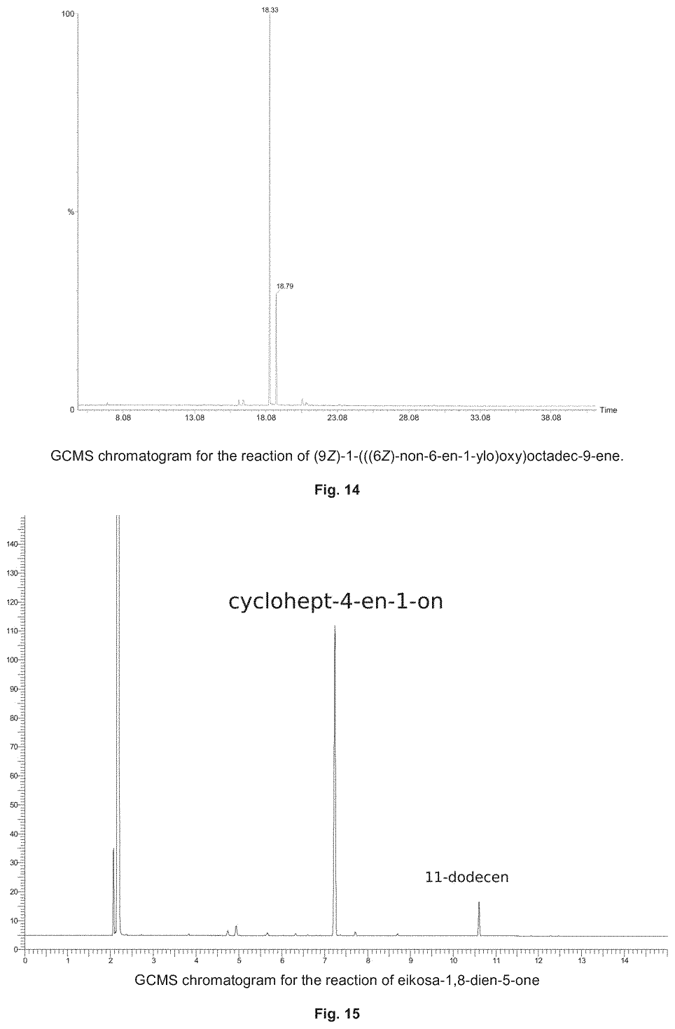

FIG. 14 GCMS chromatogram for the reaction of (9Z)-1-(((6Z)-non-6-en-1-ylo)oxy)octadec-9-ene.

FIG. 15 GCMS chromatogram for the reaction of eikosa-1,8-dien-5-on.

### TERMS

The terms used in the present description have the meanings as follows. Non-defined terms have the meaning understood by a person skilled in the art in the light of the best knowledge held, of the present disclosure, and of the context of the description of the patent application. Unless it is indicated otherwise, the following conventional chemistry terms are used the present description that have the meanings indicated in the definitions below.

The term “halogen atom” or “halogen” refers to an element selected from F, Cl, Br, I.

The term “carbene” refers to a particle containing an neutral carbon atom with a valence number of two and having two unpaired (triplet state) or paired (singlet state) valence electrons. The term “carbene” also includes carbene analogs in which the carbon atom is substituted by another chemical element such as boron, silicon, germanium, tin, lead, nitrogen, phosphorus, sulphur, selenium and tellurium.

The term “alkyl” refers to a saturated, linear or branched hydrocarbon substituent having the indicated number of carbon atoms. Examples of alkyl substituents include -methyl, -ethyl, -n-propyl, -n-butyl, -n-pentyl, -n-hexyl, -n-heptyl, -n-octyl, -n-nonyl, and -n-decyl. Representative branched —(C₁-C₁₀)alkyls include -isopropyl, -sec-butyl, -isobutyl, -tert-butyl, -isopentyl, -neopentyl, -1-methylobutyl, -2-methylobutyl, -3-methylobutyl, -1,1-dimethylopropyl, -1,2-dimethylopropyl, -1-methylopentyl, -2-methylopentyl, -3-methylopentyl, -4-methylopentyl, -1-ethylobutyl, -2-ethylobutyl, -3-ethylobutyl, -1,1-dimethylobutyl, -1,2-dimethylobutyl, 1,3-dimethylobutyl, -2,2-dimethylobutyl, -2,3-dimethylobutyl, -3,3-dimethylobutyl, -1-methylohexyl, 2-methylohexyl, -3-methylohexyl, -4-methylohexyl, -5-methylohexyl, -1,2-dimethylopentyl, -1,3-dimethylopentyl, -1,2-dimethylohexyl, -1,3-dimethylohexyl, -3,3-dimethylohexyl, 1,2-dimethyloheptyl, -1,3-dimethyloheptyl, -3,3-dimethyloheptyl and the like.

The term “alkoxy” refers to an alkyl substituent as defined above bound by an oxygen atom.

The term “perfluoroalkyl” refers to an alkyl group as defined above in which all the hydrogen atoms have been substituted by the same or different halogen atoms.

The term “cycloalkyl” refers to a saturated mono- or polycyclic hydrocarbon substituent having the indicated number of carbon atoms. Examples of cycloalkyl substituents include -cyclopropyl, -cyclobutyl, -cyclopentyl, -cyclohexyl, -cycloheptyl, -cyclooctyl, -cyclononyl, -cyclodecyl and the like.

The term “alkenyl” refers to a non-saturated, linear or branched non-cyclic hydrocarbon substituent of the indicated number of carbon atoms and containing at least one double carbon-carbon bond. Examples of alkenyl substituents include -vinyl, -allyl, -1-butenyl, -2-butenyl, -isobutenyl, -1-pentenyl, -2-pentenyl, -3-methylo-1-butenyl, -2-methylo-2-butenyl, 2,3-dimethylo-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “cycloalkenyl” refers to a non-saturated mono- or polycyclic hydrocarbon substituent of the indicated number of carbon atoms and containing at least one double carbon-carbon bond. Examples of cycloalkenyl substituents include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl, -cyclononenyl, -cyclopentadienyl, -cyclodecenyl, -cyclodecadienyl and the like.

The term “alkinyl” refers to a non-saturated, linear or branched non-cyclic hydrocarbon substituent of the indicated number of carbon atoms and containing at least one triple carbon-carbon bond. Examples of alkynyl substituents include -acetylenyl, -propynyl, -1-butynyl, -2-butynyl, -1-pentynyl, -2-pentynyl, -3-methyl-1-butynyl, 4-pentynyl, -1-hexynyl, 2-hexynyl, -5-hexynyl and the like.

The term “cycloalkynyl” refers to a non-saturated mono- or polycyclic hydrocarbon substituent of the indicated number of carbon atoms and containing at least one triple carbon-carbon bond. Examples of cycloalkynyl substituents include -cyclohexyl, -cycloheptynyl, -cyclooctynyl and the like.

The term “aryl” refers to an aromatic mono- or polycyclic hydrocarbon substituent having the indicated number of carbon atoms. Examples of aryl substituents include phenyl, -tolyl, -xylyl, -naphthyl, -2,4,6-trimethylphenyl, -2-fluorophenyl, -4-fluorophenyl, -2,4,6-trifluorophenyl, -2,6-difluorophenyl, -4-nitrophenyl and the like.

The term “aralkyl” refers to an alkyl substituent as defined above substituted with at least one aryl as defined above. Examples of aralkyl substituents include -benzyl, -diphenylmethyl, -triphenylmethyl and the like.

The term “heteroaryl” refers to an aromatic mono- or polycyclic hydrocarbon substituent having the indicated number of carbon atoms, in which at least one carbon atom is substituted by a heteroatom selected from O, N and S atoms. Examples of heteroaryl substituents include -furyl, -thienyl, -imidazolyl, -oxazolyl, -thiazolyl, -isoxazolyl, -triazolyl, -oxadiazolyl, -thiadiazolyl, -tetrazolyl, -pyridyl, -pyrimidyl, -triazinyl, -indolyl, -benzo[b]furyl, -benzo[b]thienyl, -indazolyl, -benzoimidazolyl, -azaindolyl, -quinolyl, -isoquinolyl, -carbazolyl and the like.

The term “heterocycle” refers to a saturated or partially non-saturated, mono- or polycyclic hydrocarbon substituent having the indicated number of carbon atoms, in which at least one carbon atom is substituted by a heteroatom selected from O, N and S atoms. Examples of heterocyclic substituents include furyl, thiophenyl, pyrrolyl, oxazolyl, imidazolyl, thiazolyl, isoxazolyl, pyrazolyl, isothiazolyl, triazinyl, pyrrolidinonyl, pyrrolidinyl, hydantoinyl, oxiranyl, oxetanyl, tetrahydrofuranyl, tetrahydrothiophenyl, quinolinyl, isoquinolinyl, chromonyl, coumarinyl, indolyl, indolizinyl, benzo[b]furanyl, benzo[b]thiophenyl, indazolyl, purinyl, 4H-quinolizinyl, isoquinolyl, quinolyl, phthalazinyl, naphthyridinyl, carbazolyl, β-carbolinyl and the like.

The term “neutral ligand” refers to a non-charged substituent capable of coordinating with a metallic centre (the ruthenium atom). Examples of such ligands may include: amines, phosphines and oxides thereof, alkyl and aryl phosphites and phosphates, arsines and oxides thereof, ethers, alkyl and aryl sulphides, coordinated hydrocarbons, alkyl and aryl halides.

The term “anionic ligand” refers to a substituent capable of coordinating with a metallic centre (the ruthenium atom) with a charge capable of partially or completely compensating the charge of the metallic centre. Examples of such ligands may include fluoride, chloride, bromide, iodide, cyanide, cyanate and thiocyanate anions, carboxylic acid anions, alcohol anions, phenolic anions, thiol and thiophenol anions, delocalized charge hydrocarbon anions (e.g. cyclopentadiene), (organo)sulphuric and (organo)phosphoric acid anions and esters thereof (such as, for example, alkylsulphonic and aryl sulphonic acid anions, alkylphosphoric and arylphosphoric acid anions, sulphuric acid alkyl and aryl ester anions, phosphoric acid alkyl and aryl ester anions, alkylphosphoric and arylphosphoric alkyl and aryl ester anions).

Optionally, the anionic ligand may have interconnected L¹, L² and L³ groups, such as the catechol anion, the acetylacetone anion, the salicylaldehyde anion. Anionic ligands (X¹, X²) and neutral ligands (L¹, L², L³) may be interconnected to form multidentate ligands, such as a bidentate ligand (X¹—X²), a tridentate ligand (X¹—X²-L¹), a tetradentate ligand (X¹—X²-L¹-L²), a bidentate ligand (X¹-L¹), a tridentate ligand (X¹-L¹-L²), a tetradentate ligand (X¹-L¹-L²-L³), a bidentate ligand (L¹-L²), a tridentate ligand (L¹-L²-L³). Examples of such ligands include catechol anion, acetylacetone anion and salicylaldehyde anion.

The term “heteroatom” refers to an atom selected from the group comprising an atom of oxygen, sulphur, nitrogen, phosphorus and the like.

The term “chlorinated solvent” refers to a solvent, the structure of which comprises at least one atom of for example fluorine, chlorine, bromine and iodine; preferably more than one. Examples of such solvents include dichloromethane, chloroform, tetrachloromethane (carbon tetrachloride), 1,2-dichloroethane, chlorobenzene, perfluorobenzene, perfluorotoluene, freons and the like.

The term “organic non-polar solvent” refers to a solvent characterised by non-existent or very low dipole momentum. Examples of such solvents include pentane, hexane, octane, nonane, decane, benzene, toluene, xylene and the like.

The term “organic polar solvent” refers to a solvent characterised by a dipole momentum substantially greater than zero. Examples of such solvents include dimethylformamide (DMF), tetrahydrofuran (THF) and its derivatives, diethyl ether, dichloromethane, ethyl acetate, chloroform, alcohols (MeOH, EtOH or i-PrOH) and the like.

The term “GC” refers to gas chromatography.

The term “PAO” refers to poly-alpha-olefins, a group of polymers produced using alpha-olefins as monomers, i.e. alkenes containing a terminal double bond, i.e. between 1st and 2nd carbon atom.

Commercially available poly-alpha-olefins are designated with the abbreviation PAO and a number indicating the kinematic viscosity of the polymer at a temperature of 100° C.

The term “GCMS” denotes gas chromatography-mass spectrometry.

The term “HPLC” refers to high performance liquid chromatography, and solvents designated as “HPLC” solvents refer to solvents having sufficient purity for HPLC analysis.

The term “NMR” refers to nuclear magnetic resonance.

The term “NHC” refers to N-heterocycl carbene.

The term “precatalyst” refers to, in relation to ruthenium complexes, a 16-electron chemical compound which, after the step of dissociation of one ligand or reorganisation of the molecule, is converted to the 14-electron olefin metathesis catalyst as such, which is active in the catalytic cycle.

The term “TFQ” denotes tetrafluoro-1, 4-benzoquinone (CAS: 527-21-9).

The term “diene” as used in this patent document refers to substrates used for the macrocyclization reaction by way of RCM reaction. It is not strictly used, it rather refers to compounds where the pair of C═C double bonds co-reacts in the RCM reaction, resulting in a product, i.e. a cyclic olefin. Some Dx and Dy compounds herein may contain more than two C═C double bonds.

The phrase “substituted with at least one substituent” means that a group may be substituted with one substituents from those specified, two such substituents or more, up to the maximum number depending on the valency of the substituted atom, provided that such substitution results in a chemically stable molecule.

The term “effective efficiency” means the weighed yield of all resulting RCM macrocyclization products obtained in the reaction based on the expected macrocyclic compound. The effective yield, then determined in % based on the GC or GCMS chromatogram of the post-reaction mixture with respect to the expected product. The effective efficiency was used only for processes with low selectivity of the reaction. The low selectivity of the reaction is associated with the C═C double bond migration process, whereby the GC-MS chromatograph has registered a series of cyclic compounds differing by their mass±“n.CH₂”. Comparison of FIG. 5a and FIG. 5b.

### EMBODIMENTS OF THE INVENTION

The following examples are provided solely for the purpose of illustrating the invention and for clarifying the individual aspects thereof, and not with the intention to limit it, and should not be considered to be equivalent to the total scope thereof as defined in the appended claims. In the examples below, unless otherwise indicated, standard materials and methods were employed as used in the art or it was proceeded according to the manufacturer's recommendations for particular reagents and methods.

### Example I

General Method for the Preparation of Diene Esters (Dx), Substrates for the RCM Reaction

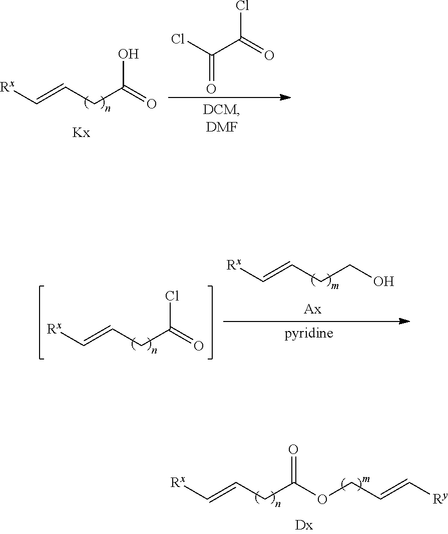

A suitable carboxylic acid K1, K2, K3 or K4 (1 eq.) was dissolved in anhydrous methylene chloride under an inert gas atmosphere. A few drops of N,N-dimethylformamide were added, and then oxalyl chloride (1.2 eq.) was added dropwise at room temperature. Gas emission and change of colour to yellow were observed. After an hour, substrate conversion was checked using ¹H NMR. Methylene chloride and unreacted oxalyl chloride were evaporated using a membrane pump. A new portion of solvent was added and the reaction mixture was cooled to a temperature of −78° C. Pyridine (2 eq.) was added dropwise, followed by suitable A1-A9 alcohol (0.9-1.1 eq.). The reaction was conducted for an hour, while monitoring the progress using thin-layer chromatography (TLC). The product was purified by column chromatography with 0.5% EtOAc/n-hexane (v/v).

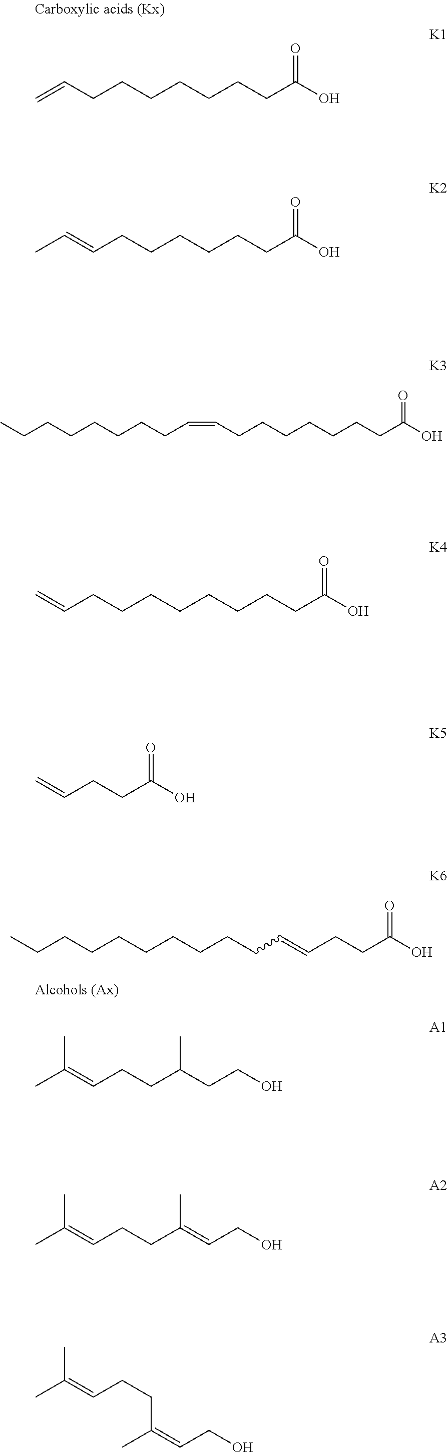

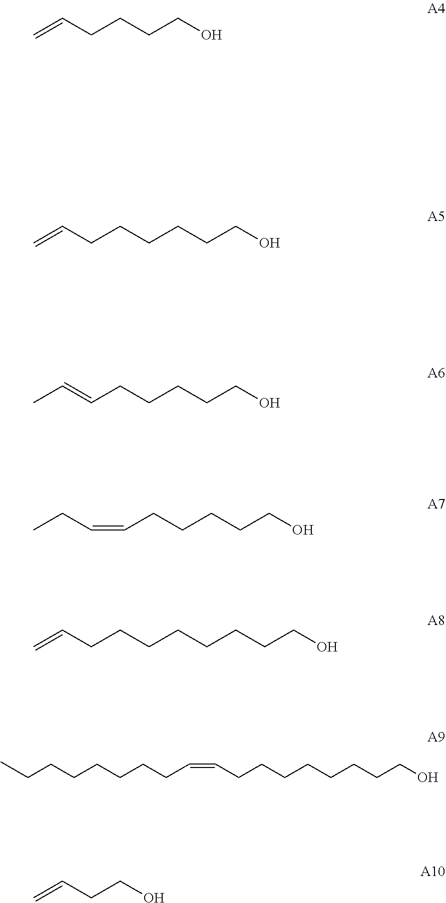

The procedure described above yielded the following dienes (Dx), substrates for the RCM reaction,

### 9-decanoic acid esters

### a) dec-9-en-1-yl dec-9-enoate (D1)

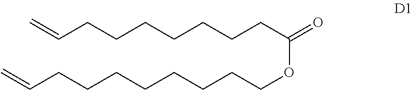 [SMILES: C=CCCCCCCCCOC(=O)CCCCCCCC=C]

¹H NMR (400 MHz, CDCl₃): δ (ppm)=5.77-5.64 (m, 2H), 4.93-4.79 (m, 4H), 3.95 (t, J=6.7 Hz, 2H), 2.19 (t, J=7.5 Hz, 2H), 1.97-1.88 (m, 4H), 1.56-1.48 (m, 4H), 1.30-1.16 (m, 18H).

¹³C NMR (101 MHz, CDCl₃): δ (ppm)=174.15 (CO), 139.30 (CH), 139.26 (CH), 114.33 (CH₂), 114.31 (CH₂), 64.54 (CH₂), 34.54 (CH₂), 33.92 (CH₂), 29.50 (CH₂), 29.34 (CH₂), 29.25 (CH₂), 29.24 (CH₂), 29.18 (CH₂), 29.08 (CH₂), 29.03 (CH₂), 28.99 (CH₂), 28.78 (CH₂), 26.06 (CH₂), 25.15 (CH₂).

### b) dec-9-enoate octo-7-en-1-yl (D2)

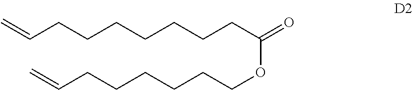 [SMILES: C=CCCCCCCCC(=O)OCCCCCCC=C]

¹H NMR (400 MHz, CDCl₃): δ (ppm)=5.86-5.74 (m, 2H), 5.03-4.89 (m, 4H), 4.05 (t, J=6.7 Hz, 2H), 2.33-2.23 (m, 2H), 2.09-1.98 (m, 4H), 1.68-1.55 (m, 4H), 1.44-1.23 (m, 14H).

¹³C NMR (101 MHz, CDCl₃): δ (ppm)=174.13 (CO), 139.27 (CH), 139.11 (CH), 114.46 (CH₂), 114.33 (CH₂), 64.49 (CH₂), 34.54 (CH₂), 33.91 (CH₂), 33.82 (CH₂), 29.26 (CH₂), 29.24 (CH₂), 29.08 (CH₂), 28.99 (CH₂), 28.91 (CH₂), 28.86 (CH₂), 28.74 (CH₂), 25.94 (CH₂), 25.14 (CH₂).

### c) dec-9-enoate hex-5-en-1-yl (D3)

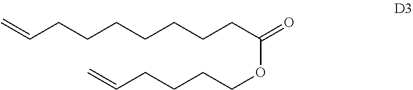 [SMILES: C=CCCCCCCCC(=O)OCCCCC=C]

¹H NMR (400 MHz, CDCl₃): δ (ppm)=5.86-5.74 (m, 2H), 5.05-4.89 (m, 4H), 4.07 (t, J=6.6 Hz, 2H), 2.29 (t, J=7.5 Hz, 2H), 2.14-1.98 (m, 4H), 1.70-1.54 (m, 4H), 1.52-1.22 (m, 10H).

¹³C NMR (101 MHz, CDCl₃): δ (ppm)=174.11 (CO), 139.27 (CH), 138.51 (CH), 114.94 (CH₂), 114.33 (CH₂), 64.30 (CH₂), 34.52 (CH₂), 33.91 (CH₂), 33.44 (CH₂), 29.25 (CH₂), 29.24 (CH₂), 29.07 (CH₂), 28.99 (CH₂), 28.23 (CH₂), 25.35 (CH₂), 25.13 (CH₂).

### d) dec-9-enoate cis-non-6-en-1-yl (D4)

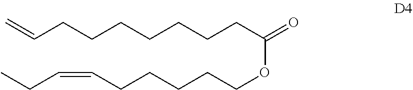 [SMILES: C=CCCCCCCCC(=O)OCCCCC/C=C\CC]

¹H NMR (400 MHz, CDCl₃): δ (ppm)=5.86-5.74 (m, 1H), 5.41-5.26 (m, 2H), 5.04-4.90 (m, 2H), 4.05 (t, J=6.7 Hz, 2H), 2.29 (t, J=7.5 Hz, 2H), 2.08-1.98 (m, 6H), 1.70-1.57 (m, 4H), 1.44-1.25 (m, 12H), 0.96 (t, J=7.5 Hz, 3H).

¹³C NMR (101 MHz, CDCl₃): δ (ppm)=174.12 (CO), 139.27 (CH), 132.00 (CH), 128.97 (CH), 114.33 (CH₂), 64.48 (CH₂), 34.53 (CH₂), 33.91 (CH₂), 29.49 (CH₂), 29.26 (CH₂), 29.24 (CH₂), 29.07 (CH₂), 28.99 (CH₂), 28.71 (CH₂), 27.07 (CH₂), 25.71 (CH₂), 25.14 (CH₂), 20.67 (CH₂), 14.53 (CH₃).

### e) dec-9-enoate 3,7-dimethyl-octo-6-en-1-yl (D5)

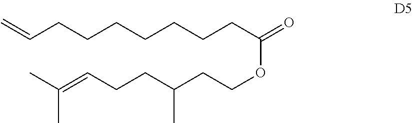 [SMILES: C=CCCCCCCCC(=O)OCCC(C)CCC=C(C)C]

¹H NMR (400 MHz, CDCl₃): δ (ppm)=5.87-5.73 (m, 1H), 5.08 (m, 1H), 5.03-4.88 (m, 2H), 4.26-4.01 (m, 2H), 2.35-2.23 (m, 2H), 2.11-1.89 (m, 4H), 1.73-1.10 (m, 20H), 0.91 (d, J=7.5 Hz, 3H).

¹³C NMR (101 MHz, CDCl₃): δ (ppm)=174.12 (CO), 139.27 (CH), 131.47 (C), 124.71 (CH), 114.33 (CH₂), 62.91 (CH₂), 37.13 (CH₂), 35.62 (CH₂), 34.56 (CH₂), 33.91 (CH₂), 29.63 (CH), 29.25 (CH₂), 29.08 (CH₂), 28.99 (CH₂), 25.88 (CH₃), 25.54 (CH₂), 25.14 (CH₂), 19.56 (CH₃), 17.81 (CH₃).

### f) dec-9-enoatetrans-3,7-dimethylocto-2, 6-dien-1-yl(D6)

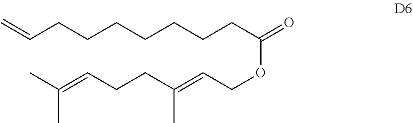 [SMILES: C=CCCCCCCCC(=O)OC/C=C(\C)CCC=C(C)C]

¹H NMR (400 MHz, CDCl₃): δ (ppm)=5.87-5.73 (m, 1H), 5.38-5.28 (m, 1H), 5.13-5.04 (m, 1H), 5.03-4.86 (m, 2H), 4.59 (d, J=7.1 Hz, 2H), 2.33-2.25 (m, 2H), 2.15-1.99 (m, 6H), 1.69 (dd, J=7.9, 0.8 Hz, 6H), 1.66-1.54 (m, 5H), 1.42-1.24 (m, 8H).

¹³C NMR (101 MHz, CDCl₃): δ (ppm)=174.05 (CO), 142.27 (C), 139.27 (CH), 131.97 (C), 123.89 (CH), 118.52 (CH), 114.32 (CH₂), 61.33 (CH₂), 39.68 (CH₂), 34.52 (CH₂), 33.92 (CH₂), 29.25 (CH₂), 29.23 (CH₂), 29.07 (CH₂), 28.99 (CH₂), 26.44 (CH₂), 25.84 (CH₃), 25.13 (CH₂), 17.84 (CH₃), 16.62 (CH₃).

### g) dec-9-enoate cis-3,7-dimethylocto-2, 6-dien-1-yl (D7)

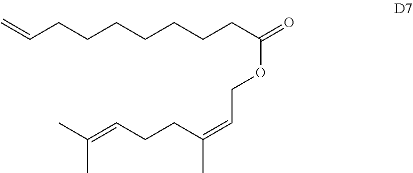 [SMILES: C=CCCCCCCCC(=O)OC/C=C(/C)CCC=C(C)C]

¹H NMR (400 MHz, CDCl₃): δ (ppm)=5.87-5.73 (m, 1H), 5.38-5.28 (m, 1H), 5.13-5.04 (m, 1H), 5.03-4.86 (m, 2H), 4.56 (dd, J=7.3, 0.8 Hz, 2H), 2.33-2.25 (m, 2H), 2.15-1.99 (m, 6H), 1.76 (d, J=1.3 Hz, 3H), 1.70-1.57 (m, 8H), 1.42-1.24 (m, 8H).

¹³C NMR (101 MHz, CDCl₃): δ (ppm)=174.03 (CO), 142.65 (C), 139.28 (CH) 132.30 (C), 123.72 (CH) 119.39 (CH), 114.32 (CH₂), 61.05 (CH₂), 34.53 (CH₂), 33.91 (CH₂), 32.31 (CH₂), 29.25 (CH₂), 29.07 (CH₂), 28.99 (CH₂), 26.82 (CH₂) 25.85 (CH₃) 25.12 (CH₂), 23.68 (CH₃), 17.81 (CH₃).

### h) oleyl dec-9-enoate (D8)

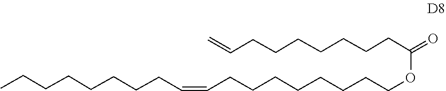 [SMILES: C=CCCCCCCCC(=O)OCCCCCCCC/C=C\CCCCCCCC]

¹H NMR (400 MHz, CDCl₃): δ (ppm)=5.87-5.72 (m, 1H), 5.41-5.27 (m, 2H), 5.04-4.89 (m, 2H), 4.05 (t, J=6.7 Hz, 2H), 2.28 (t, J=7.5 Hz, 2H), 2.08-1.97 (m, 6H), 1.50-1.16 (m, 30H), 0.88 (t, J=6.5 Hz, 3H).

¹³C NMR (101 MHz, CDCl₃): δ (ppm)=169.87, 145.71, 139.23, 139.07, 114.47, 114.34, 101.82, 53.85, 33.89, 33.84, 29.45, 29.17, 29.03, 28.98, 28.97, 28.88, 28.87, 27.62, 26.43, 24.75.

Oleic Acid Esters

### a) dec-9-en-1-yl oleinate (D9)

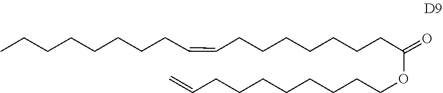 [SMILES: C=CCCCCCCCCOC(=O)CCCCCCC/C=C\CCCCCCCC]

¹H NMR (400 MHz, CDCl₃): δ (ppm)=5.86-5.74 (m, 1H), 5.41-−5.26 (m, 2H), 5.05-4.90 (m, 2H), 4.05 (t, J=6.7 Hz, 2H), 2.28 (t, J=7.5 Hz, 2H), 2.06-1.94 (m, 6H), 1.49-1.17 (m, 30H), 0.88 (t, J=6.5 Hz, 3H).

¹³C NMR (101 MHz, CDCl₃): δ (ppm)=174.15, 139.30, 130.13, 129.89, 114.31, 64.53, 34.55, 33.94, 32.06, 29.92, 29.84, 29.68, 29.50, 29.48, 29.34, 29.33, 29.29, 29.26, 29.18, 29.03, 28.78, 27.37, 27.31, 26.06, 25.16, 22.84, 14.28.

### b) octo-7-en-1-yl oleate (D10)

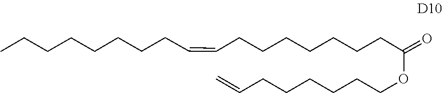 [SMILES: C=CCCCCCCOC(=O)CCCCCCC/C=C\CCCCCCCC]

¹H NMR (400 MHz, CDCl₃): δ (ppm)=5.89-5.71 (m, 1H), 5.43-5.27 (m, 2H), 5.10-4.86 (m, 2H), 4.05 (t, J=6.7 Hz, 2H), 2.29 (t, J=7.5 Hz, 2H), 1.47-1.19 (m, 24H), 0.97-0.81 (m, 3H).

¹³C NMR (101 MHz, CDCl₃): δ (ppm)=174.14, 139.11, 130.14, 129.90, 114.46, 64.49, 34.55, 33.83, 32.06, 29.93, 29.85, 29.68, 29.48, 29.33, 29.30, 29.27, 28.92, 28.86, 28.75, 27.37, 27.32, 25.94, 25.17, 22.84, 14.28.

### c) hex-5-en-1-yl oleate (D11)

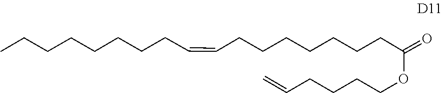 [SMILES: C=CCCCCOC(=O)CCCCCCC/C=C\CCCCCCCC]

¹H NMR (400 MHz, CDCl₃): δ (ppm)=5.80 (ddt, J=16.9, 10.2, 6.7 Hz, 1H), 5.42-5.27 (m, 2H), 5.09-4.90 (m, 2H), 4.07 (t, J=6.6 Hz, 2H), 2.29 (t, J=7.4 Hz, 2H), 2.14-1.96 (m, 4H), 1.70-1.57 (m, 4H), 1.52-1.39 (m, 2H), 1.38-1.21 (m, 22H), 0.92-0.84 (m, 3H).

¹³C NMR (101 MHz, CDCl₃): δ (ppm)=174.12, 138.51, 130.14, 129.89, 114.95, 64.30, 34.53, 33.44, 32.06, 29.92, 29.85, 29.68, 29.48, 29.33, 29.29, 29.26, 28.23, 27.37, 27.32, 25.35, 25.16, 22.84, 14.28.

### d) cis-non-6-en-1-yl oleate (D12)

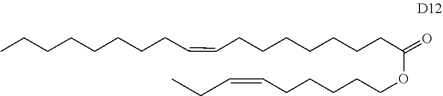 [SMILES: CC/C=C\CCCCCOC(=O)CCCCCCC/C=C\CCCCCCCC]

¹H NMR (400 MHz, CDCl₃): δ (ppm)=5.45-5.24 (m, 4H), 4.05 (t, J=6.7 Hz, 2H), 2.36-2.23 (m, 2H), 2.11-1.92 (m, 8H), 1.69-1.57 (m, 4H), 1.45-1.22 (m, 24H), 0.95 (t, J=7.6 Hz, 3H), 0.90-0.86 (m, 3H).

¹³C NMR (101 MHz, CDCl₃): δ (ppm)=174.13, 132.01, 130.14, 129.90, 128.97, 64.48, 34.55, 32.06, 29.92, 29.85, 29.68, 29.50, 29.48, 29.34, 29.29, 29.27, 28.71, 27.37, 27.32, 27.08, 25.71, 25.17, 22.84, 20.67, 14.53, 14.28.

### e) 3,7-dimethyl-octo-6-en-1-yl oleate (D13)

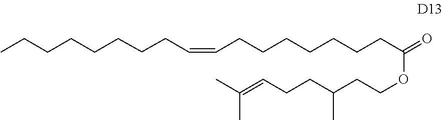 [SMILES: CCCCCCCC/C=C\CCCCCCCC(=O)OCCC(C)CCC=C(C)C]

¹H NMR (400 MHz, CDCl₃): δ (ppm)=5.43-5.26 (m, 2H), 5.08 (tₛₑₚₜₑₜₛ, J=7.1, 1.4 Hz, 1H), 4.16-4.03 (m, 2H), 2.28 (t, J=7.6 Hz, 2H), 2.07-1.88 (m, 6H), 1.70-1.66 (m, 3H), 1.61-1.59 (m, 3H), 1.72-1.38 (m, 6H), 1.37-1.23 (m, 20H), 1.23-1.12 (m, 1H), 0.97-0.82 (m, 6H).

¹³C NMR (101 MHz, CDCl₃): δ (ppm)=174.13, 131.47, 130.13, 129.89, 124.71, 62.92, 37.13, 35.62, 34.57, 32.06, 29.92, 29.85, 29.68, 29.63, 29.48, 29.33, 29.29, 29.27, 27.37, 27.32, 25.88, 25.54, 25.16, 22.84, 19.56, 17.80, 17.80, 14.28.

### f) trans-3,7-dimethylocto-2, 6-dien-1-yl oleate (D14)

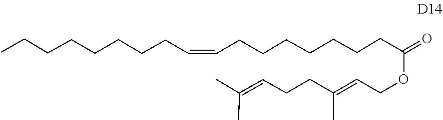 [SMILES: CCCCCCCC/C=C\CCCCCCCC(=O)OC/C=C(\C)CCC=C(C)C]

¹H NMR (400 MHz, CDCl₃): δ (ppm)=5.40-5.26 (m, 3H), 5.12-5.05 (m, 1H), 4.59 (d, J=7.1 Hz, 2H), 2.29 (t, J=7.6 Hz, 2H), 2.14-1.94 (m, 8H), 1.73-1.66 (m, 6H), 1.66-1.55 (m, 6H), 1.39-1.21 (m, 20H), 0.93-0.83 (m, 3H).

¹³C NMR (101 MHz, CDCl₃): δ (ppm)=174.06, 142.27, 131.96, 130.13, 129.90, 123.89, 118.52, 61.33, 39.68, 34.54, 32.06, 29.92, 29.85, 29.68, 29.48, 29.33, 29.28, 29.26, 27.37, 27.32, 26.44, 25.84, 25.16, 22.84, 17.84, 16.62, 14.28.

### g) cis-3,7-dimethylocto-2, 6-dien-1-yl oleate (D15

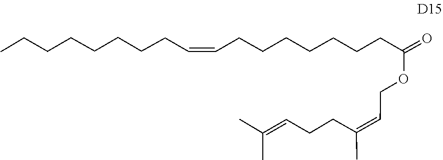 [SMILES: CCCCCCCC/C=C\CCCCCCCC(=O)OC/C=C(/C)CCC=C(C)C]

¹H NMR (400 MHz, CDCl₃): δ (ppm)=5.40-5.27 (m, 3H), 5.13-5.05 (m, 1H), 4.59-4.52 (m, 2H), 2.29 (t, J=7.6 Hz, 2H), 2.16-1.94 (m, 8H), 1.76 (q, J=1.1 Hz, 3H), 1.71-1.65 (m, 3H), 1.67-1.54 (m, 5H), 1.38-1.21 (m, 20H), 0.92-0.83 (m, 3H).

¹³C NMR (101 MHz, CDCl₃): δ (ppm)=174.05, 142.64, 132.30, 130.13, 129.90, 123.72, 119.39, 61.05, 34.54, 32.31, 32.06, 29.92, 29.84, 29.68, 29.48, 29.33, 29.29, 29.26, 27.36, 27.32, 26.81, 25.85, 25.14, 23.68, 22.84, 17.82, 14.28.

### h) Oleyl Oleate (D16)

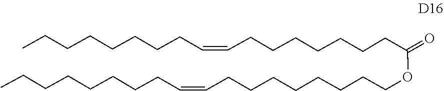 [SMILES: CCCCCCCC/C=C\CCCCCCCCOC(=O)CCCCCCC/C=C\CCCCCCCC]

¹H NMR (400 MHz, CDCl₃): δ (ppm)=5.40-5.30 (m, 4H), 4.05 (t, J=6.7 Hz, 2H), 2.29 (t, J=7.6 Hz, 2H), 2.06-1.96 (m, 8H), 1.68-1.56 (m, 4H), 1.40-1.20 (m, 42H), 0.88 (t, J=6.8 Hz, 6H).

¹³C NMR (101 MHz, CDCl₃): δ (ppm)=174.15, 130.14, 130.12, 129.93, 129.90, 124.73, 64.55, 34.55, 32.06, 29.92, 29.89, 29.85, 29.68, 29.58, 29.48, 29.39, 29.37, 29.33, 29.29, 29.27, 28.80, 27.37, 27.34, 27.32, 26.08, 25.17, 22.85, 14.28.

Esters of 9-Decenoic Acid Following Isomerization of Double Bonds

### a) dec-8-en-1-yl dec-8-enoate (D17)

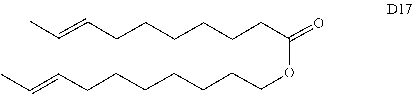 [SMILES: C/C=C/CCCCCCCOC(=O)CCCCCC/C=C/C]

¹H NMR (400 MHz, (CD₃)₂CO): δ (ppm)=5.48-5.32 (m, 4H), 4.02 (t, 2H, J=6.7 Hz), 2.27 (t, 2H, J=7.4 Hz), 2.01-1.91 (m, 4H), 1.71-1.54 (m, 10H), 1.38-1.12 (m, 14H).

¹³C NMR (100 MHz, (CD₃)₂CO): δ (ppm)=173.4 (CO), 132.1 (CH), 132.1 (CH), 125.2 (CH), 125.1 (CH), 64.4 (CH₂), 34.5 (CH₂), 33.2 (CH₂), 33.1 (CH₂), 30.2 (CH₂), 30.1 (CH₂), 29.8 (CH₂), 29.7 (CH₂), 29.6 (CH₂), 29.5 (CH₂), 29.4 (CH₂), 26.6 (CH₂), 25.6 (CH₂), 18.0 (2×CH₃).

### b) cis-non-6-en-1-yl dec-8-enoate (D18)

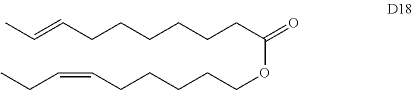 [SMILES: C/C=C/CCCCCCC(=O)OCCCCC/C=C\CC]

¹H NMR (400 MHz, (CD₃)₂CO): δ (ppm)=5.46-5.27 (m, 4H), 4.03 (t, 2H, J=6.6 Hz), 2.27 (t, 2H, J=7.4 Hz), 2.07-1.94 (m, 6H), 1.63-1.56 (m, 7H), 1.38-1.30 (m, 10H), 0.93 (t, 3H, J=7.6 Hz).

¹³C NMR (100 MHz, (CD₃)₂CO): δ (ppm)=173.5 (CO), 132.3 (CH), 132.1 (CH), 129.6 (CH), 125.2 (CH), 64.4 (CH₂), 34.6 (CH₂), 33.2 (CH₂), 30.1 (CH₂), 30.0 (CH₂), 29.6 (CH₂), 29.5 (CH₂), 29.3 (CH₂), 27.5 (CH₂), 26.2 (CH₂), 25.6 (CH₂), 21.0 (CH₂), 18.0 (CH₃), 14.6 (CH₃).

### c) octo-6-en-1-yl dec-8-enoate (D19)

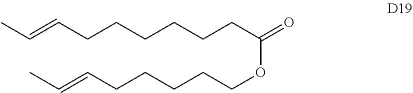 [SMILES: C/C=C/CCCCCCC(=O)OCCCCC/C=C/C]

¹H NMR (400 MHz, (CD₃)₂CO): δ (ppm)=5.42-5.39 (m, 4H), 4.02 (t, 2H, J=6.6 Hz), 2.26 (t, 2H, J=7.4 Hz), 2.00-1.93 (m, 4H), 1.66-1.54 (m, 10H), 1.39-1.29 (m, 10H).

¹³C NMR (100 MHz, (CD₃)₂CO): δ (ppm)=173.5 (CO), 132.1 (CH), 132.0 (CH), 125.3 (CH), 125.2 (CH), 64.4 (CH₂), 34.6 (CH₂), 33.1 (CH₂), 33.1 (CH₂), 30.1 (CH₂), 29.9 (CH₂), 29.6 (CH₂), 29.5 (CH₂), 29.3 (CH₂), 26.1 (CH₂), 25.6 (CH₂), 18.0 (2×CH₃).

### d) dec-9-en-1-yl dec-8-enoate (D20)

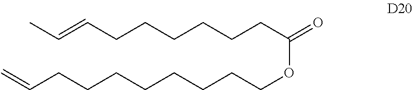 [SMILES: C=CCCCCCCCCOC(=O)CCCCCC/C=C/C]

¹H NMR (400 MHz, (CDCl₃): δ (ppm)=5.91-5.69 (m, 1H), 5.54-5.30 (m, 2H), 5.08-4.86 (m, 2H), 4.05 (t, J=6.7 Hz, 2H), 2.37-2.24 (m, 2H), 2.09-2.00 (m, 2H), 2.00-1.91 (m, 2H), 1.70-1.53 (m, 8H), 1.45-1.21 (m, 13H).

¹³C NMR (100 MHz, CDCl₃): δ (ppm)=174.14 (CO), 139.30 (CH), 131.60 (CH), 124.85 (CH), 114.30 (CH₂), 64.54 (CH₂), 34.55 (CH₂), 33.93 (CH₂), 32.65 (CH₂), 29.54 (CH₂), 29.50 (CH₂), 29.34 (CH₂), 29.18 (CH₂), 29.17 (CH₂), 29.03 (CH₂), 28.92 (CH₂), 28.79 (CH₂), 26.06 (CH₂), 25.14 (CH₂), 18.07 (CH₂).

### e) octo-7-en-1-yl dec-8-enoate (D21)

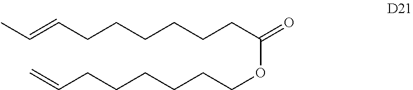 [SMILES: C=CCCCCCCOC(=O)CCCCCC/C=C/C]

¹H NMR (400 MHz, CDCl₃): δ (ppm)=5.80 (ddt, J=17.0, 10.2, 6.7 Hz, 1H), 5.58-5.30 (m, 2H), 5.09-4.86 (m, 2H), 4.05 (t, J=6.7 Hz, 2H), 2.28 (t, J=7.5 Hz, 2H), 2.09-2.00 (m, 2H), 1.99-1.91 (m, 2H), 1.70-1.57 (m, 6H), 1.46-1.22 (m, 13H).

¹³C NMR (100 MHz, CDCl₃): δ (ppm)=174.13 (CO), 139.10 (CH), 131.59 (CH), 124.85 (CH), 114.45 (CH₂), 64.48 (CH₂), 34.54 (CH₂), 33.82 (CH₂), 32.64 (CH₂), 29.53 (CH₂), 29.17 (CH₂), 28.91 (CH₂), 28.85 (CH₂), 28.75 (CH₂), 25.94 (CH₂), 25.13 (CH₂), 18.07 (CH₂).

### f) octo-6-en-1-yl dec-9-enoate (D22)

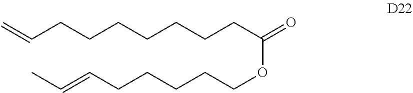 [SMILES: C=CCCCCCCCC(=O)OCCCCC/C=C/C]

¹H NMR (400 MHz, CDCl₃): δ (ppm)=5.80 (ddt, J=16.9, 10.2, 6.7 Hz, 1H), 5.48-5.33 (m, 2H), 5.13-4.84 (m, 2H), 4.05 (t, J=6.7 Hz, 2H), 2.29 (t, J=7.5 Hz, 2H), 2.12-1.92 (m, 4H), 1.71-1.56 (m, 6H), 1.46-1.24 (m, 13H).

¹³C NMR (100 MHz, CDCl₃): δ (ppm)=174.11 (CO), 139.26 (CH), 131.32 (CH), 125.07 (CH), 114.33 (CH₂), 64.48 (CH₂), 34.54 (CH₂), 33.91 (CH₂), 32.56 (CH₂), 29.31 (CH₂), 29.25 (CH₂), 29.24 (CH₂), 29.08 (CH₂), 28.99 (CH₂), 28.67 (CH₂), 25.58 (CH₂), 25.14 (CH₂), 18.07 (CH₃).

### g) hex-5-en-1-yl undec-10-enoate (D23)

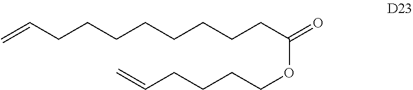 [SMILES: C=CCCCCCCCCC(=O)OCCCCC=C]

¹H NMR (400 MHz, CDCl₃): δ (ppm)=5.80 (ddtd, J=16.9, 10.1, 6.7, 4.3 Hz, 2H), 5.07-4.86 (m, 4H), 4.06 (t, J=6.6 Hz, 2H), 2.32-2.25 (m, 2H), 2.14-1.95 (m, 4H), 1.73-1.54 (m, 4H), 1.50-1.41 (m, 2H), 1.40-1.25 (m, 10H).

¹³C NMR (100 MHz, CDCl₃): δ (ppm)=174.13 (CO), 139.33 (CH), 138.51 (CH), 114.94 (CH₂), 114.28 (CH₂), 64.29 (CH₂), 34.52 (CH₂), 33.94 (CH₂), 33.44 (CH₂), 29.44 (CH₂), 29.35 (CH₂), 29.28 (CH₂), 29.20 (CH₂), 29.03 (CH₂), 28.22 (CH₂), 25.35 (CH₂), 25.14 (CH₂).

### Example II

An Alternative Esterification Reaction Yielding Dienes Used in the Metathesis Cyclization RCM (PRF=Porous Phenolsulfonic Acid-Formaldehyde Resin Catalyst).

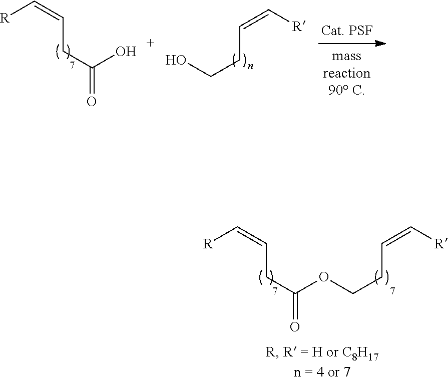

Carboxylic acid (1 eq.), alcohol (1 eq.) and a PRF (porous phenolsulfonic acid formaldehyde resin) catalyst (10 mg/l mmol acid) were placed in a single-necked round flask with a stirring element, prepared according to the procedure known from literature [M. Minakawa, H. Baek, Y. M. A. Yamada, J. W. Han, Y. Uozumi, Org. Lett., 2013, 15, 5798-5801]). The reaction flask was then sealed with a glass stopper and placed in an oil bath at a temperature of 90° C. for 14 hours. Afterwards, the reaction mixture was cooled to room temperature and diluted with n-heptane approx. tenfold. After filtering off the catalyst, the mixture was concentrated under reduced pressure and the crude product was purified by column chromatography using silica gel and 1% EtOAc/n-heptane mixture (v/v) as eluent.

### a) Oleyl Oleate (D16)

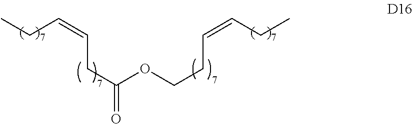

Obtained according to the general procedure using: oleic acid (0.72 g, 2.6 mmol), oleic alcohol (0.68 g, 2.6 mmol) and PSF (26 mg). 92% yield (1.15 g).

¹H NMR (400 MHz, CDCl₃) δ (ppm)=5.41-5.24 (m, 4H), 4.05 (t, J=6.7 Hz, 2H), 2.29 (t, J=7.5 Hz, 2H), 2.09-1.90 (m, 8H), 1.73-1.49 (m, 4H), 1.39-1.20 (m, 42H), 0.88 (t, J=6.8 Hz, 6H) ppm

¹³C NMR (101 MHz, CDCl₃): δ (ppm)=174.14, 130.13, 130.11, 129.92, 129.89, 64.54, 34.55, 32.06, 29.92, 29.88, 29.85, 29.68, 29.58, 29.48, 29.39, 29.37, 29.33, 29.29, 29.27, 28.80, 27.37, 27.34, 27.32, 26.08, 25.17, 22.84, 14.28 ppm.

### b) Hept-6-en-1-yl oleate (D24)

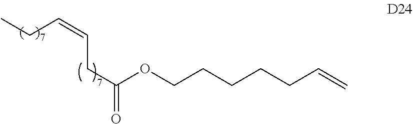

Obtained according to the general procedure using: oleic acid (1.26 g, 4.4 mmol), hept-6-en-1-ol (0.50 g, 4.4 mmol). 59% yield (0.99 g).

¹H NMR (400 MHz, CDCl₃) δ (ppm)=5.80 (ddt, J=16.9, 10.2, 6.7 Hz, 1H), 5.43-5.25 (m, 2H), 5.06-4.87 (m, 2H), 4.06 (t, J=6.7 Hz, 2H), 2.35-2.21 (m, 2H), 2.11-1.96 (m, 6H), 1.71-1.54 (m, 4H), 1.48-1.19 (m, 24H), 0.88 (t, J=6.9 Hz, 3H).

¹³C NMR (101 MHz, CDCl₃) δ (ppm)=174.12, 138.86, 130.13, 129.89, 114.62, 64.42, 34.54, 33.76, 32.06, 29.92, 29.84, 29.68, 29.48, 29.33, 29.29, 29.26, 28.65, 28.63, 27.37, 27.32, 25.56, 25.16, 22.84, 14.28.

### c) Dec-9-enoate cis-non-6-en-1-yl (D4)

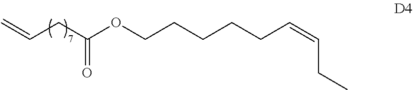

The reaction time was 20 hours, otherwise the conditions were as in the general procedure. Quantity of reagents: dec-9-enoic acid (1.19 g, 7.0 mmol), cis-non-6-en-1-ol (1.00 g, 7.0 mmol), PSF (70 mg). 85% yield (1.74 g).

¹H NMR (400 MHz, CDCl₃) δ (ppm)=5.80 (ddtd, J=16.9, 10.2, 6.7, 1.8 Hz, 2H), 5.05-4.86 (m, 4H), 4.05 (t, J=6.7 Hz, 2H), 2.28 (t, J=7.5 Hz, 2H), 2.14-1.97 (m, 4H), 1.68-1.53 (m, 4H), 1.49-1.22 (m, 12H).

¹³C NMR (101 MHz, CDCl₃) δ (ppm)=174.10, 139.24, 138.84, 114.61, 114.32, 77.48, 77.16, 77.16, 76.84, 64.42, 34.51, 33.90, 33.75, 33.74, 29.24, 29.23, 29.06, 28.98, 28.63, 28.62, 25.55, 25.13.

### d) Hept-6-en-1-yl dec-9-enoate (D25)

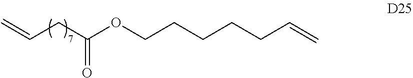

The reaction time was 66 hours, otherwise the conditions were as in the general procedure. Quantities of reagents: dec-9-enoic acid (1.49 g, 8.8 mmol), hept-6-en-1-ol (1.00 g, 8.8 mmol), PSF (90 mg). 57% yield (1.33 g).

¹H NMR (400 MHz, CDCl₃) δ (ppm)=5.80 (ddt, J=17.0, 10.2, 6.7 Hz, 1H), 5.45-5.24 (m, 2H), 5.05-4.85 (m, 2H), 4.05 (t, J=6.7 Hz, 2H), 2.28 (t, J=7.6 Hz, 2H), 2.11-1.95 (m, 6H), 1.67-1.54 (m, 4H), 1.48-1.22 (m, 12H), 0.95 (t, J=7.5 Hz, 3H).

¹³C NMR (101 MHz, CDCl₃) δ (ppm)=174.11, 139.25, 131.99, 128.96, 114.32, 77.48, 77.16, 76.84, 64.47, 34.52, 33.91, 29.48, 29.25, 29.24, 29.07, 28.98, 28.70, 27.07, 25.70, 25.14, 20.66, 14.52.

### Example III

Description of the etheral diene synthesis, (9Z)-1-(((6Z)-non-6-en-1-yl)oxy)octadeca-9-en (D26)

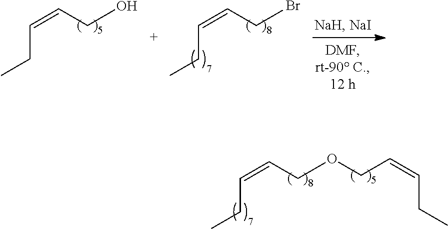

Under an inert gas atmosphere, alcohol A7 (1 eq.) was dissolved in anhydrous N,N-dimethylformamide. Sodium hydride (1.2 eq.) was added in 4 portions and stirred at room temperature for 30 minutes, then the temperature was increased to 50° C. and the stirring continued for another 30 minutes. One portion of sodium iodide was then added. Finally, oleyl bromide was added dropwise and the temperature was increased to 90° C. The reaction was continued under argon atmosphere for 12 hours. The crude mixture was washed with water (3×10 mL) and brine (1×10 mL) and dried over sodium sulfate (Na₂SO₄). The product was purified by column chromatography with 2% EtOAc/n-hexane (v/v). The target compound D26 was obtained at a 53% yield.

### (9Z)-1-(((6Z)-non-6-en-1-yl)oxy)octadeca-9-en (D26)

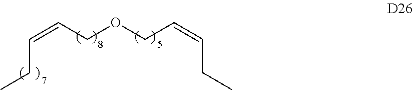

¹H NMR (400 MHz, CDCl₃) δ (ppm)=5.43-5.28 (m, 4H), 3.39 (dt, J=6.8, 0.9 Hz, 4H), 2.08-1.94 (m, 8H), 1.62-1.50 (m, 4H), 1.42-1.19 (m, 26H), 0.95 (t, J=7.5 Hz, 3H), 0.91-0.85 (m, 3H).

¹³C NMR (101 MHz, CDCl₃) δ (ppm)=131.80, 130.07, 130.00, 129.25, 71.13, 71.04, 32.06, 29.93, 29.91, 29.84, 29.78, 29.68, 29.66, 29.65, 29.48, 29.42, 27.36, 27.20, 26.35, 26.00, 22.85, 20.66, 14.55, 14.29.

### Example IV

General Procedure for the RCM Macrocyclization Reaction in Toluene at Atmospheric Pressure in the Presence of an Inert Gas. Preparation of One Compound M11 from Four Different Dienes D1, D8, D9 and D16.

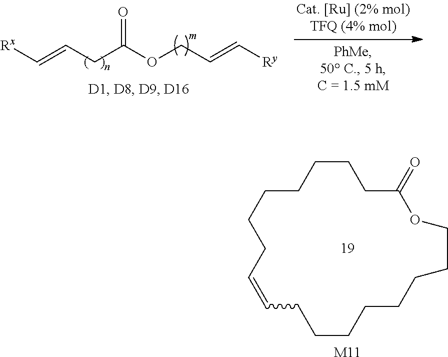

Suitable diene Dx (1 eq.) and tetrafluorobenzoquinone (4 mol %) were dissolved in anhydrous toluene (concentration of 1.5 mM) in a dried flask under inert gas atmosphere. Precatalyst (Cat. 1, 2 mol %) was dissolved in dry toluene in a Schlenk flask (concentration of 1 mg/l mL) and added during the reaction at intervals of 15 minutes (20 portions of the catalyst). The reaction was conducted at atmospheric pressure under inert gas atmosphere (argon). The reaction was conducted at 50° C. for 5 hours. The progress of the reaction was monitored using thin layer chromatography (TLC). Once the reaction was over, the catalyst was deactivated by the addition of SnatchCat solution [CAS: 51641-96-4] and stirred for 30 minutes. The solvent was evaporated, and the product was purified by column chromatography with 20% toluene/n-hexane (v/v).

RCM Reactions of ester dienes, derivatives of oleic and 9-decenoic acid in toluene.

The purpose of the example is to demonstrate the possibility of synthesis of macrocyclic lactones from biomass: oleic and 9-decenoic acids. In addition, there occurs a non-terminal double bond C═C—ester derivatives with one non-terminal double bond are closed in RCM reactions, achieving higher yields of the expected products. Preparation of one type of a macrocyclic compound (M11) from four different dienes (Dx)—for details, refer to FIG. 2

Reaction Conditions:
Solvent: anhydrous toluene [PhMe]
Reaction time: 5 hours
Temperature: 50° C.
Additives: tetrafluorobenzoquinone (4 mol %)
Precatalyst [Ru]: Cat. 1 (2% mol)
Substrate concentration: 0.0015 mol/dm³ [C=1.5 mM]

TABLE 1
Results of the preparation reaction of
19-membered M11 macrocycle.

| Item | Substrate | Product | Efficacy [%] |
|---|---|---|---|
| 1. | D1 | M11 | 77 |
| 2. | D8 | M11 | 92 |
| 3. | D9 | M11 | 90 |
| 4. | D16 | M11 | 38 |

The graphical presentation of the experiments is shown in FIG. 2

### Example V

Preparation of 16- and 17-Membered Macrocycles in the RCM Reaction in Toluene, Atmospheric Pressure, Inert Gas Atmosphere.

This example uses the same reaction conditions as in example III for a wider spectrum of dienes (Dx). The cumulative results are presented in Table 2 below. The graphical presentation of the experiments is included in FIG. 3.

TABLE 2
Results of the preparation reaction of 17-
membered and 16-membered macrocycles.

| Item | Substrate | Product | Efficacy [%] |
|---|---|---|---|
| 1a. | D3 | M1 | 72 |
| 1b. | D11 | M1 | 62 |
| 2a. | D4 | M3 | 87 |
| 2b. | D12 | M3 | 91 |
| 3a. | D5 | M5 | 88 |
| 3b. | D13 | M5 | 48 |
| 4a. | D7 | M6 | 87 |
| 4b. | D15 | M6 | 40 |
| 5a. | D6 | M7 | 80 |
| 5b. | D16 | M7 | 48 |
| 6a. | D2 | M9 | 77 |
| 6b. | D10 | M9 | 90 |

### Example VI

General Method for the RCM Reactions of Dienes with C═C Bonds Substituted with Short Alkyl Groups

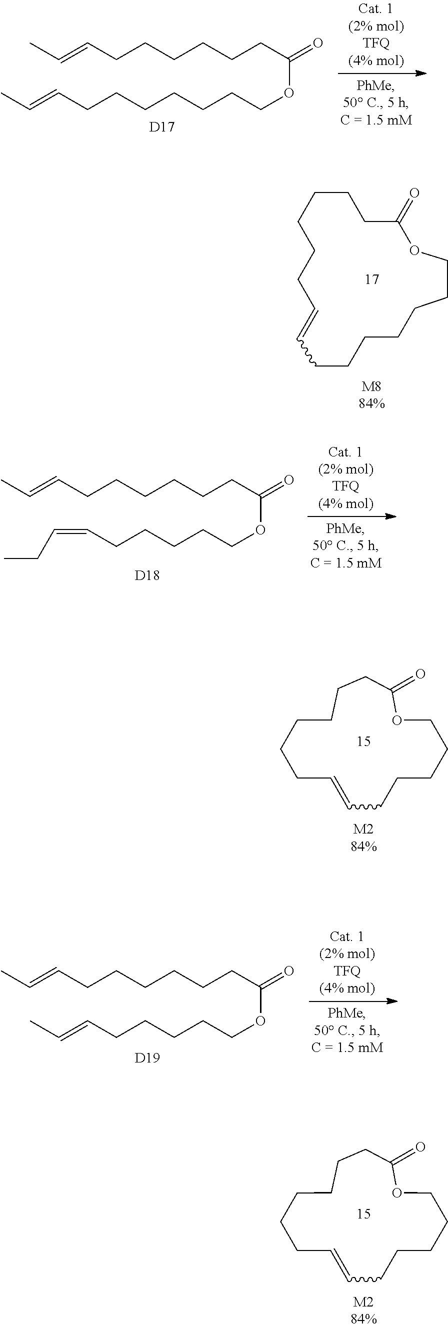

Cyclization of RCM dienes was performed in accordance with the general procedure described in example III, shortened RCM reaction conditions:
Solvent: anhydrous toluene [PhMe];
Reaction time: 5 hours;
Temperature: 50° C.;
Substrate concentration: 0.0015 mol/dm³ [C=1.5 mM];
Additives: tetrafluorobenzoquinone (4 mol %);
Catalyst [Ru]: Cat. 1 (2% mol)

TABLE 3
Cyclization of dienes with CH₃ substituents
at the C═C double bond in toluene.

| Item | Substrate | Product | Ring size | Efficacy [%] |
|---|---|---|---|---|
| 1. | D17 | M8 | 17 | 84 |
| 2. | D18 | M2 | 15 | 84 |
| 3. | D19 | M2 | 15 | 84 |

### Example VII

General Method for the RCM Reactions of Dienes with C═C Bonds Substituted with Short Alkyl Groups Only in the Acid Portion

Cyclization of RCM dienes was performed in accordance with the general procedure described in example III, shortened RCM reaction conditions:
Solvent: anhydrous toluene [PhMe];
Reaction time: 5 hours;
Temperature: 50° C.;
Substrate concentration: 0.0015 mol/dm³ [C=1.5 mM];
Additives: tetrafluorobenzoquinone (4 mol %);
Catalyst [Ru]: Cat. 1 (2% mol)

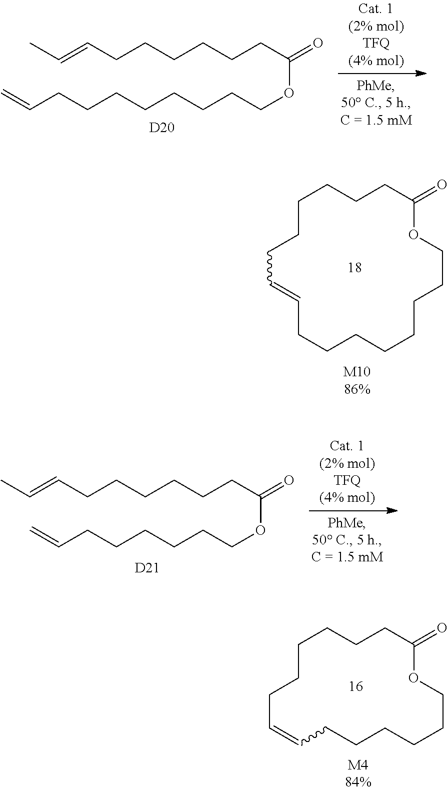

TABLE 4
Cyclization of dienes with short substituents at the C═C
double bond in the carboxy portion in toluene.

| Item | Substrate | Product | Ring size | Efficacy [%] |
|---|---|---|---|---|
| 1. | D21 | M4 | 16 | 84% |
| 2. | D20 | M10 | 18 | 86% |

### Example VIII

General Method for the RCM Reactions of Dienes with C═C Bonds Substituted with Short Alkyl Groups Only in the Alcohol Portion

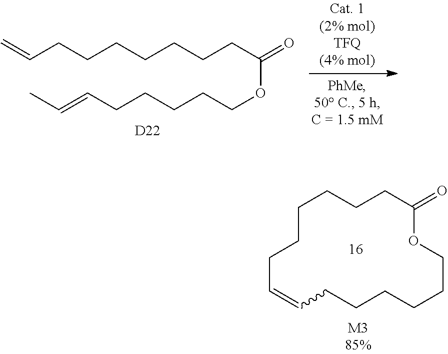

TABLE 5
Cyclization of diene D22 to macrocycle M3 in toluene.

| Item | Substrate | Product | Ring size | Efficacy [%] |
|---|---|---|---|---|
| 1. | D22 | M3 | 16 | 85 |

### Example IX

General Procedure for the RCM Macrocyclization Reaction in Ethyl Acetate, Atmospheric Pressure in the Presence of an Inert Gas.

Suitable ester (1 eq.) and tetrafluorobenzoquinone (4 mol %) were dissolved in anhydrous ethyl acetate (concentration of 12 to 100 mM) in a dried flask under atmosphere of inert gas. Precatalyst (Cat. 1, 2 mol % [20000 ppm] to 0.05 mol % [500 ppm]) was dissolved in dry ethyl acetate in a Schlenk flask (concentration of 1 mg/l mL) and added during the reaction at intervals of 15 minutes. The reaction was conducted at 77° C. for 5 to 24 hours. The progress of the reaction was monitored using thin layer chromatography (TLC). Once the reaction was over, the catalyst was deactivated by the addition of SnatchCat solution and stirred for 30 minutes. The solvent was evaporated, and the product was purified by column chromatography with 20% toluene/n-hexane (v/v).

The purpose of the example is to demonstrate the possibility of macrocyclization in ethyl acetate [Green Chem., 2014, 16, 1125-1130]. First, the results of the paper were reproduced with the same concentration (C=5 mM->for examples in toluene C=1.5 mM), and then model reactions were tested at a higher concentration range.

Reaction Conditions:
Solvent: anhydrous ethyl acetate [EtOAc];
Reaction time: 5 hours to 24 hours (detailed in Table 6);
Temperature: 77° C.;
Substrate concentration: 12 hours to 100 hours (detailed in Table 6);
Additives: tetrafluorobenzoquinone;
Catalyst [Ru]: Cat. 1;

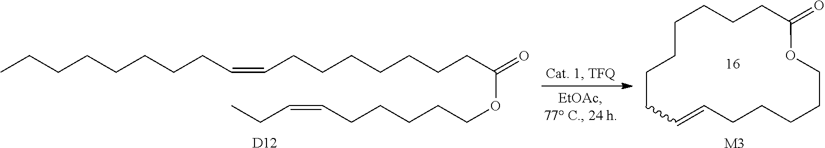

TABLE 6
RCM macrocyclization reactions of
cis-non-6-en-1-yl oleate (D12).

| | C_{substr} | Cat. 1 | Efficacy | E/Z isomer |
|---|---|---|---|---|
| Item | [mM] | [% mol] | [%] | ratio |
| 1. | 12 | 0.5 | 46 | 4.91 |
| 2. | 21 | 0.5 | 43 | 5.04 |
| 3. | 29 | 0.5 | 40 | 4.98 |
| 4. | 29 | 0.3 | 40 | 4.89 |
| 5. | 40 | 0.1 | 35 | 4.12 |
| 6. | 50 | 0.1 | 36 | 4.89 |
| 7. | 100 | 0.1 | 16 | 4.67 |
| 8. | 100 | 2 | 16 | ^{a} |
| 9.^{b} | 50 | 0.05 | 32 | 3.64 |
| | | [500 ppm] | | |

^{a}complex mixture of products
^{b}reaction conducted for 5 hours without no quinone addition

### Example X

List of Macrocyclic Products Obtained in the RCM Reaction

### a) (9-E, Z)-butadec-9-en-14-olide (M1)

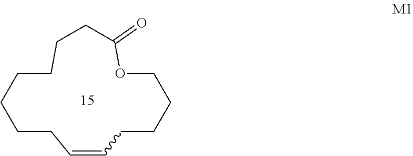 [SMILES: O=C1CCCCCCCC=CCCCCO1]

¹H NMR (400 MHz, CDCl₃): δ (ppm)=5.52-5.30 (m, 2H), 4.16-4.07 (m, 2H), 2.40-2.26 (m, 2H), 2.10-1.97 (m, 4H), 1.77-1.54 (m, 4H), 1.52-1.19 (m, 10H).

¹³C NMR (101 MHz, CDCl₃): δ (ppm)=174.24 (CO), 174.06 (CO), 131.58 (CH), 130.81 (CH), 130.79 (CH), 129.36 (CH), 63.98 (CH₂), 63.92 (CH₂), 34.69 (CH₂), 34.52 (CH₂), 31.86 (CH₂), 31.39 (CH₂), 28.62 (CH₂), 28.45 (CH₂), 28.12 (CH₂), 27.95 (CH₂), 27.94 (CH₂), 27.82 (CH₂), 27.64 (CH₂), 27.45 (CH₂), 27.36 (CH₂), 27.20 (CH₂), 27.10 (CH₂), 26.46 (CH₂), 25.62 (CH₂), 25.18 (CH₂), 25.00 (CH₂).

### b) (8-E, Z)-butadec-8-en-14-olide (M2)

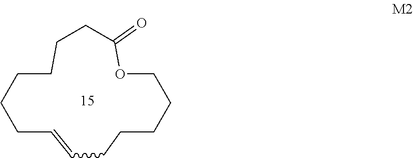 [SMILES: O=C1CCCCCCC=CCCCCCO1]

¹H NMR (400 MHz, CDCl₃): δ (ppm)=5.38-5.19 (m, 2H), 4.20-4.04 (m, 2H), 2.38-2.27 (m, 2H), 2.12-1.96 (m, 4H), 1.72-1.57 (m, 4H), 1.50-1.18 (m, 10).

¹³C NMR (100 MHz, CDCl₃): δ (ppm)=174.12 (CO), 131.40 (CH), 131.06 (CH), 63.44 (CH₂), 33.96 (CH₂), 31.98 (CH₂), 31.76 (CH₂), 28.55 (CH₂) 28.47 (CH₂) 28.35 (CH₂), 27.86 (CH₂), 27.13 (CH₂), 25.16 (CH₂), 24.95 (CH₂), 23.94 (CH₂).

### c) (9-E, Z)-pentadec-9-en-15-olide (M3)

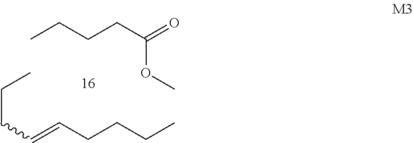

¹H NMR (400 MHz, CDCl₃): δ (ppm)=5.41-5.23 (m, 2H), 4.12-4.03 (m, 2H), 2.38-2.27 (m, 2H), 2.14-1.98 (m, 4H), 1.69-1.54 (m, 4H), 1.42-1.21 (m, 12H).

¹³C NMR (101 MHz, CDCl₃): δ (ppm)=174.10 (CO), 174.07 (CO), 131.43 (CH), 130.90 (CH), 130.38 (CH), 130.08 (CH), 64.67 (CH₂), 64.58 (CH₂), 34.88 (CH₂), 32.43 (CH₂), 31.61 (CH₂), 29.27 (CH₂), 29.17 (CH₂), 29.09 (CH₂), 28.83 (CH₂), 28.63 (CH₂), 28.41 (CH₂), 28.36 (CH₂), 28.24 (CH₂), 27.87 (CH₂), 27.84 (CH₂), 27.59 (CH₂), 27.39 (CH₂), 27.08 (CH₂), 26.53 (CH₂), 26.37 (CH₂), 25.65 (CH₂),25.10 (CH₂), 24.68 (CH₂).

### d) (8-E, Z)-pentadec-8-en-15-olide (M4)

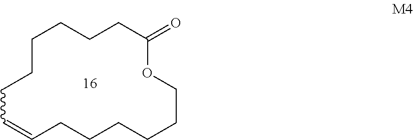 [SMILES: O=C1CCCCCCC=CCCCCCCO1]

¹H NMR (400 MHz, CDCl₃): δ (ppm)=5.49-5.22 (m, 2H), 4.19-4.04 (m, 2H), 2.36-2.28 (m, 2H), 2.09-1.96 (m, 4H), 1.73-1.56 (m, 4H), 1.49-1.21 (m, 12H).

¹³C NMR (100 MHz, CDCl₃): δ (ppm)=174.20 (CO), 130.93 (CH), 130.87 (CH), 130.24 (CH), 130.14 (CH), 64.45 (CH₂), 63.90 (CH₂), 34.97 (CH₂), 34.67 (CH₂), 31.84 (CH₂), 31.54 (CH₂), 29.55 (CH₂), 29.45 (CH₂), 28.99 (CH₂), 28.60 (CH₂), 28.45 (CH₂), 28.27 (CH₂), 28.19 (CH₂), 28.07 (CH₂), 27.82 (CH₂), 27.67 (CH₂), 27.38 (CH₂), 26.63 (CH₂), 26.47 (CH₂), 26.24 (CH₂), 26.03 (CH₂), 25.39 (CH₂), 25.24 (CH₂).

### e) (9-E, Z)-13-metylopentadec-9-en-15-olide (M5)

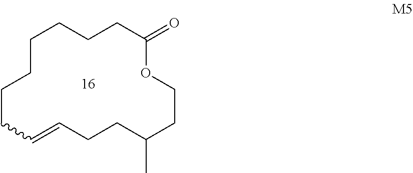 [SMILES: CC1CCC=CCCCCCCCC(=O)OCC1]

¹H NMR (400 MHz, CDCl₃): δ (ppm)=5.41-5.27 (m, 2H), 4.24-4.06 (m, 2H), 2.44-2.21 (m, 2H), 2.19-1.92 (m, 4H), 1.76-1.56 (m, 11H), 0.94-0.79 (m, 3H).

¹³C NMR (101 MHz, CDCl₃): δ (ppm)=174.05 (CO), 174.02 (CO), 131.04 (CH), 130.62 (CH), 130.53 (CH), 129.76 (CH), 62.20 (CH₂), 61.84 (CH₂), 36.59 (CH₂), 36.45 (CH₂), 36.41 (CH₂), 35.92 (CH₂), 35.20 (CH₂), 34.70 (CH₂), 31.26 (CH₂), 29.48 (CH₂), 29.01 (CH), 28.74 (CH₂), 28.36 (CH₂), 28.33 (CH₂), 28.14 (CH₂), 27.57 (CH₂), 27.54 (CH₂), 27.10 (CH₂), 26.79 (CH₂), 26.40 (CH₂), 26.37 (CH), 25.51 (CH₂), 25.08 (CH₂), 24.70 (CH₂), 19.46 (CH₃), 17.20 (CH₃).

### f) (9-E, Z:13-Z)-13-metylopentadec-9,13-dien-15-olide (M6)

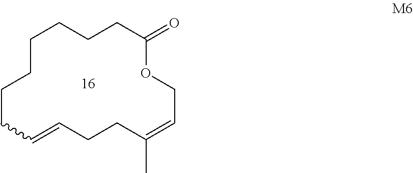 [SMILES: C/C1=C/COC(=O)CCCCCCCC=CCC1]

¹H NMR (400 MHz, CDCl₃): δ (ppm)=5.48-5.25 (m, 3H), 4.64-4.48 (m, 2H), 2.39-2.32 (m, 2H), 2.26-2.10 (m, 4H), 2.02-1.92 (m, 2H), 1.84-1.58 (m, 5H), 1.37-1.24 (m, 8H).

¹³C NMR (101 MHz, CDCl₃): δ (ppm)=174.38 (CO), 174.27 (CO), 145.72 (C), 141.41 (C), 131.14 (CH), 130.58 (CH), 129.40 (CH), 129.08 (CH), 120.02 (CH), 118.28 (CH), 60.94 (CH₂), 60.79 (CH₂), 34.27 (CH₂), 33.83 (CH₂), 32.27 (CH₂), 31.04 (CH₂), 30.98 (CH₂), 29.34 (CH₂), 27.65 (CH₂), 27.33 (CH₂), 27.30 (CH₂), 27.23 (CH₂), 27.21 (CH₂), 26.87 (CH₂), 26.65 (CH₂), 26.57 (CH₂), 26.13 (CH₂), 25.79 (CH₂), 24.92 (CH₂), 24.50 (CH₂), 24.26 (CH₃), 22.55 (CH₃).

### g) (9-E, Z;13-E)-13-metylopentadec-9, 13-dien-15-olide (M7)

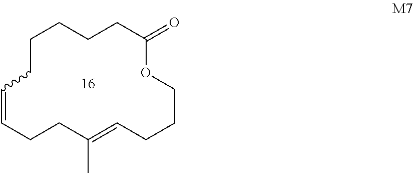 [SMILES: C/C1=C\CCCOC(=O)CCCCCC=CCC1]

¹H NMR (400 MHz, CDCl₃): δ (ppm)=5.47-5.20 (m, 3H), 4.56 (m, 2H), 2.39-2.29 (m, 2H), 2.24-2.08 (m, 4H), 2.05-1.91 (m, 2H), 1.75-1.53 (m, 5H), 1.36-1.25 (m, 8H).

¹³C NMR (101 MHz, CDCl₃): δ (ppm)=173.96 (CO), 173.90 (CO), 142.34 (C), 140.62 (C), 130.93 (CH), 130.13 (CH), 130.08 (CH), 129.52 (CH), 119.93 (CH), 118.67 (CH), 61.27 (CH₂), 61.18 (CH₂), 39.33 (CH₂), 39.00 (CH₂), 34.99 (CH₂), 34.37 (CH₂), 31.45 (CH₂), 29.05 (CH₂), 28.29 (CH₂), 27.91 (CH₂), 27.84 (CH₂), 27.73 (CH₂), 27.68 (CH₂), 27.58 (CH₂), 27.10 (CH₂), 27.05 (CH₂), 26.92 (CH₂), 26.29 (CH₂), 24.84 (CH₂), 24.30 (CH₂), 17.22 (CH₃), 15.53 (CH₃).

### h) (10-E, Z)-pentadec-10-en-15-olide (M12)

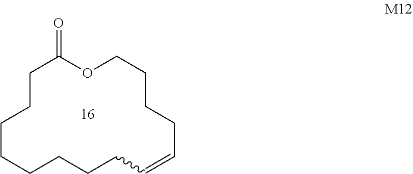 [SMILES: O=C1CCCCCCCCC=CCCCCO1]

¹H NMR (400 MHz, CDCl₃): δ (ppm)=5.45-5.24 (m, 2H), 4.17-4.09 (m, 2H), 2.38-2.28 (m, 2H), 2.12-1.96 (m, 4H), 1.70-1.54 (m, 4H), 1.47-1.14 (m, 12H).

¹³C NMR (101 MHz, CDCl₃): δ (ppm)=174.10 (CO), 131.97 (CH), 130.48 (CH), 130.26 (CH), 129.73 (CH), 64.26 (CH₂), 64.11 (CH₂), 34.91 (CH₂), 34.03 (CH₂), 32.18 (CH₂), 32.14 (CH₂), 29.30 (CH₂), 28.55 (CH₂), 28.48 (CH₂), 28.43 (CH₂), 28.36 (CH₂), 28.32 (CH₂), 28.15 (CH₂), 28.09 (CH₂), 27.77 (CH₂), 27.35 (CH₂), 27.34 (CH₂), 27.26 (CH₂), 26.75 (CH₂), 26.70 (CH₂), 26.62 (CH₂), 25.62 (CH₂), 25.40), 25.31 (CH₂).

### i) (8-E, Z)-hexadec-8-en-16-olide (M8)

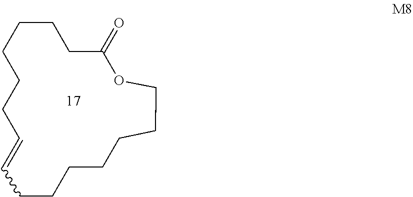 [SMILES: O=C1CCCCCCC=CCCCCCCCO1]

¹H NMR (400 MHz, CDCl₃): δ (ppm)=5.44-5.23 (m, 2H), 4.18-4.03 (m, 2H), 2.36-2.26 (m, 2H), 2.08-1.94 (m, 4H), 1.70-1.53 (m, 4H), 1.46-1.21 (m, 14H).

¹³C NMR (100 MHz, CDCl₃): δ (ppm)=174.28 (CO), 173.98 (CO), 131.44 (CH), 130.83 (CH), 130.33 (CH), 130.22 (CH), 64.47 (CH₂), 64.40 (CH₂), 34.72 (CH₂), 33.45 (CH₂), 31.49 (CH₂), 31.36 (CH₂), 29.03 (CH₂), 28.99 (CH₂), 28.86 (CH₂), 28.85 (CH₂), 28.81 (CH₂), 28.33 (CH₂), 27.91 (CH₂), 27.77 (CH₂), 27.58 (CH₂), 27.51 (CH₂), 26.89 (CH₂), 26.35 (CH₂), 26.32 (CH₂), 26.20 (CH₂), 25.92 (CH₂), 25.74 (CH₂), 25.16), 24.66 (CH₂).

### j) (9-E, Z)-hexadec-9-en-16-olide (M9)

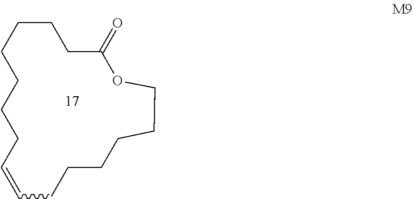 [SMILES: O=C1CCCCCCCC=CCCCCCCO1]

¹H NMR (400 MHz, CDCl₃): δ (ppm)=5.43-5.27 (m, 2H), 4.20-4.02 (m, 2H), 2.38-2.25 (m, 2H), 2.03 (m, 4H), 1.61 (m, 4H), 1.45-1.20 (m, 14H)

¹³C NMR (101 MHz, CDCl₃): δ (ppm)=174.21 (CO), 174.07 (CO), 131.32 (CH), 130.73 (CH), 130.47 (CH), 130.23 (CH), 64.51 (CH₂), 64.49 (CH₂), 35.05 (CH₂), 34.88 (CH₂), 31.92 (CH₂), 31.55 (CH₂), 29.77 (CH₂), 29.35 (CH₂), 29.02 (CH₂), 28.77 (CH₂), 28.75 (CH₂), 28.71 (CH₂), 28.63 (CH₂), 28.29 (CH₂), 28.10 (CH₂), 28.06 (CH₂), 27.99 (CH₂), 27.88 (CH₂), 27.08 (CH₂), 26.91 (CH₂), 26.63 (CH₂), 26.08 (CH₂), 25.94), 25.32 (CH₂), 25.17 (CH₂).

### k) (8-E, Z)-heptadec-8-en-17-olide (M10)

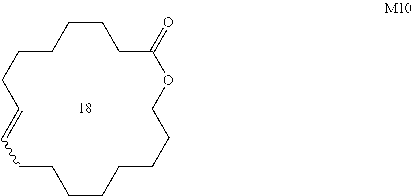 [SMILES: O=C1CCCCCCC=CCCCCCCCCO1]

¹H NMR (400 MHz, CDCl₃): δ (ppm)=5.53-5.16 (m, 2H), 4.15-4.05 (m, 2H), 2.31 (td, J=7.0, 5.3 Hz, 2H), 2.12-1.91 (m, 4H), 1.72-1.54 (m, 4H), 1.47-1.15 (m, 16H).

¹³C NMR (100 MHz, CDCl₃): δ (ppm)=173.55 (CO), 130.77 (CH), 130.74 (CH), 129.87 (CH), 129.86 (CH), 64.40 (CH₂), 63.99 (CH₂), 34.57 (CH₂), 34.48 (CH₂), 31.85 (CH₂), 31.86 (CH₂), 29.53 (CH₂), 29.14 (CH₂), 28.94 (CH₂), 28.96 (CH₂), 28.81 (CH₂), 28.78 (CH₂), 28.65 (CH₂), 28.60 (CH₂), 28.54 (CH₂), 28.35 (CH₂), 28.22 (CH₂), 27.86 (CH₂), 27.54 (CH₂), 27.32 (CH₂), 26.97 (CH₂), 26.46 (CH₂), 26.01 (CH₂), 25.80 (CH₂), 25.09 (CH₂), 25.00 (CH₂).

### I) (9-E, Z)-octadec-9-en-18-olide (M11)

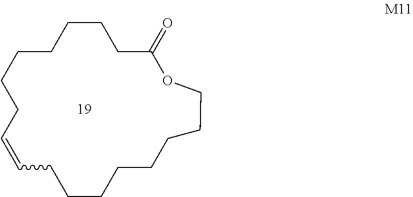 [SMILES: O=C1CCCCCCCC=CCCCCCCCCO1]

¹H NMR (400 MHz, CDCl₃): δ (ppm)=5.38-5.24 (m, 2H), 4.13-4.07 (m, 2H), 2.30 (t, J=6.9 Hz, 2H), 2.10-1.96 (m, 4H), 1.69-1.56 (m, 4H), 1.40-1.17 (m, 18H).

¹³C NMR (101 MHz, CDCl₃): δ (ppm)=174.34 (CO), 174.26 (CO), 130.96 (CH), 130.95 (CH), 130.43 (CH), 130.30 (CH), 64.74 (CH₂), 64.42 (CH₂), 35.16 (CH₂), 35.10 (CH₂), 32.30 (CH₂), 29.81 (CH₂), 29.70 (CH₂), 29.54 (CH₂), 29.47 (CH₂), 29.40 (CH₂), 29.31 (CH₂), 29.30 (CH₂), 29.27 (CH₂), 29.24 (CH₂), 29.11 (CH₂), 28.98 (CH₂), 28.97 (CH₂), 28.95 (CH₂), 28.25 (CH₂), 28.03 (CH₂), 27.92 (CH₂), 27.26 (CH₂), 26.47 (CH₂), 26.32 (CH₂), 25.48 (CH₂), 25.47 (CH₂).

### m) (10-E, Z)-oxacyclohexadec-10-en (M12)

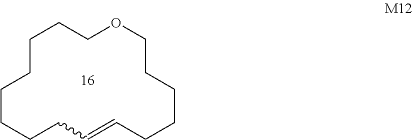 [SMILES: C1=CCCCCCOCCCCCCCC1]

¹H NMR (400 MHz, CDCl₃): δ (ppm)=5.38-5.25 (m, 2H), 4.46-4.36 (m, 4H), 2.15-1.95 (m, 4H), 1.60-1.48 (m, 4H), 1.46-1.36 (m, 6H), 1.35-1.24 (m, 8H).

¹³C NMR (101 MHz, CDCl₃): δ (ppm)=131.22 (CH), 130.93 (CH), 130.15 (CH), 130.03 (CH), 70.42 (CH₂O), 70.08 (CH₂O), 69.45 (CH₂O), 68.53 (CH₂O), 32.74 (CH₂), 32.18 (CH₂), 30.35 (CH₂), 30.14 (CH₂), 29.15 (CH₂), 29.04 (CH₂), 28.87 (CH₂), 28.72 (CH₂), 28.48 (CH₂), 28.27 (CH₂), 27.99 (CH₂), 27.72 (CH₂), 27.66 (CH₂), 27.63 (CH₂), 26.86 (CH₂), 26.81 (CH₂), 26.62 (CH₂), 26.22 (CH₂), 24.41), 23.99 (CH₂).

### Example XI

RCM Macrocyclization Reaction of Oct-7-En-1-Yl Oleate (D10) Under Conditions of Reduced Pressure and Elevated Temperature

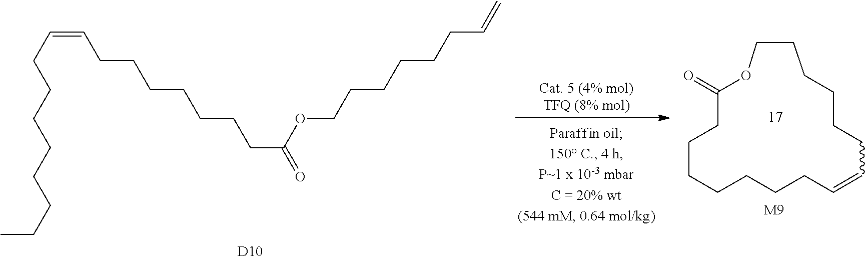

Oct-7-en-1-yl oleate (D10, 0.5 g; 1.28 mmol) in the presence of tetrafluorobenzoquinone (8 mol %) was subjected to RCM metathesis reaction with ruthenium catalyst Cat. 5 (4 mol %) in paraffin oil (CAS: 8012-95-1, Sigma-Aldrich) at reduced pressure (of the order of 1·10⁻³ mbar). The reaction was conducted for 4 hours at a temperature of 150° C. The resulting products, which distilled from the reaction mixture, were collected in a collector cooled with dry ice. The distillate was analysed by GCMS. The product was obtained with an effective yield of 12% and a purity of 12%, while the ratio of E and Z isomers of the compound M9 was 1.58.

Conclusion: conducting a RCM reaction with simultaneous distillation of the product is possible, the reaction requires optimisation of the conditions.

### Example XII

Metathesis Macrocyclization of Diene D12 Under Conditions of High Substrate Concentration and Reduced Pressure, Using Paraffin Oil as a Diluent

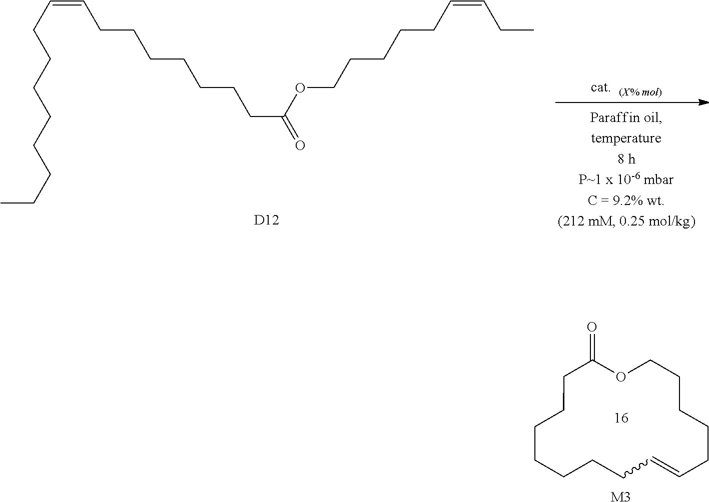

Paraffin oil (2 g, CAS: 8012-95-1, Sigma-Aldrich) and cis-non-6-en-1-yl oleate (D12, 203 mg; 0.5 mmol) were placed in a single-necked, 10 mL round flask equipped with a magnetic stirring element. A catalyst was then added (X % mol, X—a numerical value representing the catalyst feed, see Table 9). Immediately afterwards, the reaction flask was connected to a system with a collector connected to a diffusion pump. After evacuating the reaction system, the reaction flask was immersed in a heating bath at a predetermined temperature. Reaction mixture was stirred for the next 8 hours, while maintaining the lowest possible pressure (of the order of 1.10⁻⁶ mbar). During the reaction, products were collected in the collector attached to the reaction flask. After 8 hours, the connection of the reaction system to the diffusion flask was closed, the heating bath was removed, the stirring was stopped and the reaction system was opened so that air could access it. The crude product was eluted from the collector with hexane (20 mL), and then purified by chromatography using n-hexane and ethyl acetate. In the case of the reaction using 0.1 mol % or less of the catalyst, the ruthenium complex was introduced into the flask as a solution in dry and deoxygenated methylene chloride, after which the solvent was evaporated and the other ingredients were added.

Study of the metathetic process of cyclisation of cis-non-6-en-1-yl oleate (D12) was started by the optimization of the process temperature. The optimisation results are presented in Table 7. The experiments conducted showed that the minimum temperature at which the reaction is efficient is 110° C. The next step involved determining the catalytic activity of the available ruthenium complexes. Results of the catalytic activity of selected ruthenium complexes are summarized in Table 8. The following showed the greatest activity: Cat. 4, Cat. 5, Cat. 14 and Cat. 15. A series of experiments was then conducted to determine the minimum amount of ruthenium complex to ensure good reaction yield. The results of this part of studies are summarised in Table 9.

TABLE 7
Test results to determine the minimum temperature
of the process ensuring its good yield.

| | Cat. | Temperature | Efficacy M3 | Purity M3 | E/Z^{a} |
|---|---|---|---|---|---|
| Item | [0.5% mol] | [° C.] | [%] | [%] | isomer ratio |
| 1. | Cat. 4 | 95 | 10 | 83 | 2.28 |
| 2. | Cat. 4 | 100 | 10 | 94 | 4.19 |
| 3. | Cat. 4 | 105 | 9 | 93 | 4.18 |
| 4. | Cat. 4 | 110 | 98 | 97 | 3.08 |
| 5.^{b} | Cat. 4 | 120 | 36 | 95 | 2.54 |

^{a}Determined by gas chromatography (Purity = (signal intensity of E and Z isomers)/(signal intensity of all products)100%).
^{b}Reaction conducted at a pressure~ranging from 1 · 10⁻⁵ mbar to 5 · 10⁻⁴ mbar.

TABLE 8
Test results of the catalytic activity of selected ruthenium
complexes used in the amount of 0.5 mol %.

| | Cat. | Efficacy | Purity | E/Z^{a} |
|---|---|---|---|---|
| Item | [0.5% mol] | M3 (%) | M3 (%)^{a} | isomer ratio |
| 1. | Cat. 4 | 98 | 97 | 3.08 |
| 2. | Cat. 1 | 9 | 29 | 2.95 |
| 3. | Cat. 2 | 17 | 59 | 3.58 |
| 4. | Cat. 5 | 95 | 90 | 3.05 |
| 5. | Cat. 15 | 82 | 96 | 3.62 |
| 6. | Cat. 11 | 89 | 70 | 2.83 |
| 7. | Cat. 7 | 46 | 13 | 0.69 |
| 8. | Cat. 12 | 54 | 66 | 3.25 |
| 9. | Cat. 3 | 18 | 26 | 2.77 |
| 10. | Cat. 14 | 81 | 95 | 2.91 |
| 11. | Cat. 13 | 22 | 78 | 4.08 |

^{a}Determined by gas chromatography (Purity = (signal intensity of E and Z isomers)/(signal intensity of all products)100%).

TABLE 9
Results of catalytic tests of the most active ruthenium complexes
to determine the lowest effective amount of the catalyst.

| | Catalyst | Catalyst quantity | Efficacy | Purity | E/Z^{a} |
|---|---|---|---|---|---|
| Item | [Ru] | [% mol] | M3 [%] | M3 [%] | isomer ratio |
| 1. | Cat. 4 | 0.5 | 98 | 97 | 3.08 |
| 2. | Cat. 4 | 0.5 | 93 | 98 | 2.93 |
| 3. | Cat. 4 | 0.1 | 3 | 98 | 2.56 |
| 4. | Cat. 4 | 0.1 | 0 | — | — |
| 5. | Cat. 15 | 0.5 | 82 | 96 | 3.62 |
| 6. | Cat. 15 | 0.1 | 0 | — | — |
| 7. | Cat. 14 | 0.5 | 81 | 95 | 2.91 |
| 8. | Cat. 14 | 0.1 | 12 | 99 | 2.40 |
| 9. | Cat. 5 | 0.5 | 95 | 90 | 3.05 |
| 10. | Cat. 5 | 0.1 | 39 | 95 | 2.95 |
| 11.^{b} | Cat. 5 | 0.1 | 66 | 87 | 2.67 |
| 12.^{b} | Cat. 5 | 0.05 | 38 | 95 | 2.60 |
| 13.^{b} | Cat. 5 | 0.2 | 81 | 93 | 2.92 |

^{a}Determined by gas chromatography (Purity = (signal intensity of E and Z isomers)/(signal intensity of all products)100%).
^{b}The substrate and paraffin were filtered through Al₂O₃.

### Example XIII

Metathesis Macrocyclization of D12 Diene Under Reduced Pressure in Various High-Boiling Diluents.

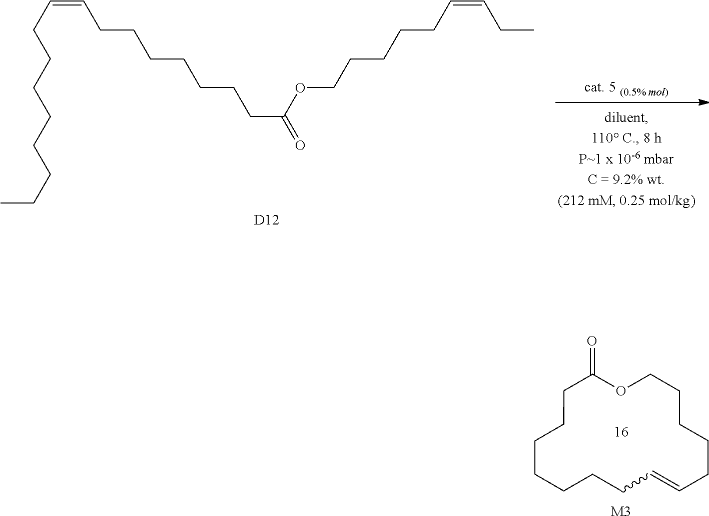

The same reaction conditions were used as in example X using various high-boiling diluents ((i) paraffin oil [CAS: 8012-95-1; manufacturer: Sigma-Aldrich]; (ii) paraffin wax/solid paraffin [CAS: 8002-74-2; manufacturer: Aldrich]; (iii) polyethylene [CAS: 9002-88-4; manufacturer Aldrich]; (iv) ionic liquid [1-butyl-2,3-dimethylimidazolium hexafluorophosphate; CAS: 227617-70-1; manufacturer: Aldrich]). The cumulative results are presented in Table 10 below.

TABLE 10
Results of experiments for selecting the optimal diluent.

| | | Efficacy | Purity | E/Z^{a} |
|---|---|---|---|---|
| Item | Diluent | M3 (%) | M3 (%)^{a} | isomer ratio |
| 1. | Paraffin oil | 95 | 87 | 3.32 |
| 2. | Paraffin wax | 91 | 86 | 3.08 |
| 3. | Polyethylene | 26 | 92 | 3.47 |
| 4. | Ionic liquid^{b} | 33 | 77 | 3.44 |

Conditions: cis-non-6-en-1-yl oleate (D12, 0.203 g; 1.28 mmol); Cat. 5 (0.5 mol %); diluent (2 g); temperature = 110° C.; pressure of the order of 1 · 10⁻⁶ mbar; reaction time = 8 hours.
^{a}Determined by GC (Purity = (signal intensity of E and Z isomers)/(signal intensity of all products)100%).
^{b}1-butyl-2,3-dinnethylimidazoliunn hexafluorophosphate;

### Example XIV

Metathesis Macrocyclization Reactions Yielding M3 Using Dienes with C═C Bonds with Various Substituents as Substrates.

General Procedure of Metathesis Cyclization:

Paraffin oil (2 g, CAS: 8012-95-1, Sigma-Aldrich; filtered through a neutral Al₂O₃ gel) and a substrate for ring metathesis reaction (0.5 mmol; molar concentration in the reaction mixture=0.25 mol/kg) were placed in a single-necked, 10 mL round flask equipped with a magnetic stirring element. Next, a catalyst Cat. 5 was added 5 (2.5 μmol, 0.5 mol %). Immediately afterwards, the reaction flask was connected to a system with a collector connected to a diffusion pump. After evacuating the reaction system, the reaction flask was immersed in a heating bath at a predetermined temperature. This was stirred for the next 8 hours, while maintaining the lowest possible pressure (lowest value obtained: 4·10⁻⁶ mbar). During the reaction, products were collected in the collector attached to the reaction flask. After 8 hours, the connection of the reaction system to the diffusion pump was closed, the heating bath was removed, the stirring was stopped and the reaction system was opened so that air could access it. The crude product was eluted from the collector with hexane (20 mL), and then the solvents were evaporated under reduced pressure and purified by chromatography using n-hexane and ethyl acetate.

TABLE 19
Metathesis cyclization reactions illustrating the effect of substituting C═C bonds
in a substrate molecule

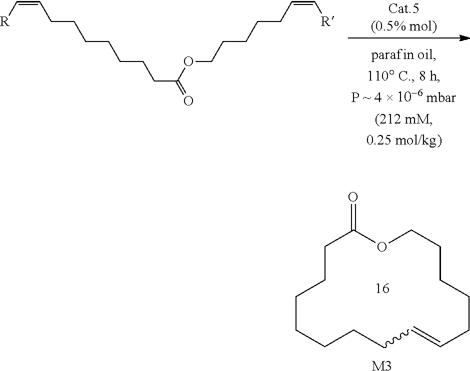

| Item | Diene | Substituent R | Substituent R’ | Efficacy (%) | Purity (%)^{a} | E/Z^{a} isomer ratio |
|---|---|---|---|---|---|---|
| 1. | D12 | n-C₈H₁₇ | C₂H₅ | 100 | 82 | 2.93 |
| 2. | D4 | H | C₂H₅ | 96 | 90 | 3.34 |
| 3. | D24 | n-C₈H₁₇ | H | 64 | 92 | 3.10 |
| 4. | D25 | H | H | 87 | 90 | 3.28 |

^{a}Determined by GC (Purity = (signal intensity of E and Z isomers)/(signal intensity of all products) 100%).

Conclusion: The metathesis macrocyclization reaction is effective both for substrates with terminal C═C bonds and for substrates with C═C bonds substituted with alkyl groups.

### Example XV

Demonstration of the RCM Reaction Course of a Diolefin Substrate Through the Stage of Cross Metathesis Products Between Molecules of Said Compound (Oligomers/Polymers)

Description of Experiment 1:

Paraffin oil (2 g, CAS: 8012-95-1, Sigma-Aldrich; filtered through a neutral Al₂O₃ gel) and D25 compound (133.0 mg; 0.5 mmol, C=6.2% w/w, molar concentration in the reaction mixture=0.25 mol/kg) were placed in a single-necked, 10 mL round flask equipped with a magnetic stirring element. Next, a catalyst Cat. 5 was added (1.8 g; 2.5 μmol, 0.5 mol %). Immediately afterwards, the reaction flask was connected to a system with a collector connected to a diffusion pump. After evacuating the reaction system, the reaction flask was immersed in a heating bath at a temperature of 110° C. After 15 minutes, the valve connecting the reaction system with the diffusion pump was closed and the system was opened in the air. A sample was taken from the reaction mixture, which upon analysis showed complete conversion of the D25 substrate and the presence of a small amount of oligomers and polymeric substances formed by acyclic metathesis of the D25 diene with a small amount of the M3 product. The resulting oligomers and polymeric substances were isolated using column chromatography, and then subjected to MALDI-TOF MS analysis, which showed the presence of compounds with a mass of more than 1000 Da.

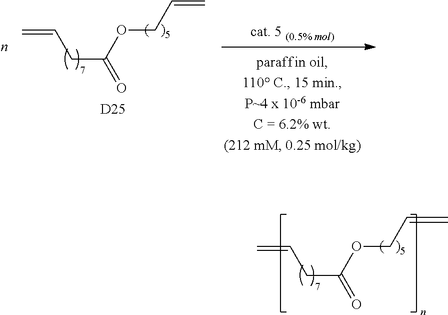

### Diagram 1. An Experiment Illustrating the Early Stage of the RCM Reaction Yielding a Macrocyclic Product

See FIG. 7a for the illustration of the result of MALDI-TOF MS spectrometry analysis.

See FIG. 8a for the illustration of the result of thin layer chromatography analysis (eluent: n-hexane/ethyl acetate=100/5; v/v). Depicted from the left: D25 substrate standard, reaction mixture, M3 product standard, all co-deposited substances. The analysis illustrates the complete conversion of the substrate within a short time from the start of the reaction, with the main products being oligomers and polymeric substances.

Description of Experiment 2:

Paraffin oil (2 g, CAS: 8012-95-1, Sigma-Aldrich; filtered through a neutral Al₂O₃ gel) and compound D12 (203.0 mg; 0.5 mmol; C=9.2% w/w, molar concentration in the reaction mixture=0.25 mol/kg) were placed in a single-necked, 10 mL round flask equipped with a magnetic stirring element. Next, a catalyst Cat. 5 was added (1.8 mg; 2.5 μmol; 0.5 mol %). Immediately afterwards, the reaction flask was connected to a system with a collector connected to a diffusion pump. After evacuating the reaction system, the reaction flask was immersed in a heating bath at a temperature of 110° C. After 20 minutes, the valve connecting the reaction system with the diffusion pump was closed and the system was opened in the air. A sample was taken from the reaction mixture, which upon analysis showed complete conversion of the D12 substrate and the presence of a small amount of oligomers and polymeric substances formed by acyclic metathesis of the D12 diene. The resulting oligomers and polymeric substances were isolated using column chromatography, and then subjected to MALDI-TOF MS analysis, which showed the presence of compounds with a mass of more than 1000 Da.

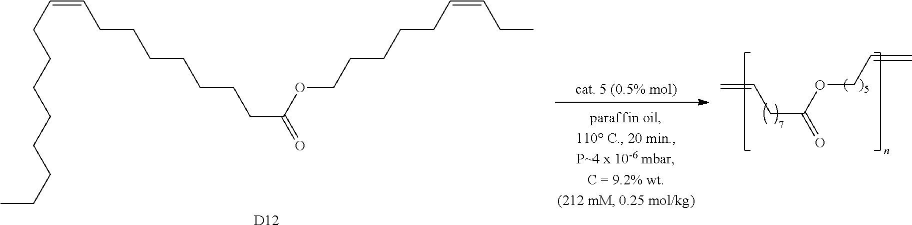

### Diagram 2. An Experiment Illustrating the Early Stage of the RCM Reaction Yielding a Macrocyclic Product

See FIG. 7b for the illustration of the result of MALDI-TOF MS spectrometry analysis.

See FIG. 8b for the illustration of the result of thin layer chromatography analysis (eluent: n-hexane/ethyl acetate=100/5; v/v). Depicted from the left: D12 substrate standard, reaction mixture, M3 product standard, all co-deposited substances. The analysis illustrates the complete conversion of the substrate within a short time from the start of the reaction, with the main products being oligomers and polymeric substances.

Conclusion: While example XIV illustrates that D12 and D25 are efficiently converted into macrocyclic M3 after 8 hours, example XIV shows that these substrates under the conditions used (the same catalyst in the same amount, the same diluent and substrate concentration, the same temperature and pressure) are fully converted to form oligomers and polymeric substances within a short time from the start of the reaction. This means that oligomers and polymeric substances are the transitional stage of the course yielding the macrocyclic product.

### Example XVI

Demonstration of the Applicability of the Method in Relation to Substrates with Various Structure

The experiments presented below were conducted according to the procedure presented in Example XIV.

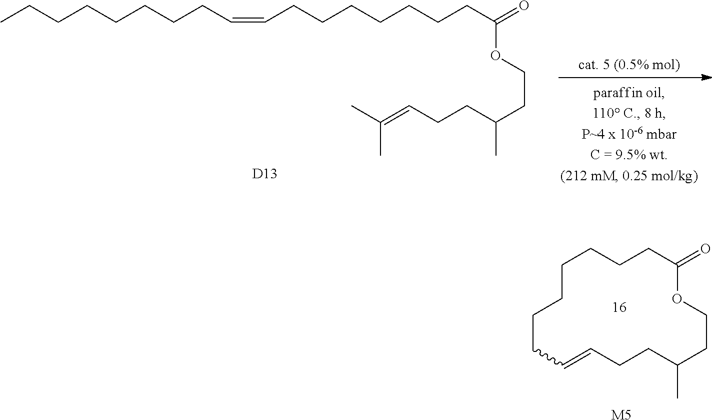 [SMILES: CCCCCCCC/C=C\CCCCCCCC(=O)OCCC(C)CCC=C(C)C ; "D13" ; CC1CCC=CCCCCCCCC(=O)OCC1 ; "M5"]

### Diagram 3. Metathesis cyclization of 3,7-dimethyl-oct-6-en-1-yl oleate (yield=40%, purity=87%, E/Z=4.19)

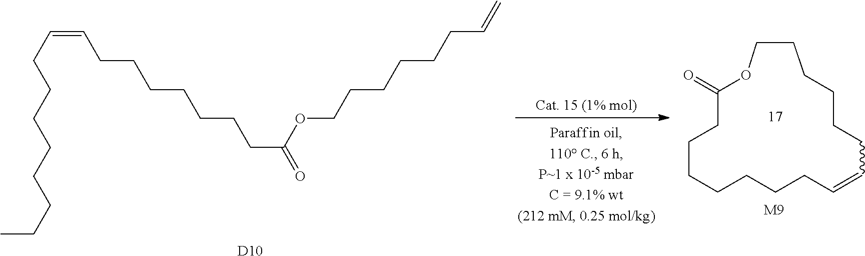

### Diagram 4. Metathesis cyclization of oct-7-en-1-yl oleate (yield=43%, purity=80%, E/Z=2.10)

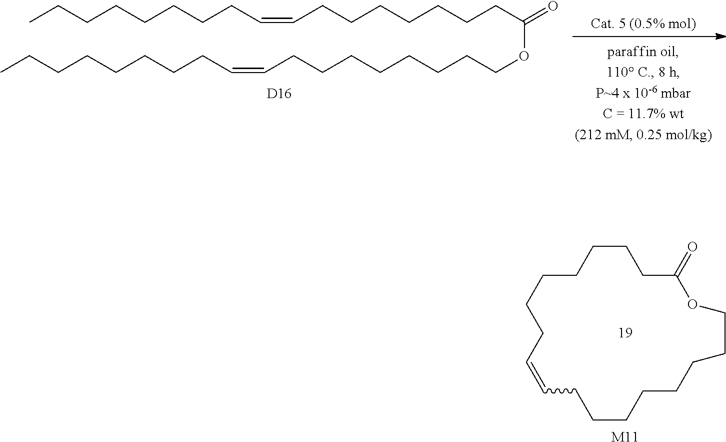

### Diagram 5. Metathesis cyclization of oleyl oleate (yield=23%, purity=93%, E/Z=3.76)

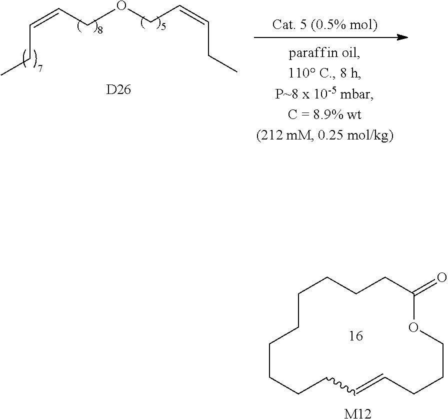

### Schemat 6. Metathesis cyclization of (9-Z)-1-(((6-Z)-non-6-en-1-yl)oxy)octadeca-9-ene (yield=28%, purity=90%, E/Z=3.11)

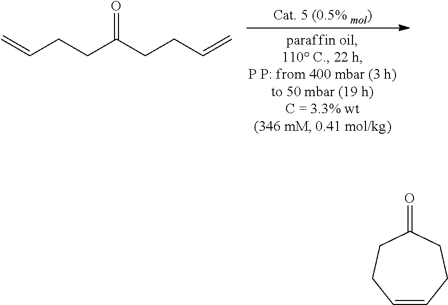

### Diagram 7. Metathesis cyclization of nona-1, 8-dien-5-on (yield=64%)

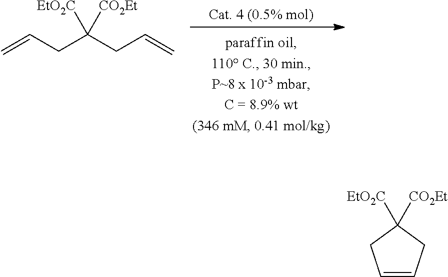 [SMILES: C=CCC(CC=C)(C(=O)OCC)C(=O)OCC ; CCOC(=O)C1(C(=O)OCC)CC=CC1]

### Diagram 8. Metathesis cyclization of diethyl diallylmalonate (yield=85%)

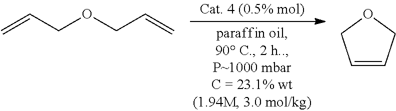

### Diagram 9. Metathesis cyclization of diallyl ether (yield=24%)

Conclusions: the examples above confirm that the method for the preparation of unsaturated cyclic compounds of the invention is useful for using it for substrates with various structure and ring sizes.

### Example XVII (Complementing Example XVI)

Demonstration of the Applicability of the Method in Relation to Substrates with Various Structure

General Procedure of Metathesis Cyclization:

Paraffin oil (2 g, CAS: 8012-95-1, Sigma-Aldrich; filtered through a neutral Al₂O₃ gel) and a substrate for the ring metathesis reaction (0.5 mmol, molar concentration in the reaction mixture=0.25 mol/kg) were placed in a single-necked, 10 mL round flask equipped with a magnetic stirring element. Next, catalyst Cat. 5 was added. Immediately afterwards, the reaction flask was connected to a system with a collector connected to a diffusion pump. After evacuating the reaction system, the reaction flask was immersed in a heating bath at a temperature determined as 110° C. This was stirred for the next 8 hours, while maintaining the lowest possible pressure. The vacuum level was read from the vacuum meter connected directly to the diffusion pump (the vacuum was of the order of 10⁻⁶). During the reaction, products were collected in the collector attached to the reaction flask. After 8 hours, the connection of the reaction system to the diffusion pump was closed, the heating bath was removed, the stirring was stopped and the reaction system was opened so that air could access it. The crude product was eluted from the collector with hexane (20 mL), and then the solvents were evaporated under reduced pressure and purified by chromatography using n-hexane and ethyl acetate.

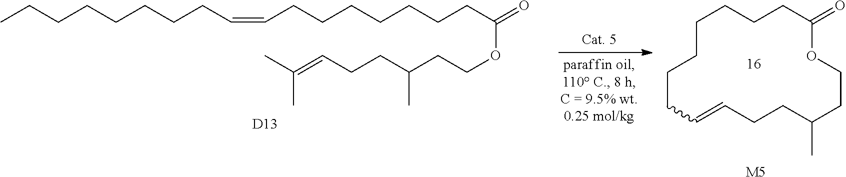 [SMILES: CCCCCCCC/C=C\CCCCCCCC(=O)OCCC(C)CCC=C(C)C ; "D13" ; CC1CCC=CCCCCCCCC(=O)OCC1 ; "M5"]

### Diagram 10. Metathesis cyclization of 3,7-dimethyl-oct-6-en-1-yl oleate

For 1 mol % Cat. 5: yield=51%, purity=91%, E/Z proportion=4.3

For 2 mol % Cat. 5: yield=56%, purity=92%, E/Z proportion=4.3

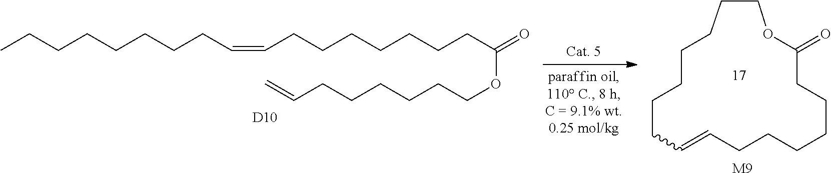

### Diagram 11. Metathesis cyclization of 7-oct-1-enyl oleate

For 1 mol % Cat. 5: yield=75%, purity=57%, E/Z proportion=2.1

For 2 mol % Cat. 5: yield=79%, purity=55%, E/Z proportion=2.2

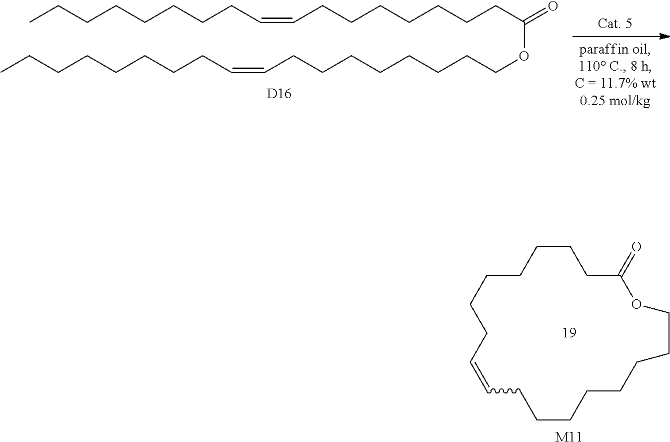

### Diagram 12. Metathesis cyclization of oleyl oleate

For 2 mol % Cat. 5: yield=55%, purity=76%, E/Z proportion=3.3

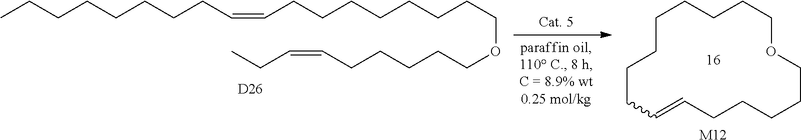

### Diagram 13. Metathesis cyclization of (9Z)-1-(((6Z)-non-6-en-1-ylo)oxy)octadec-9-ene

For 1% mol Cat. 5: yield=93%, purity=97%, E/Z proportion=4.9

### Example XVIII (Complementing Example XIII)

Metathesis Macrocyclization of D12 Diene Under Reduced Pressure in Various High-Boiling Diluents.

General Procedure of Metathesis Cyclization Using PAO as Diluent

PAO (2 g, filtered through a neutral Al₂O₃ gel) and cis-non-6-en-1-yl oleate (0.5 mmol, molar concentration in the reaction mixture=0.25 mol/kg) were placed in a single-necked, 10-mL round flask equipped with a magnetic stirring element. Next, catalyst Cat. 5 was added. Immediately afterwards, the reaction flask was connected to a system with a collector connected to a diffusion pump. After evacuating the reaction system, the reaction flask was immersed in a heating bath at a predetermined temperature. This was stirred for the next 8 hours, while maintaining the lowest possible pressure. The vacuum level was read from the vacuum meter connected directly to the diffusion pump (the vacuum was of the order of 10⁻⁶). During the reaction, products were collected in the collector attached to the reaction flask. After 8 hours, the connection of the reaction system to the diffusion pump was closed, the heating bath was removed, the stirring was stopped and the reaction system was opened so that air could access it. The crude product was eluted from the collector with hexane (20 mL), and then the solvents were evaporated under reduced pressure and purified by chromatography using n-hexane and ethyl acetate.

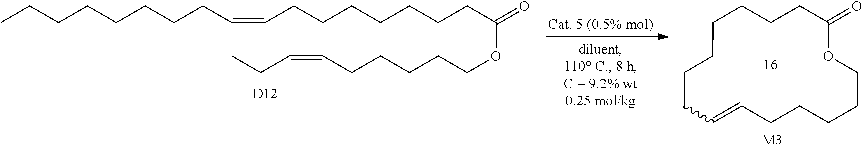

TABLE 12
Results of experiments for selecting the
optimal diluent (complementing table 10).

| | | Yield | Purity | Ratio of |
|---|---|---|---|---|
| Item | Diluent | M3 (%) | M3 (%)^{a} | E/Z^{a} isomers |
| 1 | PAO 4 | 97 | 96 | 3.4 |
| 2 | PAO 6 | 99 | 95 | 3.4 |

Conditions: cis-non-6-en-1-yl oleate (D12, 0.203 g; 1.28 mmol); Cat. 5 (0.5 mol %); diluent (2 g);
temperature = 110° C.; pressure measured at the punnp of the order of x*10⁻⁶ mbar; reaction time = 8 hours.
^{a}Determined by GC (Purity = (signal intensity of E and Z isomers)/(signal intensity of all products)100%).

Information from specification sheets of PAO4 and PAO6 used:

PAO4, SpectraSyn™ 4 Polyalphaolefin, ExxonMobil Chemical

Composition (CAS-No./EINECS-No.: Various): Branched Alkanes
Kinematic Viscosity, cSt @ 212° F., 100° C. 4.1
Kinematic Viscosity, cSt @ 104° F., 40° C. 19.0
Kinematic Viscosity, cSt @ −40° F., −40° C. 2.900
Viscosity Index 126
Pour Point, ° F., ° C. −87 (−66)
Flash Point (COC), ° F., ° C. 428 (220)
Fire Point (COC), ° F., ° C. 493 (256)
Volatility, Noack, wt % 14.0
Specific Gravity, 60°/60° F., 15.6°/15.6° C. 0.820
Total Acid Number <0.05
Odor No Foreign Odor
Appearance Clear and Bright
Color, Pt—Co <0.5

PAO6, Synfluid® PAO 6 cSt, Chevron Phillips Chemical Company LP

Composition (CAS-No. / EINECS-No.: 68037-01-4):
1-Decene Homopolymer Hydrogenated 100%
Kinematic Viscosity, cSt @ 212° F., 100° C. 5.9
Kinematic Viscosity, cSt @ 104° F., 40° C. 30.5
Kinematic Viscosity, cSt @ −40° F., −40° C. 7.712
Viscosity Index 137
Pour Point, ° F., ° C. −78 (−61)
Flash Point (COC), ° F., ° C. 473 (245)
Fire Point (COC), ° F., ° C. 529 (276)
Volatility, Noack, wt % 6.6
Specific Gravity, 60°/60° F., 15.6°/15.6° C. 0.8278
Density, lb/gal 6.893
Total Acid Number <0.03
Bromine Index <200
Odor No Foreign Odor
Appearance Clear and Bright
Color, Pt—Co 0

### Example XIX (Complementing Example XII to Table 8)

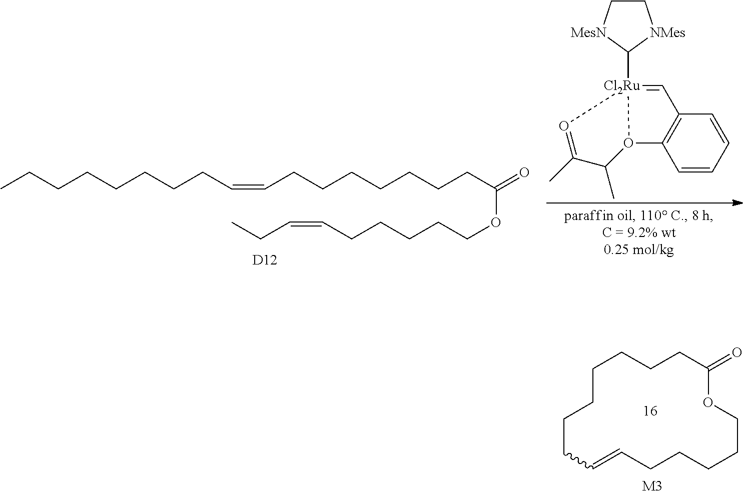

### Diagram 14. Metathesis cyclization of cis-non-6-en-1-yl oleate using Umicore M51™ complex

TABLE 13
Metathesis cyclization of cis-non-6-en-1-yl oleate using
Umicore M51 ™ complex (FIG. 9).

| | Catalyst quantity | Yield | Purity | Ratio of |
|---|---|---|---|---|
| Item | [% mol] | M3 (%) | M3 (%)^{a} | E/Z^{a} isomers |
| 1 | 1 | 51 | 23 | 3.4 |
| 2 | 0.5 | 33 | 29 | 3.6 |

### Example XX

Metathesis Macrocyclization of Diene D12 Under Conditions of High Substrate Concentration and Reduced Pressure (Reactions Using Rotary Oil Pump)

General Procedure of Metathesis Cyclization Using Rotary Oil Pump

Paraffin oil (2 g, CAS: 8012-95-1, Sigma-Aldrich; filtered through a neutral Al₂O₃ gel) or PAO6 (filtered through a neutral Al₂O₃ gel) and a substrate for the ring metathesis reaction (concentration per weight and molar concentration are given for particular reactions in respective diagrams) were placed in a single-necked, 10 mL round flask equipped with a magnetic stirring element. Next, a catalyst Cat. 5 or Cat. 4 was added. Immediately afterwards, the reaction flask was connected to a system with a collector connected to an oil pump. After evacuating the reaction system, the reaction flask was immersed in a heating bath at a predetermined temperature. During the reaction, products were collected in the collector attached to the reaction flask. The nominal pressure of the pump (read from the vacuum gauge) was 1*10⁻³, real pressure in the apparatus (read on the MacLeod gauge) was 1*10⁻².

After 6 hours, the connection of the reaction system to the oil pump was closed, the heating bath was removed, the stirring was stopped and the system was filled with inert gas (argon). The next day, a fresh portion of the catalyst was added and the reaction was continued for another 6 hours. Following an appropriate number of cycles, the crude product was eluted from the collector with n-hexane (20 mL), and then the solvents were evaporated under reduced pressure and purified by chromatography using n-hexane and ethyl acetate.

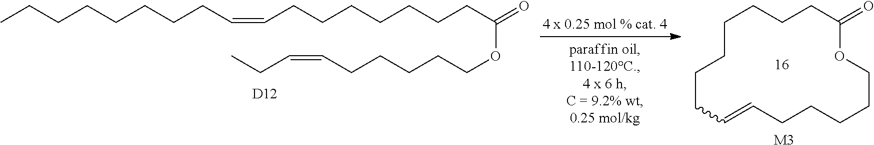

### Diagram 15. Reaction of cis-non-6-en-1-yl oleate conducted 4×6 h

Yield=92%, purity=93%, E/Z ratio=3.7

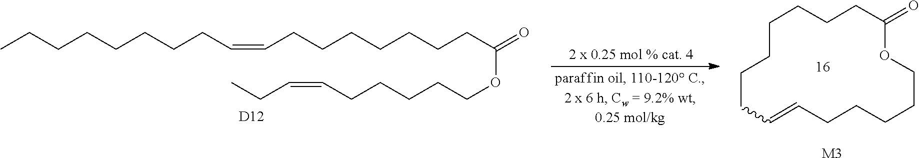

### Diagram 16. Reaction of cis-non-6-en-1-yl oleate conducted 2×6 h

Yield=92%, purity=96%, E/Z ratio=3.5

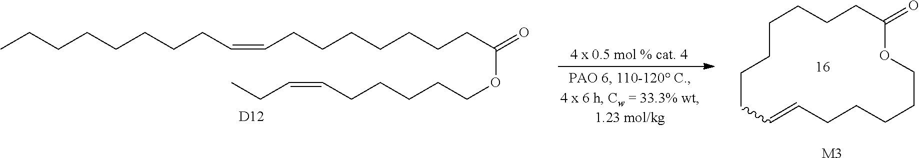

### Diagram 17. Reaction of cis-non-6-en-1-yl oleate conducted 2×6 h in 3 g scale

Yield=78%, purity=87%, E/Z ratio=3.5

### Example XXI

Metathesis Cyclization of Civetone

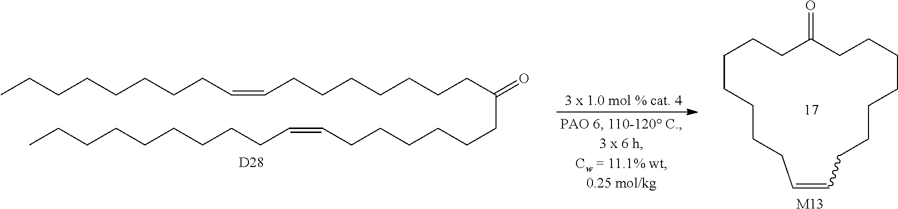

### Diagram 18. Macrocyclization of Civetone

Yield=69%, purity=86%, E/Z ratio=3.0

### Example XXII

### Macrocyclization of cyclohept-4-en-1-one

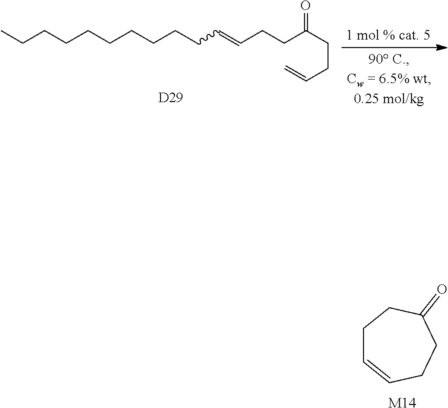

### Diagram 19. Macrocyclization of cyclohept-4-en-1-one

TABLE 14
Results for macrocyclization of cyclohept-
4-en-1-one for various procedures.

| | Time [h] | diluent | Yield |
|---|---|---|---|
| 1 | 6 | Paraffin oil | 45% |
| 2 | 24 | Paraffin oil | 59% |
| 3 | 24 | Paraffin oil | 81% |
| 4 | 24 | PAO6 | 79% |

Procedure 1.

Paraffin oil (2.72 g, CAS: 8012-95-1, Sigma-Aldrich; filtered through a neutral Al₂O₃ gel) and a substrate for the ring metathesis reaction D29 (0.68 mmol, molar concentration in the reaction mixture=0.25 mol/kg) were placed in a single-necked, 10 mL round flask equipped with a magnetic stirring element. Next, a catalyst Cat. 5 was added (0.01 mmol, 1.0 mol %). Immediately afterwards, the reaction flask was connected to a system with a collector connected to a membrane vacuum pump membrane vacuum pump. After evacuating the reaction system, the reaction flask was immersed in a heating bath at a temperature determined as 90° C. This was stirred for the next 6 hours, while maintaining the lowest possible pressure of 7-8 mbar indicated by the vacuum meter of the membrane vacuum pump. During the reaction, products were collected in the collector attached to the reaction flask. After 6 hours, the connection of the reaction system to the membrane pump was closed, the heating bath was removed, the stirring was stopped and the reaction system was opened so that air could access it. The crude product was eluted from the collector with diethyl ether (20 mL), then evaporated on a rotary evaporator at a pressure of 300 mbar at room temperature. 34 mg of the product was obtained with a purity of 99%, equivalent to a 45% yield. (Table 2, example 1)

Procedure No. 2.

Paraffin oil (4 g, CAS: 8012-95-1, Sigma-Aldrich; filtered through a neutral Al₂O₃ gel) and a substrate for the ring metathesis reaction D29 (1.0 mmol, molar concentration in the reaction mixture=0.25 mol/kg) were placed in a single-necked, 10 mL round flask equipped with a magnetic stirring element. In parallel, a stopcock (ø14) was prepared connected to a cold trap. Next, a catalyst Cat. 5 was added (0.01 mol, 1.0 mol %). Immediately afterwards, the reaction flask was connected to a system with a collector connected to a membrane vacuum pump. After evacuating the reaction system, the cold trap was immersed in liquid nitrogen, and the reaction flask—in a heating bath at a temperature determined as 90° C. This was stirred for the next 24 hours, while maintaining the lowest possible pressure of 7-8 mbar indicated by the vacuum meter of the membrane vacuum pump. During the reaction, products were collected in the collector attached to the reaction flask. After 24 hours, the connection of the reaction system to the membrane vacuum pump was closed, the heating bath was removed, the stirring was stopped and the reaction system was opened so that air could access it. The crude product was eluted from the cold trap with diethyl ether (20 mL), then evaporated on a rotary evaporator at a pressure of 300 mbar at room temperature. 80 mg of the product was obtained with a purity of 81%, equivalent to a 59% yield. (Table 2, example 2)

Procedure No. 3.

Paraffin oil (4 g, CAS: 8012-95-1, Sigma-Aldrich; filtered through a neutral Al2O3 gel) and a substrate for the ring metathesis reaction D29 (0.5 mmol, molar concentration in the reaction mixture=0.25 mol/kg) were placed in a single-necked, 10 mL round flask equipped with a magnetic stirring element. In parallel, a stopcock (ø14) was prepared connected to a cold trap mounted backwards. Next, a catalyst Cat. 5 was added (0.01 mol, 1.0 mol %). Immediately afterwards, the reaction flask was connected to a system with a collector connected to a membrane vacuum pump. After evacuating the reaction system, the cold trap was immersed in liquid nitrogen, and the reaction flask—in a heating bath at a temperature determined as 90° C. This was stirred for the next 24 hours, while maintaining the lowest possible pressure of 7-8 mbar indicated by the vacuum meter of the membrane vacuum pump. During the reaction, products were collected in the collector attached to the reaction flask. After 24 hours, the connection of the reaction system to the membrane vacuum pump was closed, the heating bath was removed, the stirring was stopped and the reaction system was opened so that air could access it. The crude product was eluted from the cold trap with diethyl ether (20 mL), then evaporated on a rotary evaporator at a pressure of 300 mbar at room temperature. 99 mg of the product was obtained with a purity of 90%, equivalent to a 81% yield. (Table 2, example 3)

Procedure No. 4.

A polyalphaolefin—PAO6 (4 g, filtered through a neutral Al₂O₃ gel) and a substrate for ring metathesis reaction D29 (1.0 mmol, molar concentration in the reaction mixture=0.25 mol/kg) were placed in a single-necked, 10-mL round flask equipped with a magnetic stirring element. In parallel, a stopcock (ø14) was prepared connected to a cold trap mounted backwards. Next, a catalyst Cat. 5 was added (0.01 mol, 1.0 mol %). Immediately afterwards, the reaction flask was connected to a system with a collector connected to a membrane vacuum pump. After evacuating the reaction system, the cold trap was immersed in liquid nitrogen, and the reaction flask—in a heating bath at a temperature determined as 90° C. This was stirred for the next 24 hours, while maintaining the lowest possible pressure of 7-8 mbar indicated by the vacuum meter of the membrane vacuum pump. During the reaction, products were collected in the collector attached to the reaction flask. After 24 hours, the connection of the reaction system to the membrane vacuum pump was closed, the heating bath was removed, the stirring was stopped and the reaction system was opened so that air could access it. The crude product was eluted from the cold trap with diethyl ether (20 mL), then evaporated on a rotary evaporator at a pressure of 300 mbar at room temperature. 87 mg of the product was obtained with a purity of 99%, equivalent to a 79% yield. (Table 2, example 4)

### Example XXIII

### Macrocyclization of oxacycloocto-4-en-2-one

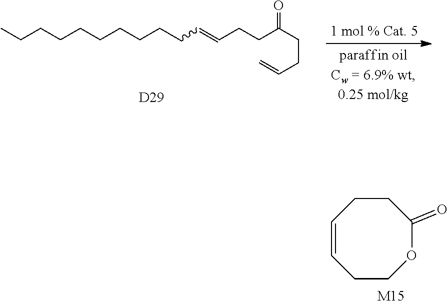

### Diagram 20. Macrocyclization of oxacycloocto-4-en-2-one

TABLE 15
Results of macrocyclization
of oxacycloocto-4-en-2-one.

| | Time [h] | Temp. [° C.] | vacuum [mbar] | Yield |
|---|---|---|---|---|
| 1 | 8 | 55 | 1 × 10⁻³ | 14% |

Procedure 1.

Paraffin oil (3 g, CAS: 8012-95-1, Sigma-Aldrich; filtered through a neutral Al₂O₃ gel) and a substrate for the ring metathesis reaction D30 (0.75 mmol, molar concentration in the reaction mixture=0.25 mol/kg) were placed in a single-necked, 10 mL round flask equipped with a magnetic stirring element. In parallel, a stopcock (ø14) was prepared connected to a double cold trap. Next, a catalyst Cat. 5 was added (7.5 μmol, 1.0 mol %). Immediately afterwards, the reaction flask was connected to a system with a collector connected to a rotary oil pump. After evacuating the reaction system, the cold trap was immersed in liquid nitrogen, and the reaction flask—in a heating bath at a temperature determined as 55° C. This was stirred for the next 8 hours, while maintaining the lowest possible pressure of 1×10⁻³ mbar indicated by the vacuum meter of the rotary oil pump. During the reaction, products were collected in the collector attached to the reaction flask. After 8 hours, the connection of the reaction system to the oil pump was closed, the heating bath was removed, the stirring was stopped and the reaction system was opened so that air could access it. The crude product was eluted from the cold trap with diethyl ether (20 mL), then evaporated on a rotary evaporator at a pressure of 300 mbar at room temperature. 67.7 mg of the product was obtained with a purity of 20%, equivalent to a 14% yield. (Table 15, example 1)

### Example XXIV

Macrocyclization of Cyclohexadecene.

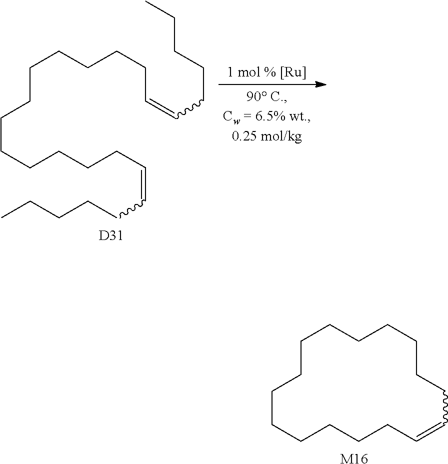

### Diagram 21. Macrocyclization of Cyclohexadecene

TABLE 16
Results for macrocyclization of
cyclohexadecene for various procedures.

| | [Ru] | diluent | time [h] | Yield |
|---|---|---|---|---|
| 1 | Cat. 5 | Paraffin oil | 8 | 16% |
| 2 | Cat. 5 | Paraffin oil | 24 | 14% |
| 3 | Cat. 4 | Paraffin oil | 24 | 15% |
| 4 | Cat. 4 | PAO6 | 24 | 22% |

Procedure 1.

A polyalphaolefin—PAO6 (2 g, filtered through a neutral Al₂O₃ gel) and a substrate for ring metathesis reaction D31 (0.5 mmol, molar concentration in the reaction mixture=0.25 mol/kg) were placed in a single-necked, 10-mL round flask equipped with a magnetic stirring element. In parallel, a stopcock (ø14) was prepared connected to a cold trap mounted backwards. Next, a catalyst Cat. 4 was added (5 μmol, 1.0 mol %). Immediately afterwards, the reaction flask was connected to a system with a collector connected to an oil pump. After evacuating the reaction system, the cold trap was immersed in liquid nitrogen, and the reaction flask—in a heating bath at a temperature determined as 90° C. This was stirred for the next 24 hours, while maintaining the lowest possible pressure of 1×10⁻³ mbar indicated by the vacuum meter of the rotary oil pump. During the reaction, products were collected in the collector attached to the reaction flask. After 24 hours, the connection of the reaction system to the oil pump was closed, the heating bath was removed, the stirring was stopped and the reaction system was opened so that air could access it. The crude product was eluted from the cold trap with diethyl ether (20 mL), then evaporated on a rotary evaporator at a pressure of 20 mbar at 37° C. (water bath temperature). 124.7 mg of the product was obtained with a purity of 20%, equivalent to a 22% yield. (Table 16, example 4)

Procedure 2.

Paraffin oil (2 g, CAS: 8012-95-1, Sigma-Aldrich; filtered through a neutral Al₂O₃ gel) and a substrate for the ring metathesis reaction D31 (0.5 mmol, molar concentration in the reaction mixture=0.25 mol/kg) were placed in a single-necked, 10 mL round flask equipped with a magnetic stirring element. In parallel, a stopcock (ø14) was prepared connected to a cold trap mounted backwards. Next, a catalyst Cat. 4 was added (5 μmol, 1.0 mol %). Immediately afterwards, the reaction flask was connected to a system with a collector connected to an oil pump. After evacuating the reaction system, the cold trap was immersed in liquid nitrogen, and the reaction flask—in a heating bath at a temperature determined as 90° C. This was stirred for the next 24 hours, while maintaining the lowest possible pressure of 1×10⁻³ mbar indicated by the vacuum meter of the rotary oil pump. During the reaction, products were collected in the collector attached to the reaction flask. After 24 hours, the connection of the reaction system to the oil pump was closed, the heating bath was removed, the stirring was stopped and the reaction system was opened so that air could access it. The crude product was eluted from the cold trap with diethyl ether (20 mL), then evaporated on a rotary evaporator at a pressure of 20 mbar at 37° C. (water bath temperature). 102 mg of the product was obtained with a purity of 17%, equivalent to a 15% yield. (Table 16, example 3)

Procedure 3.

Paraffin oil (2 g, CAS: 8012-95-1, Sigma-Aldrich; filtered through a neutral Al₂O₃ gel) and a substrate for the ring metathesis reaction D31 (0.5 mmol, molar concentration in the reaction mixture=0.25 mol/kg) were placed in a single-necked, 10 mL round flask equipped with a magnetic stirring element. In parallel, a stopcock (ø14) was prepared connected to a cold trap mounted backwards. Next, a catalyst Cat. 5 was added (5 μmol, 1.0 mol %). Immediately afterwards, the reaction flask was connected to a system with a collector connected to an oil pump. After evacuating the reaction system, the cold trap was immersed in liquid nitrogen, and the reaction flask—in a heating bath at a temperature determined as 90° C. This was stirred for the next 24 hours, while maintaining the lowest possible pressure of 1×10³ mbar indicated by the vacuum meter of the rotary oil pump. During the reaction, products were collected in the collector attached to the reaction flask. After 24 hours, the connection of the reaction system to the oil pump was closed, the heating bath was removed, the stirring was stopped and the reaction system was opened so that air could access it. The crude product was eluted from the cold trap with diethyl ether (20 mL), then evaporated on a rotary evaporator at a pressure of 20 mbar at 37° C. (water bath temperature). 98.8 mg of the product was obtained with a purity of 16%, equivalent to a 14% yield. (Table 16, example 2)

Procedure 4.

Paraffin oil (4 g, CAS: 8012-95-1, Sigma-Aldrich; filtered through a neutral Al₂O₃ gel) and a substrate for the ring metathesis reaction D31 (1 mmol, molar concentration in the reaction mixture=0.25 mol/kg) were placed in a single-necked, 10 mL round flask equipped with a magnetic stirring element. Next, a catalyst Cat. 5 was added (1 mmol, 1.0 mol %). Immediately afterwards, the reaction flask was connected to a system with a collector connected to a membrane vacuum pump. After evacuating the reaction system, the reaction flask was immersed in a heating bath at a temperature determined as 90° C. This was stirred for the next 8 hours, while maintaining the lowest possible pressure of 7-8 mbar indicated by the vacuum meter of the membrane vacuum pump. During the reaction, products were collected in the collector attached to the reaction flask. After 8 hours, the connection of the reaction system to the membrane vacuum pump was closed, the heating bath was removed, the stirring was stopped and the reaction system was opened so that air could access it. The crude product was eluted from the collector with diethyl ether (20 mL), then evaporated on a rotary evaporator at a pressure of 300 mbar at room temperature. 211 mg of the product was obtained with a purity of 16%, equivalent to a 16% yield. (Table 16, example 1)

### Example XXV

### Metathesis Cyclization of 7-oct-1-enyl Oleate Using Paraffin Oil as a Diluent with a Temperature Gradient of 40->110° C.

General Procedure of Metathesis Cyclization:

Paraffin oil (2 g, CAS: 8012-95-1, Sigma-Aldrich; filtered through a neutral Al₂O₃ gel) and a substrate for the ring metathesis reaction (0.5 mmol, molar concentration in the reaction mixture=0.25 mol/kg) were placed in a single-necked, 10 mL round flask equipped with a magnetic stirring element. Next, catalyst Cat. 4 was added. Immediately afterwards, the reaction flask was connected to a system with a collector connected to a diffusion pump. After evacuating the reaction system, the reaction flask was immersed in a heating bath at a temperature determined as 40° C. The temperature was gradually increased to 110° C. over 30 minutes. The stirring at the determined temperature of 110° C. was continued for the next 7 hours and 30 minutes while maintaining the lowest pressure possible. The vacuum level was read from the vacuum meter connected directly to the diffusion pump (the vacuum was of the order of 10⁻⁶). During the reaction, products were collected in the collector attached to the reaction flask. After 8 hours, the connection of the reaction system to the diffusion pump was closed, the heating bath was removed, the stirring was stopped and the reaction system was opened so that air could access it. The crude product was eluted from the collector with hexane (20 mL), and then the solvents were evaporated under reduced pressure and purified by chromatography using n-hexane and ethyl acetate.

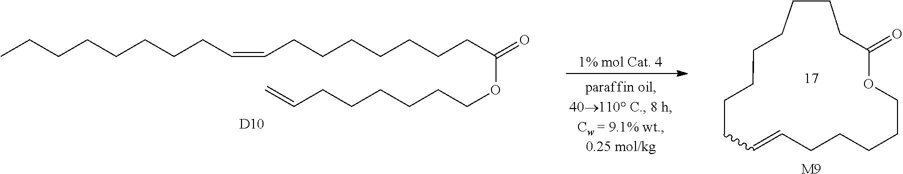

### Diagram 22. Metathesis cyclization of 7-oct-1-enyl oleate

Yield=78%, purity=87%, E/Z ratio=2.1

### Example XXVI

### Metathesis Cyclization of 7-oct-1-enyl Oleate Using PAO 6 as a Diluent with a Temperature Gradient of 40->110° C.

General Procedure of Metathesis Cyclization:

PAO 6 (2 g, filtered through a neutral Al₂O₃ gel) and a substrate for the ring metathesis reaction (0.5 mmol, molar concentration in the reaction mixture=0.25 mol/kg) we placed in a single-necked, 10-mL round flask equipped with a magnetic stirring element. Next, catalyst Cat. 4 was added. Immediately afterwards, the reaction flask was connected to a system with a collector connected to a diffusion pump. After evacuating the reaction system, the reaction flask was immersed in a heating bath at a temperature determined as 40° C. The temperature was gradually increased to 110° C. over 30 minutes. The stirring at the determined temperature of 110° C. was continued for the next 7 hours and 30 minutes while maintaining the lowest pressure possible. The vacuum level was read from the vacuum meter connected directly to the diffusion pump (the vacuum was of the order of 10⁻⁶). During the reaction, products were collected in the collector attached to the reaction flask. After 8 hours, the connection of the reaction system to the diffusion pump was closed, the heating bath was removed, the stirring was stopped and the reaction system was opened so that air could access it. The crude product was eluted from the collector with hexane (20 mL), and then the solvents were evaporated under reduced pressure and purified by chromatography using n-hexane and ethyl acetate.

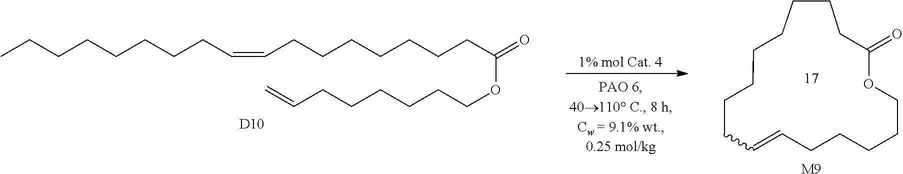

### Diagram 23. Metathesis cyclization of 7-oct-1-enyl oleate

Yield=86%, purity=94%, E/Z ratio=2.1

### Example XXVII

### Metathesis Cyclization of 3-but-1-enyl Oleate Using Paraffin Oil as a Diluent and a Diffusion Pump

The reaction was conducted in accordance with the procedure and conditions of example XIII (i)

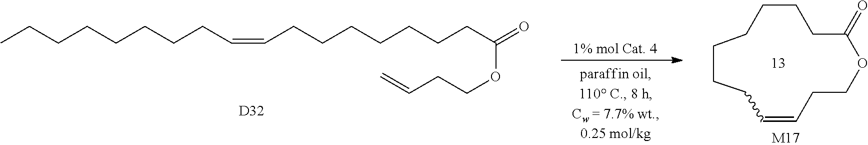

### Diagram 24. Metathesis cyclization of 3-but-1-enyl oleate

Yield=43%, purity=93%, E/Z ratio=2.4

### Example XXVIII

### Metathesis Cyclization of 3-but-1-enyl Oleate Using Paraffin Oil as a Diluent and a Rotary Oil Pump

The reaction was conducted in accordance with the procedure of example XX using paraffin oil as diluent

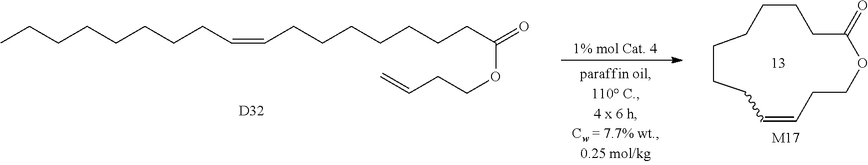

### Diagram 25. Metathesis cyclization of 3-but-1-enyl oleate

Yield=25%, purity=67%, E/Z ratio=2.5

### Example XXIX

### Metathesis Cyclization of 3-but-1-enyl Oleate Using PAO 6 as a Diluent and a Rotary Oil Pump

The reaction was conducted in accordance with the procedure of example XX using PAO 6 as diluent.

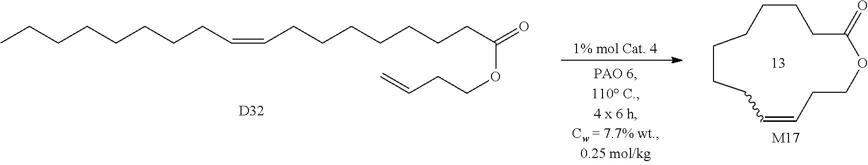

### Diagram 26. Metathesis cyclization of 3-but-1-enyl oleate

Yield=25%, purity=95%, E/Z ratio=2.8

### Experimental Part

11-docosan

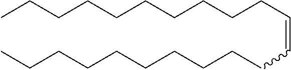 [SMILES: CCCCCCCCCCC=CCCCCCCCCCC]

1-dodecene (1 eq.) and tetrafluorobenzoquinone (0.5 mol %) were placed in a 250 mL single-necked round flask and then heated to 80° C. Catalyst cat. 13 (500 ppm) was dissolved in 1 mL of methylene chloride and added to the flask. The reaction mixture was heated for 30 minutes, after which it was cooled to room temperature and SnatchCat solution was added. After 30 minutes, the contents of the flask were diluted with n-hexane and filtered through SiO₂, eluting with pure n-hexane. The filtrate was evaporated and then dried using rotary oil pump A colourless oil was obtained that solidified at room temperature. 97% yield.

¹H NMR (400 MHz, CDCl₃) 5.45-5.29 (m, 2H), 2.08-1.87 (m, 4H), 1.41-1.15 (m, 32H), 0.95-0.80 (m, 6H); ¹³C NMR (400 MHz, CDCl₃) 130.51, 130.05, 32.78, 32.09, 29.94, 29.82, 29.80, 29.73, 29.70, 29.52, 29.48, 29.33, 27.36, 22.86, 14.30.

### 4-pentadecanoic acid (K6)

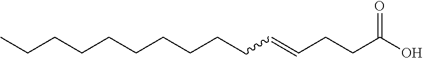 [SMILES: CCCCCCCCCCC=CCCC(=O)O]

Acid K6 (1 eq.) and 11-docosan (5 eq.) were dissolved in 2 mL of dry methylene chloride in a single-necked round flask under the atmosphere of argon. Tetrafluorobenzoquinone (5.5 mol %) was then added, and the reaction mixture was heated to 40° C. Catalyst cat. 18 (2 mol %) was added in ten portions every 30 minutes. After 5 hours, the mixture was cooled to room temperature and SnatchCat solution was added. The product was purified by chromatography using 20% EtOAc in n-hexane. An orange solid was obtained. 92% yield.

¹H NMR (400 MHz, CDCl₃) δ 5.53-5.29 (m, 2H), 2.45-2.28 (m, 4H), 2.07-1.93 (m, 2H), 1.39-1.21 (m, 17H), 0.92-0.84 (m, 3H); ¹³C NMR (101 MHz, CDCl₃) δ 132.36, 132.08, 127.60, 127.01, 34.12, 32.65, 32.07, 29.80, 29.78, 29.71, 29.66, 29.57, 29.50, 29.46, 29.29, 27.74, 27.37, 22.85, 22.66, 14.28.

### methyl 3-oxo-2-((Z)-hexadec-7-en-1-yl)-(Z)-11-eikosate (D27)

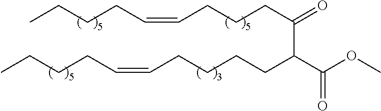

TiCl₄ (1.5 eq.) dissolved in 4 mL of dry toluene was added dropwise under an inert gas atmosphere to a mixture of methyl oleate (1.0 eq.) and NBu₃ (18 eq.) in 16 mL of dry toluene at 0 to 5° C. The temperature was maintained for one hour after the dropwise addition was over, and then 20 mL of water was added and washed twice with diethyl ether. The combined ether layers were dried over Na₂SO₄. The product was purified by chromatography using 2.5% Et₂O in n-hexane. A yellow oil was obtained. 93% yield.

¹H NMR (400 MHz, CDCl₃) δ 5.40-5.27 (m, 4H), 3.71 (s, 3H), 3.43 (t, J=7.4 Hz, 1H), 2.61-2.39 (m, 2H), 2.07-1.95 (m, 8H), 1.90-1.74 (m, 2H), 1.64-1.50 (m, 2H), 1.40-1.17 (m, 40H), 0.93-0.83 (m, 6H); ¹³C NMR (101 MHz, CDCl₃) δ 205.63, 170.58, 130.19, 130.14, 129.87, 129.79, 59.16, 52.43, 42.02, 32.06, 29.91, 29.85, 29.77, 29.68, 29.48, 29.44, 29.42, 29.26, 29.16, 29.12, 28.43, 27.63, 27.37, 27.32, 27.27, 23.59, 22.84, 14.28.

### (9Z,26Z)-pentatriakonta-9,26-dien-18-on (D28)

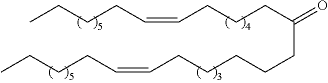

1 equivalent of diene D27 was dissolved in the mixture of THF-MeOH-5% NaOH solution. The reaction mixture was heated for 5 hours at a temperature of 70° C. It was then cooled to 0° C. and H₂SO₄ was added until the reaction was slightly acidic. After evaporation of the solvents, the mixture was washed twice with diethyl ether. The combined ether layers were dried over Na₂SO₄. The product was purified by chromatography using 2% EtOAc in n-hexane. A yellow oil was obtained. 92% yield.

¹H NMR (400 MHz, CDCl₃) δ 5.40-5.28 (m, 4H), 2.37 (t, J=7.6 Hz, 4H), 2.05-1.95 (m, 8H), 1.62-1.49 (m, 4H), 1.40-1.19 (m, 40H), 0.93-0.83 (m, 6H); ¹³C NMR (101 MHz, CDCl₃) δ 211.81, 130.12, 129.89, 42.96, 32.06, 29.92, 29.85, 29.68, 29.48, 29.40, 29.27, 27.36, 27.31, 24.02, 22.84, 14.28.

### eikosa-1,8-dien-5-one (D29)

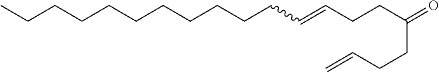 [SMILES: C=CCCC(=O)CCC=CCCCCCCCCCCC]

A suitable carboxylic acid K61 (1 eq.) was dissolved in anhydrous methylene chloride under an inert gas atmosphere. A few drops of N,N-dimethylformamide were added, and then oxalyl chloride (1.2 eq.) was added dropwise at room temperature. Gas emission and change of colour to yellow were observed. After an hour, substrate conversion was checked using ¹H NMR. Methylene chloride and unreacted oxalyl chloride were evaporated using a membrane vacuum pump. 25 mL of THF was added to the resulting oil, after which the evaporation of the solvent was repeated. The resulting acid chloride was then placed in a 500 mL three-necked round flask, dissolved in 270 mL THF. After cooling the mixture to −78° C., Fe(acac)₃ (5 mol %) was added in one portion. After dissolving the catalyst, a homoallyl magnesium bromide solution (0.77 M in THF, 17 mL) was added dropwise to the mixture and allowed to rest while stirring for 30 minutes, after which 20 mL of saturated ammonium chloride solution was added and allowed to rest while stirring until it reached room temperature. After evaporation of the solvents, the mixture was washed four times with methyl tert-butyl ether. The combined ether layers were dried over MgSO₄. The product was purified by chromatography using 10% EtOAc in n-hexane. A white solid was obtained. 83% yield.

¹H NMR (400 MHz, CDCl₃) δ 5.87-5.73 (m, 1H), 5.39 (dd, J=7.9, 5.7 Hz, 2H), 5.20-4.92 (m, 2H), 2.56-2.40 (m, 4H), 2.38-2.19 (m, 4H), 2.07-1.89 (m, 2H), 1.43-1.19 (m, 18H), 0.93-0.81 (m, 3H); ¹³C NMR (101 MHz, CDCl₃) δ 209.93, 137.33, 131.80, 131.46, 128.35, 127.80, 115.34, 115.31, 42.91, 42.04, 32.66, 32.06, 29.80, 29.78, 29.70, 29.66, 29.62, 29.49, 29.47, 29.31, 27.90, 27.87, 27.34, 26.97, 22.84, 21.80, 14.27.

Obtained by the Following:

General Method for the Preparation of Diene Esters (Dx), Substrates for the RCM Reaction

### but-3-en-1-yl pentadeca-4-enoate (D30)

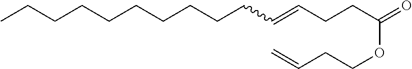 [SMILES: C=CCCOC(=O)CCC=CCCCCCCCCCC]

¹H NMR (400 MHz, CDCl₃) δ 5.78 (ddt, J=17.0, 10.2, 6.7 Hz, 1H), 5.52-5.28 (m, 2H), 5.17-5.03 (m, 2H), 4.13 (td, J=6.8, 1.9 Hz, 2H), 2.45-2.24 (m, 6H), 2.07-1.91 (m, 2H), 1.38-1.19 (m, 16H), 0.92-0.84 (m, 3H); ¹³C NMR (101 MHz, CDCl₃) δ 173.39, 134.21, 132.03, 131.72, 127.99, 127.42, 117.32, 117.30, 63.53, 63.48, 34.54, 33.25, 32.67, 32.07, 29.79, 29.71, 29.67, 29.61, 29.50, 29.47, 29.30, 28.10, 27.36, 22.98, 22.85, 14.28.

### hexadeceno-1,16-diol

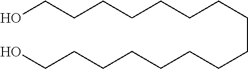 [SMILES: OCCCCCCCCCCCCCCCCO]

100 mL of THE and 7 equivalents of lithium aluminium hydride were placed in a 250 mL single-necked round flask and then cooled to a temperature of 0° C. The whole was stirred and then 1 equivalent of hexadecandioic acid was added portionwise. The reaction mixture was heated to room temperature and then heated at THE reflux for 4 hours. Afterwards, the reaction mixture was cooled to 0° C. and diluted with diethyl ether to a volume of 200 mL. Next, the following solutions were added dropwise: 9.8 mL water, 9.8 mL of a 15% solution of NaOH (aq), 29.4 mL water. The mixture was allowed to heat to room temperature and was stirred for 15 minutes. Next, MgSO₄ was added and the mixture was allowed to rest while stirring for another 15 followed by methylene chloride. The resulting filtrate was evaporated and then dried to yield 7.27 grams of diol. 81% yield.

¹H NMR (400 MHz, CD₃OD) δ 3.52 (t, J=6.6 Hz, 4H), 3.31-3.29 (m, 4H), 1.39-1.25 (m, 24H); ¹³C NMR (101 MHz, CD₃OD) δ 61.58, 32.25, 29.36, 29.35, 29.32, 29.20, 25.53.

### Octacose-6,22-dien (D31)

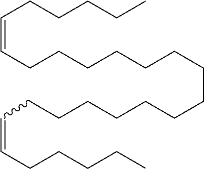 [SMILES: CCCCCC=CCCCCCCCCCCCCCC/C=C\CCCCC]

The diol (1 eq.) and 100 mL acetonitrile were placed in a 200 mL beaker with a stirring element. The mixture was re-heated to a temperature of 50° C., after which copper complex [Cu(MeCN)₄]PF₆ (10 mol %), TEMPO (10 mol %) 2,2′-bipyridyl (10 mol %) and 1-methylimidazole (20 mol %) were added one by one. The reaction was conducted for 4 hours until the substrate disappeared. Afterwards, the whole was evaporated and dissolved in methylene chloride, and SnachCat solution was then added. After stirring, the solution was filtered (through SiO₂), washing it with methylene chloride, and then the solution was evaporated.

Hexyl triphenylphosphine bromide (2.1 eq.) was dissolved in 120 mL tetrahydrofuran under the atmosphere of argon in a 250 mL single-necked round flask. 2.5M solution of n-butyllithium in hexane (2.1 eq) was added dropwise to the resulting mixture, and allowed to rest while stirring for 30 minutes. The resulting solution was cooled to 0° C., and the previously obtained aldehyde in a solution of tetrahydrofuran (1 eq.) was slowly added dropwise. The reaction mixture was heated to room temperature and then stirring was continued for another 2 hours at room temperature. 50 mL of saturated ammonium chloride solution was then added. The mixture was washed four times with methylene chloride. The combined ether layers were dried over MgSO₄. The product was purified by chromatography using n-hexane. A colourless oil was obtained. 75% yield.

¹H NMR (400 MHz, CDCl₃) 5.46-5.27 (m, 4H), 2.10-1.90 (m, 8H), 1.44-1.16 (m, 36H), 0.97-0.80 (m, 6H); ¹³C NMR (101 MHz, CDCl₃) δ 130.05, 130.04, 31.69, 29.93, 29.85, 29.82, 29.73, 29.62, 29.48, 27.36, 27.33, 22.75, 14.25.

### But-3-en-1-yl oleate (D32)

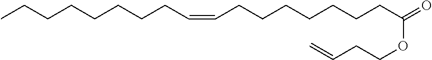 [SMILES: C=CCCOC(=O)CCCCCCC/C=C\CCCCCCCC]

Oleic acid K3 (1 eq.) and 3-buten-1-ol A10 (1.5 eq.) were dissolved in toluene (20 mL), after which two drops of sulfuric acid were added. The Dean-Stark apparatus was then mounted and the mixture was heated for 3 hours under reflux. After cooling to room temperature, the reaction mixture was transferred to a separator, washed with saturated sodium bicarbonate solution (1×5 mL) and brine (1×5 mL). The organic fraction was concentrated on a rotary evaporator. The crude product was purified by column chromatography with 2% EtOAc/n-hexane (v/v). A colourless oil was obtained with a 87% yield.

¹H NMR (400 MHz, CDCl₃) δ 5.78 (ddt, J=16.9, 10.2, 6.7 Hz, 1H), 5.39-5.28 (m, 2H), 5.15-5.02 (m, 2H), 4.12 (t, J=6.8 Hz, 2H), 2.42-2.34 (m, 2H), 2.29 (t, J=7.5 Hz, 2H), 2.05-1.96 (m, 4H), 1.67-1.56 (m, 2H), 1.38-1.20 (m, 20H), 0.94-0.83 (m, 3H); ¹³C NMR (101 MHz, CDCl₃) δ 13C NMR (101 MHz, cdcl3) δ 174.0, 134.2, 130.1, 129.9, 117.3, 63.4, 34.5, 33.3, 32.1, 29.9, 29.8, 29.7, 29.5, 29.3, 29.3, 27.4, 27.3, 25.1, 22.8, 14.3.

### cycloheptadec-9-en-1-on (M13)

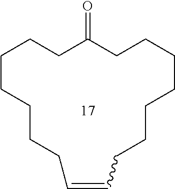 [SMILES: O=C1CCCCCCCC=CCCCCCCC1]

¹H NMR (400 MHz, CDCl₃) δ 5.38-5.23 (m, 2H), 2.37 (dt, J=9.4, 6.8 Hz, 4H), 1.98 (ddt, J=8.2, 3.9, 2.4 Hz, 4H), 1.69-1.51 (m, 4H), 1.43-1.13 (m, 16H); ¹³C NMR (100 MHz, CDCl₃) δ 213.31, 212.68, 131.07, 130.25, 42.59, 42.56, 32.06, 29.14, 28.93, 28.89, 28.72, 28.47, 28.33, 28.24, 27.52, 26.80, 24.13, 23.98.

### cyclohept-4-en-1-on (M14)

¹H NMR (400 MHz, CDCl₃) δ 5.78 (t, J=2.9 Hz, 2H), 2.71-2.60 (m, 4H), 2.43-2.28 (m, 4H); ¹³C NMR (100 MHz, CDCl₃) δ 213.8, 129.6, 42.6, 24.2.

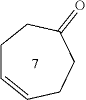

### (9-E/Z)-dodec-9-en-12-olid (M17)

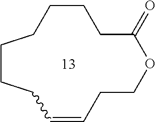 [SMILES: O=C1CCCCCCCC=CCCO1]

¹H NMR (400 MHz, CDCl₃) δ 5.60-5.25 (m, 2H), 4.24-4.08 (m, 2H), 2.45-2.23 (m, 4H), 2.13-1.94 (m, 2H), 1.72-1.57 (m, 2H), 1.53-1.10 (m, 8H); ¹³C NMR (101 MHz, CDCl₃) δ ¹³C NMR (101 MHz, cdcl3) δ 174.8, 174.1, 134.8, 134.8, 132.3, 127.2, 126.5, 64.3, 63.0, 35.5, 34.0, 32.7, 32.7, 32.1, 29.8, 27.6, 27.5, 27.4, 27.4, 27.4, 27.4, 27.3, 27.0, 26.1, 26.0, 24.7, 24.3, 23.6.

### Example XXX (Complementing Example XII to Table 8)

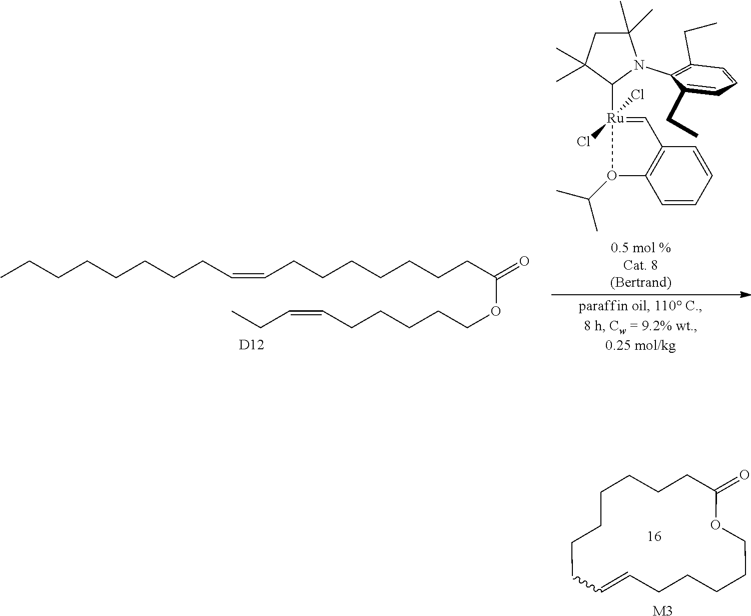

### Diagram 27. Metathesis cyclization of cis-non-6-en-1-yl oleate using Bertrand Complex

Yield=42%, purity=24%, E/Z ratio=2.9

### Example XXXI (Complementing Example XII to Table 8)

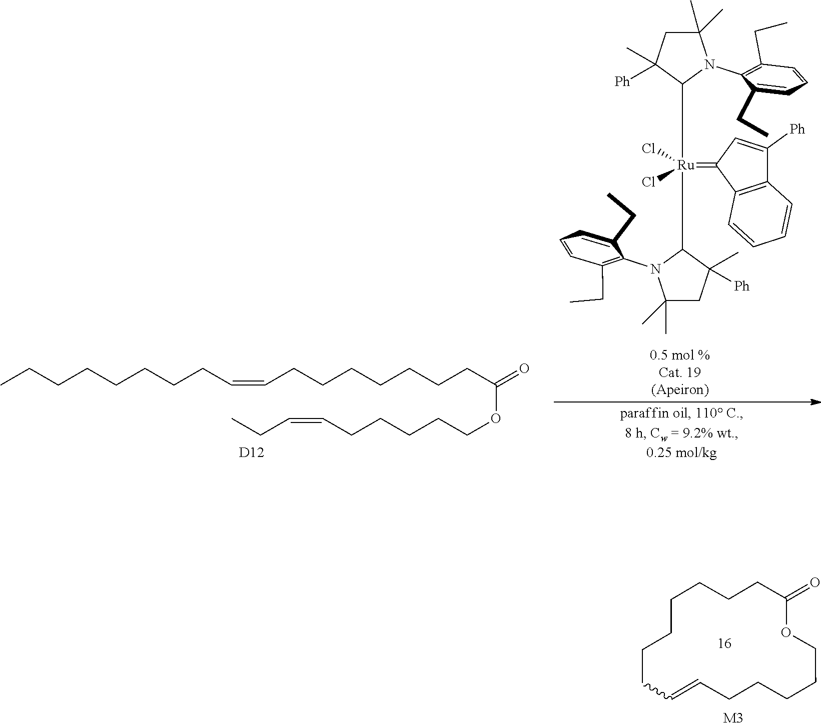

### Diagram 28. Metathesis cyclization of cis-non-6-en-1-yl oleate Using a Complex of cat. 19

Yield=29%, purity=38%, E/Z ratio=2.8

### Example XXXI

### Metathesis Cyclization of 3-but-1-enyl Oleate Using PAO 6 as a Diluent with a Temperature Gradient of 40->110° C. and a Diffusion Pump

The reaction was conducted in accordance with the procedure and conditions of example XXVI

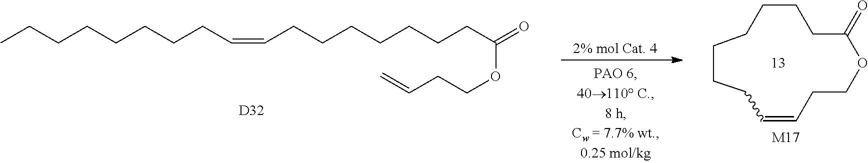

### Diagram 29. Metathesis cyclization of 3-but-1-enyl oleate

Yield=73%, purity=96%, E/Z ratio=3.6

### Example XXXII

### Metathesis Cyclization of cis-non-6-en-1-yl Oleate Using PAO 6 as a Diluent and a Diffusion Pump in a Weight Concentration of 90% w/w

PAO6 (60 mg) and cis-non-6-en-1-yl oleate (D12, 610 mg; 1.5 mmol) were placed in a single-necked, 10 mL round flask equipped with a magnetic stirring element. Next, catalyst cat. 4 was added in the amount of 1% mol. Immediately afterwards, the reaction flask was connected to a system with a collector connected to a diffusion pump. After evacuating the reaction system, the reaction flask was immersed in a heating bath at a temperature determined as 110° C. This was stirred for the next 8 hours, while maintaining the lowest possible pressure (of the order of 1·10⁻⁶ mbar). During the reaction, products were collected in the collector attached to the reaction flask. After 8 hours, the connection of the reaction system to the diffusion flask was closed, the heating bath was removed, the stirring was stopped and the reaction system was opened so that air could access it. The crude product was eluted from the collector with hexane (20 mL), and then purified by chromatography using n-hexane and ethyl acetate.

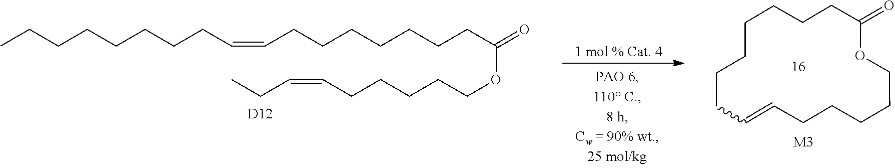

### Diagram 29. Metathesis Cyclization of cis-non-6-en-1-yl oleate at a Concentration of 90% w/w

Yield=34%, purity=83%, E/Z ratio=2.7

1-20. (canceled)

21. A method for producing a cyclic compound Mx, 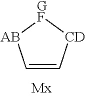
wherein:
AB and CD are each independently a group selected from C₁-C₁₀ alkyl, C₂-C₂₅ alkenyl;
GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom; 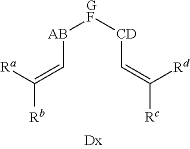
characterised in that olefin Dy with the formula 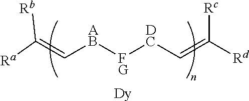
wherein:
AB and CD are each independently a group selected from such as a hydrogen atom, C₁-C₁₀ alkyl, C₁-C₁₀ perfluoroalkyl, C₃-C₇ cycloalkyl, C₂-C₂₅ alkenyl, C₂-C₂₅ perfluoroalkenyl, C₃-C₂₅ cycloalkenyl, C₂-C₂₅ alkinyl, C₂-C₂₅ perfluoroalkinyl, C₃-C₂₅ cycloalkinyl, C₅-C₂₄ aryl, C₅-C₂₀ heteroaryl, C₅-C₂₄ perfluoroaryl, 3-12-membered heterocycle, which may be optionally substituted independently with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;
R^{a}, R^{b}, R^{c} i R^{d} are each independently a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;
GF is an ether (—O—), ester (—C(O)O—), carbonyl (—C(O)—), amido (—C(O)NR—), malonate (—C(COOR)₂—) group, wherein R is independently a hydrogen atom, C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;
are subjected to olefin metathesis reaction with the compound of the formula 1, 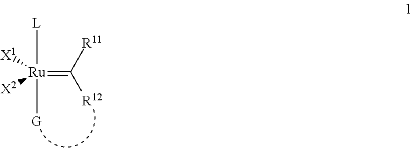
wherein:
X¹ and X² are each independently an anionic ligand selected from such as halogen atoms, —CN, —SCN, —OR′, —SR′, —O(C═O)R′, —O(SO₂)R′, —OSi(R′)₃ group, wherein R′ is C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy, or a halogen atom;
R¹¹, R¹² are each independently a hydrogen atom, a halogen atom, optionally substituted C₁-C₂₅ alkyl, optionally substituted C₁-C₂₅ perfluoralkyl, optionally substituted C₂-C₂₅ alkene, optionally substituted C₃-C₇ cycloalkyl, optionally substituted C₂-C₂₅ alkenyl, optionally substituted C₃-C₂₅ cycloalkenyl, optionally substituted C₂-C₂₅ alkinyl, optionally substituted C₃-C₂₅ cycloalkinyl, optionally substituted C₁-C₂₅ alkoxy, optionally substituted C₅-C₂₄ aryloxy, optionally substituted C₅-C₂₀ heteroaryloxy, optionally substituted C₅-C₂₄ aryl, optionally substituted C₅-C₂₀ heteroaryl, optionally substituted C₇-C₂₄ aralkyl, optionally substituted C₅-C₂₄ perfluoroaryl, optionally substituted 3-12-membered heterocycle;
wherein the substituents R¹¹ and R¹² may be interconnected to form a ring selected from the group consisting of C₃-C₇ cycloalkyl, C₃-C₂₅ cycloalkenyl, C₃-C₂₅ cycloalkinyl, C₅-C₂₄ aryl, C₅-C₂₀ heteroaryl, C₅-C₂₄ perfluoroaryl, 3-12-membered heterocycle, each of which may be substituted with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle, in which case the dotted line between G and R¹² is not a chemical bond;
wherein the substituents R¹¹ i R¹² preferably are a hydrogen atom or aryl independently substituted with the following groups: alkoxy (—OR′), sulfide (—SR′), sulfoxide (—S(O)R′), sulfonium (—S⁺R′₂), sulphonic (—SO₂R′), sulfonamide (—SO₂NR′₂), amino (—NR′₂), ammonium (—N⁺R′₃), nitro (—NO₂), cyano (—CN), phosphonium (—P(O)(OR′)₂), phosphinium (—P(O)R′(OR′)), phosphonous (—P(OR′)₂), phosphine (—PR′₂), phosphine oxides (—P(O)R′₂), phosphonium (—P⁺R′₃), carboxy (—COOH), ester (—COOR′), amide (—CONR′₂), amide (—NR′COR″), formyl (—CHO), ketone (—COR′), in which groups R′ is C₁-C₅ alkyl, C₁-C₅ perfluoroalkyl, C₅-C₂₄ aryl, C₇-C₂₄ aralkyl, C₅-C₂₄ perfluoroaryl;
L is selected from such as:
a) 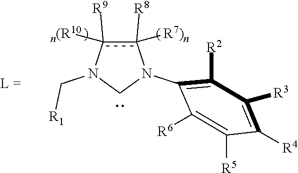
wherein:
R¹ is a heteroaryl group;
R², R³, R⁴, R⁵, R⁶ are each independently a hydrogen atom, a C₁-C₂₅ alkyl group, a C₁-C₂₅ alkoxy group or a C₂-C₂₅ alkenyl group, wherein the substituents R², R³, R⁴, R⁵, R⁶ may be interconnected to form a substituted or unsubstituted cyclic C₄-C₁₀ or polycyclic C₄-C₁₂ system;
R⁷, R¹, R⁹, i R¹⁰ are each independently a hydrogen atom or a C₁-C₂₅ alkyl group, R⁷ and/or R⁸ can be connected with R⁹ and/or R¹⁰ to form a cyclic system;
n is 0 or 1;
b) 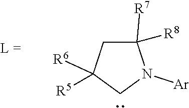
wherein:
Ar is an aryl group which is substituted with hydrogen atoms or is optionally substituted with at least one of the following groups: C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, C₇-C₂₄ aralkyl, C₅-C₂₄ perfluoroaryl, or a halogen atom;
R⁵, R⁶, R⁷ and R⁸ are each independently a hydrogen atom or one of the following groups: C₁-C₂₅ alkyl, C₃-C₁₂ cycloalkyl, C₁-C₅ perfluoroalkyl, C₂-C₁₂ alkenyl, C₅-C₂₀ aryl, C₅-C₂₄ aryloxy, C₂-C₂₀ heterocyclo, C₄-C₂₀ heteroarylo, C₅-C₂₀ heteroaryloxy, C₇-C₂₄ aralkyl, C₅-C₂₄ perfluoroaryl, which are optionally substituted with at least one C₁-C₁₂ alkyl, C₁-C₁₂ perfluoroalkyl, C₁-C₁₂ alkoxy, C₅-C₂₄ aryloxy, C₄-C₂₀ heteroaryl, C₅-C₂₀ heteroaryloxy, or a halogen atom; moreover, R⁵ and R⁶ and/or R⁷ i R′ may be interconnected to form a cyclic system;
c) 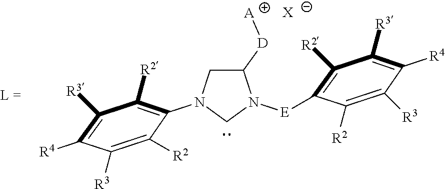
wherein:
the combination A⁺X⁻ and D is a hydrogen atom, or
A is independently a substituent containing a tertiary amine group or a quaternary ammonium group, which may be a N(R¹)(R²) or N⁺(R¹)(R²)(R³) group, wherein R¹, R² and R³ are each independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl; alternatively, A is one of the following groups: C₁-C₂₅ cycloaminoalkyl, C₁-C₂₅ cyclodiaminoalkyl, C₁-C₂₅ cyclotriaminoalkyl, C₁-C₂₅ cyclotetraaminoalkyl, C₁-C₂₅ cycloaminoammonioalkyl, wherein the at least one nitrogen atom is independently substituted with at least one R¹ group, wherein R¹ is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl so that at least one nitrogen atom in the ring forms a quaternary ammonium group;
X is independently a halogen atom, or CF₃SO₃⁻, BF₄⁻, PF₆⁻, and ClO₄⁻;
D is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl, or C₂-C₂₅ alkenyl, C₁-C₂₅ α,ω-dialkoxy, (CH₂CH₂O)ₙ, polyether, where n comprises from 1 to 25, a C₁-C₂₅ thioalkyl group, a C₁-C₂₅ α,ω-dithioalkyl group, a C₁-C₂₅ α,ω-diheteroalkyl group, a C₁-C₂₅ aminoalkyl group;
E is a single bond or independently a C₁-C₂₅ alkyl group, a C₁-C₁₂ perfluoroalkyl group, a C₃-C₇ cycloalkyl group, a C₁-C₂₅ alkoxy group;
X¹ and X² are each independently an anion ligand selected from such as halogen atoms;
R², R^{2′}, R³, R^{3′} i R⁴ are each independently a hydrogen atom, a halogen atom, a C₁-C₂₅ alkyl group, a C₃-C₇ cycloalkyl group, a C₁-C₂₅ alkoxy group, a C₅-C₂₄ perfluoroaryl group, a C₅-C₂₀ heteroaryl group or a C₂-C₂₅ alkenyl group, wherein the substituents R², R^{2′}, R³, R^{3′} and R⁴ may be interconnected to form a substituted or unsubstituted cyclic C₄-C₁₀ or polycyclic C₄-C₁₂ system;
and
G is selected from the substituents L listed above or G is a heteroatom selected from the group comprising an oxygen, nitrogen, sulphur, phosphorus, fluorine, chlorine, bromine and iodine atom, optionally substituted with a group selected from such as hydrogen atom C₁-C₂₅ alkyl, C₁-C₂₅ perfluoroalkyl, C₃-C₇ cycloalkyl, C₅-C₂₄ aryl, C₅-C₂₄ perfluoroaryl, C₅-C₂₀ heteroaryl, C₇-C₂₄ aralkyl, 3-12 membered heterocycle, the following groups: —COR′ acyl, (—CN) cyano, (—COOH) carboxy, (—COOR′) ester, (—CONR′₂) amide, (—SO₂R′) sulfonic, (—CHO) formyl, (—SO₂NR′₂) sulfonamide, (—COR′) ketone, wherein the R′ group is C₁-C₅ alkyl, C₁-C₅ perfluoroalkyl, C₅-C₂₄ aryl, C₅-C₂₄ perfluoroaryl, C₇-C₂₄ aralkyl, in which case the dotted line is a direct bond between the heteroatom and the R¹² substituent, in the form of an aryl optionally substituted by 1-4 substituents independently selected from the group comprising a hydrogen atom, a halogen atom, C₁-C₂₅ alkyl, C₁-C₂₅ perfluoroalkyl, C₂-C₂₅ alken, C₃-C₇ cycloalkyl, C₂-C₂₅ alkenyl, C₃-C₂₅ cycloalkenyl, C₂-C₂₅ alkinyl, C₃-C₂₅ cycloalkinyl, C₅-C₂₄ aryl, C₇-C₂₄ aralkyl, C₅-C₂₄ perfluoroaryl, C₅-C₂₀ heteroaryl, 3-12-membered heterocycle, one of the following groups: (—OR′) alkoxy, (—SR′) sulfide, (—NO₂) nitro, (—CN) cyano, (—COOH) carboxy, (—COOR′) ester, (—CONR′₂) amide, (—CONR′COR′) imide, (—NR′₂) amino, (—N⁺R′₃) ammonium, (—NR′COR′) amide, (—NR′SO₂R′), sulfonamide (—SO₂R′), sulfonic (—CHO), formyl (—SO₂NR′₂), sulfonamide (—COR′), ketone, in which groups R′ is as follows: C₁-C₅ alkyl, C₁-C₅ perfluoroalkyl, C₅-C₂₄ aryl, C₅-C₂₄ perfluoroaryl, C₇-C₂₄ aralkyl;
wherein the metathesis reaction is optionally carried out in the presence of other additives enhancing the process of the reaction.

22. A method according to claim 21 characterised in that compound 1a is used as compound 1. 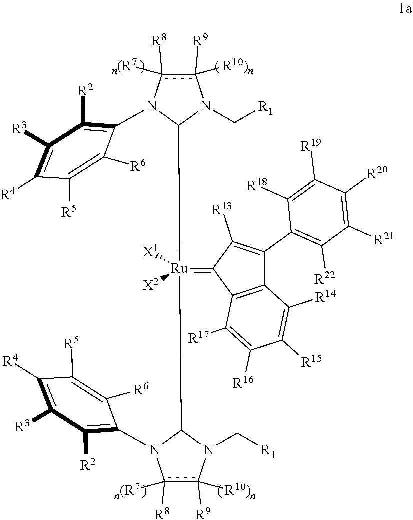
wherein:
X¹, X² are a halogen atom;
R¹ is a heteroaryl selected from the group comprising furan, thiophene, benzothiophene, benzofuran;
R², R³, R⁴, R⁵, R⁶ are each independently a hydrogen atom, methyl, isopropyl, a halogen atom;
R⁷, R¹, R⁹, R¹⁰ are each independently a hydrogen atom or methyl;
n is 0 or 1;
R¹³, R¹⁴, R¹⁵, R¹⁶, R¹⁷, R¹⁸, R¹⁹, R²⁰, R²¹ and R²² are each independently a hydrogen atom, a halogen atom, one of the following groups: C₁-C₂₅ alkyl, C₁-C₂₅ alkylamino, C₁-C₂₅ alkylammonium, C₁-C₂₅ perfluoroalkyl, C₂-C₂₅ alkenyl, C₃-C₇ cycloalkyl, C₃-C₂₅ cycloalkenyl, C₂-C₂₅ alkynyl, C₃-C₂₅ cycloalkynyl, C₁-C₂₅ alkoxy, C₅-C₂₄ aryl, C₅-C₂₀ heteroaryl, C₃-C₁₂ heterocycle, 3-12-membered heterocycle, a sulfide (—SR′), ester (—COOR′), amido (—CONR′₂), sulfonic (—SO₂R′), sulfonamide (—SO₂NR′₂) or ketone (—COR′), group, wherein R′ is a C₁-C₅ alkyl, C₁-C₅ perfluoroalkyl, C₅-C₂₅ aryl or C₅-C₂₅ perfluoroaryl group.

23. A method according to claim 21 characterised in that compound 1b is used as compound 1. 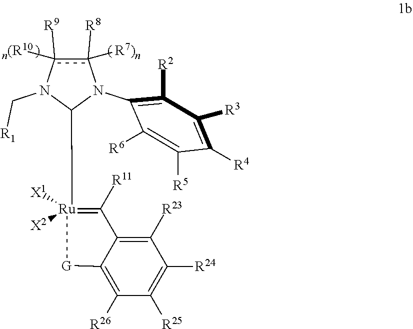
wherein:
X¹, X² are a halogen atom;
R¹ is a heteroaryl selected from the group comprising furan, thiophene, benzothiophene, benzofuran;
R², R³, R⁴, R⁵, R⁶ are each independently a hydrogen atom, methyl, isopropyl, a halogen atom;
R⁷, R¹, R⁹, R¹⁰ are each independently a hydrogen atom or methyl;
n is 0 or 1;
R¹¹ is a hydrogen atom;
R²³, R²⁴, R²⁵, R²⁶ are each independently a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₅-C₂₄ aryl, C₇-C₂₄ aralkyl, C₅-C₂₄ perfluoroaryl, C₅-C₂₀ heteroaryl, 3-12 membered heterocycle, one of the following groups: alkoxy (—OR′), sulfide (—SR′), nitro (—NO₂), cyano (—CN), carboxy (—COOH), ester (—COOR′), amido (—CONR′₂), imido (—CONR′COR′), amino (—NR′₂), ammonium (—N⁺R′₃), amide (—NR′COR′), sulfonamide (—NR′SO₂R′), sulfonate (—SO₂R′), formyl (—CHO), sulfonamide (—SO₂NR′₂), ketone (—COR′), in which R′ groups are as follows: C₁-C₅ alkyl, C₁-C₅ perfluoroalkyl, C₅-C₂₄ aryl, C₅-C₂₄ perfluoroaryl, C₇-C₂₄ aralkyl, wherein R²³, R²⁴, R²⁵, R²⁶ are preferably a hydrogen atom;
G is a halogen atom or a substituent selected from the group OR′, SR′, S(O)R′, S(O)₂R′N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom.

24. A method according to claim 21 characterised in that compound 1c is used as compound 1. 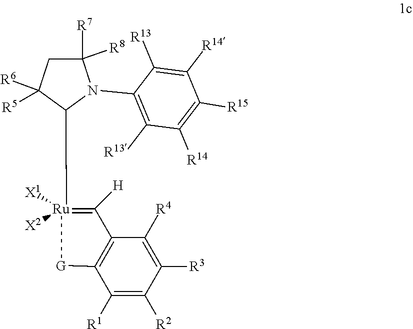
wherein:
X¹, X² are a halogen atom;
G is a halogen atom or a substituent selected from the group OR′, SR′, S(O)R′, S(O)₂R′N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;
R¹, R², R³, R⁴ are each independently a hydrogen atom, a sulfoxide group (—S(O)R′), a sulfonamide group (—SO₂NR′₂), a nitro group (—NO₂), an ester group (—COOR′), a ketone group (—COR′), a —NC(O)R′ ammonium group, a (—OMe) alkoxy group, in which groups R′ is C₁-C₅ alkyl, C₁-C₅ perfluoroalkyl, C₅-C₂₄ aryl, C₇-C₂₄ aralkyl, C₅-C₂₄ perfluoroaryl;
R⁵, R⁶, R⁷, i R⁸ are each independently a hydrogen atom or a C₁-C₂₅ alkyl group, a C₅-C₂₀ aryl group, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom; moreover, R⁵ and R⁶ and/or R⁷ and R may be interconnected to form a cyclic system R¹³ and R^{13′} are each independently methyl or ethyl;
R¹⁴, R^{14′}, R¹⁵ are each independently a hydrogen atom, a C₁-C₂₅ alkyl group.

25. A method according to claim 21 characterised in that compound 1d is used as compound 1. 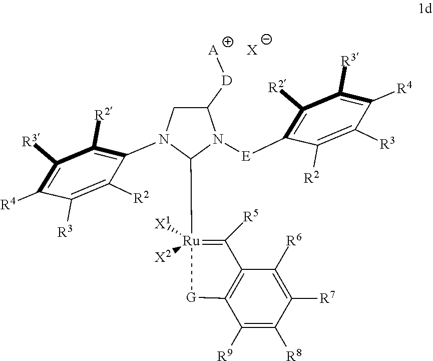
wherein:
the combination A⁺X⁻ and D is a hydrogen atom, or
A is independently a substituent containing a tertiary amine group or a quaternary ammonium group, which may be a N(R¹)(R²) or N⁺(R¹)(R²)(R³) group, wherein R¹, R² and R³ are each independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl; alternatively, A is one of the following groups: C₁-C₂₅ cycloaminoalkyl, C₁-C₂₅ cyclodiaminoalkyl, C₁-C₂₅ cyclotriaminoalkyl, C₁-C₂₅ cyclotetraaminoalkyl, C₁-C₂₅ cycloaminoammonioalkyl, wherein the at least one nitrogen atom is independently substituted with at least one R¹ group, wherein R¹ is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl so that at least one nitrogen atom in the ring forms a quaternary ammonium group;
X is independently a halogen atom, or CF₃SO₃⁻, BF₄⁻, PF₆⁻, and ClO₄⁻;
D is independently one of the following groups: C₁-C₂₅ alkyl, C₁-C₁₂ perfluoroalkyl, C₃-C₇ cycloalkyl, C₁-C₂₅ alkoxy, C₅-C₂₀ aryl, C₅-C₂₄ aryloxy, C₅-C₂₄ perfluoroaryl, C₅-C₂₀ heteroaryl, or C₂-C₂₅ alkenyl, C₁-C₂₅ α,ω-dialkoxy, (CH₂CH₂O)ₙ, polyether, where n comprises from 1 to 25, a C₁-C₂₅ thioalkyl group, a C₁-C₂₅ α,ω-dithioalkyl group, a C₁-C₂₅ α,ω-diheteroalkyl group, a C₁-C₂₅ aminoalkyl group;
E is independently a C₁-C₂₅ alkyl group, a C₁-C₁₂ perfluoroalkyl group, a C₃-C₇ cycloalkyl group, a C₁-C₂₅ alkoxy group, or a single bond;
X¹ and X² are each independently an anion ligand selected from such as halogen atoms;
R¹ is independently a C₁-C₂₅ alkyl group, a C₃-C₇ cycloalkyl group, a C₅-C₂₄ aryl group, a C₅-C₂₀ heteroaryl group;
R², R^{2′}, R³, R^{3′} i R⁴ are each independently a hydrogen atom, a halogen atom, a C₁-C₂₅ alkyl group, a C₃-C₇ cycloalkyl group, a C₁-C₂₅ alkoxy group, a C₅-C₂₄ perfluoroaryl group, a C₅-C₂₀ heteroaryl group or a C₂-C₂₅ alkenyl group, wherein the substituents R², R^{2′}, R³, R^{3′} and R⁴ may be interconnected to form a substituted or unsubstituted cyclic C₄-C₁₀ or polycyclic C₄-C₁₂ system;
R⁵ is a hydrogen atom, a C₁-C₂₅ alkyl group, a C₃-C₇ cycloalkyl group;
R⁶, R⁷, R⁸, R⁹ are each independently a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₅-C₂₄ aryl, C₇-C₂₄ aralkyl, C₅-C₂₄ perfluoroaryl, C₅-C₂₀ heteroaryl, 3-12 membered heterocycle, one of the following groups: alkoxy (—OR′), sulfide (—SR′), nitro (—NO₂), cyano (—CN), carboxy (—COOH), ester (—COOR′), amido (—CONR′₂), imido (—CONR′COR′), amino (—NR′₂), ammonium (—N⁺R′₃), amide (—NR′COR′), sulfonamide (—NR′SO₂R′), sulfonate (—SO₂R′), formyl (—CHO), sulfonamide (—SO₂NR′₂), ketone (—COR′), in which R′ groups are as follows: C₁-C₅ alkyl, C₁-C₅ perfluoroalkyl, C₅-C₂₄ aryl, C₅-C₂₄ perfluoroaryl, C₇-C₂₄ aralkyl, wherein R²³, R²⁴, R²⁵, R²⁶ are preferably a hydrogen atom;
G is a halogen atom or a substituent selected from the group OR′, SR′, S(O)R′, S(O)₂R′N(R′)(R″), P(R′)(R″), wherein R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl group, C₅-C₂₀ aryl group, C₅-C₂₄ aryloxy group, C₂-C₂₀ heterocyclic group, C₄-C₂₀ heteroaryl group, C₅-C₂₀ heteroaryloxy group, or which may be interconnected to form a substituted or unsubstituted cyclic C₄-C₁₀ or polycyclic C₄-C₁₂ system, which are optionally substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy, which can also be substituted with an ester (—COOR′), amide (—CONR′₂), formyl (—CHO), ketone (—COR′), hydroxamic (—CON(OR′)(R′)) groups, wherein R′ is a C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₀ aryl, C₅-C₂₄ aryloxy, C₇-C₂₄ aralkyl, C₂-C₂₀ heterocycle, C₄-C₂₀ heteroaryl, C₅-C₂₀ heteroaryloxy, or a halogen atom;

26. A method according to claim 21 characterised in that compound if is used as compound 1. 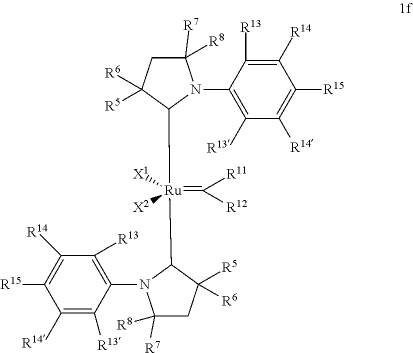
wherein:
X¹ and X² are each independently an anionic ligand selected from such as halogen atoms, —CN, —SCN, —OR′, —SR′, —O(C═O)R′, —O(SO₂)R′, —OSi(R′)₃ group, wherein R′ is C₁-C₁₂ alkyl, C₃-C₁₂ cycloalkyl, C₂-C₁₂ alkenyl, C₅-C₂₀ aryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy, or a halogen atom;
R⁵, R⁶, R⁷, and R⁸ are each independently a hydrogen atom or a C₁-C₂₅ alkyl group, R⁷ and/or R⁸ may be interconnected with R⁹ and/or R¹⁰ to form a cyclic system, they also may be independently the following groups: C₁-C₁₂ alkyl, C₃-C₁₂ cycloalkyl, C₂-C₁₂ alkenyl C₅-C₂₀ aryl, C₁-C₅ perfluoralkyl, C₇-C₂₄ aralkyl, C₅-C₂₄ perfluoroaryl, which are optionally substituted with at least one C₁-C₁₂ alkyl, C₁-C₁₂ perfluoroalkyl, C₁-C₁₂ alkoxy, C₅-C₂₄ aryloxy, C₅-C₂₀ heteroaryloxy or a halogen atom;
R¹¹, R¹² are each independently a hydrogen atom, a halogen atom, optionally substituted C₁-C₂₅ alkyl, optionally substituted C₁-C₂₅ perfluoralkyl, optionally substituted C₂-C₂₅ alkene, optionally substituted C₃-C₇ cycloalkyl, optionally substituted C₂-C₂₅ alkenyl, optionally substituted C₃-C₂₅ cycloalkenyl, optionally substituted C₂-C₂₅ alkinyl, optionally substituted C₃-C₂₅ cycloalkinyl, optionally substituted C₁-C₂₅ alkoxy, optionally substituted C₅-C₂₄ aryloxy, optionally substituted C₅-C₂₀ heteroaryloxy, optionally substituted C₅-C₂₄ aryl, optionally substituted C₅-C₂₀ heteroaryl, optionally substituted C₇-C₂₄ aralkyl, optionally substituted C₅-C₂₄ perfluoroaryl, optionally substituted 3-12-membered heterocycle;
wherein the substituents R¹¹ and R¹² may be interconnected to form a ring selected from the group consisting of C₃-C₇ cycloalkyl, C₃-C₂₅ cycloalkenyl, C₃-C₂₅ cycloalkinyl, C₅-C₂₄ aryl, C₅-C₂₀ heteroaryl, C₅-C₂₄ perfluoroaryl, 3-12-membered heterocycle, each of which may be substituted with one or more substituents selected from the group comprising a hydrogen atom, a halogen atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkinyl, C₃-C₂₅ cycloalkinyl, C₁-C₂₅ alkoxy, C₅-C₂₄ aryloxy, C₅-C₂₀ heteroaryloxy, C₅-C₂₄ aryl, C₅-C₂₀ heteroaryl, C₇-C₂₄ aralkyl, C₅-C₂₄ perfluoroaryl, 3-12-membered heterocycle;
R¹³ and R^{13′} are each independently methyl or ethyl;
R¹⁴, R^{14′}, R¹⁵ are each independently a hydrogen atom, a C₁-C₂₅ alkyl group.

27. A method according to claim 21, characterised in that a compound selected from such as those below is used as compound 1.

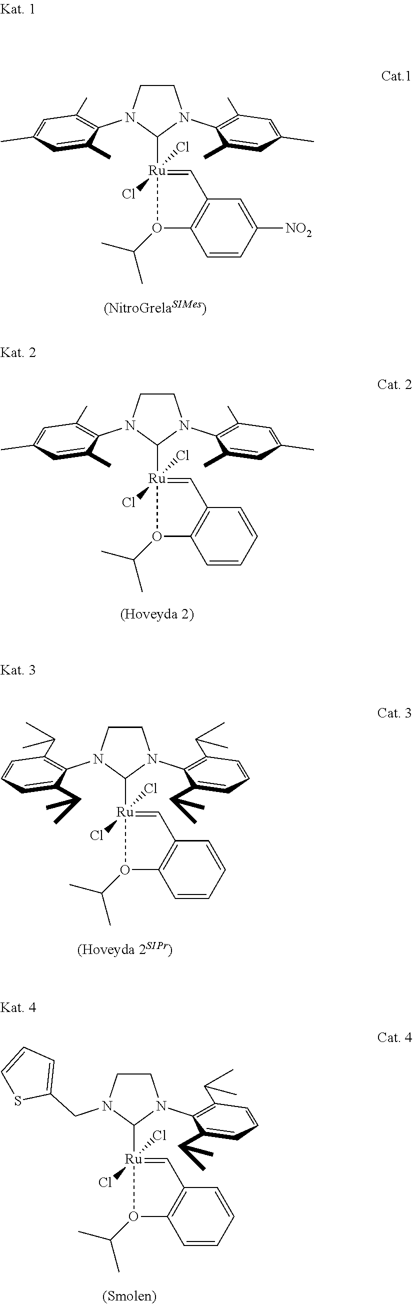

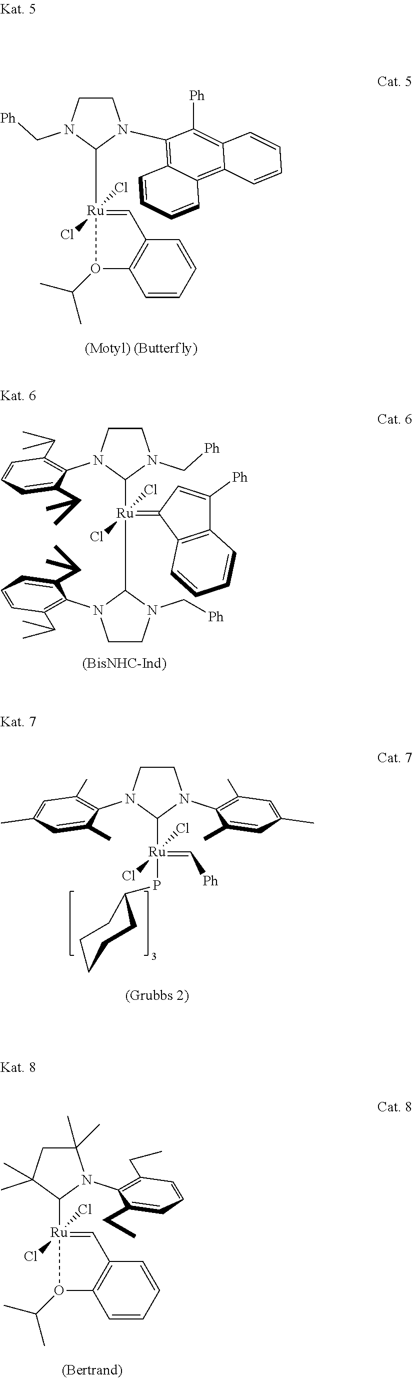

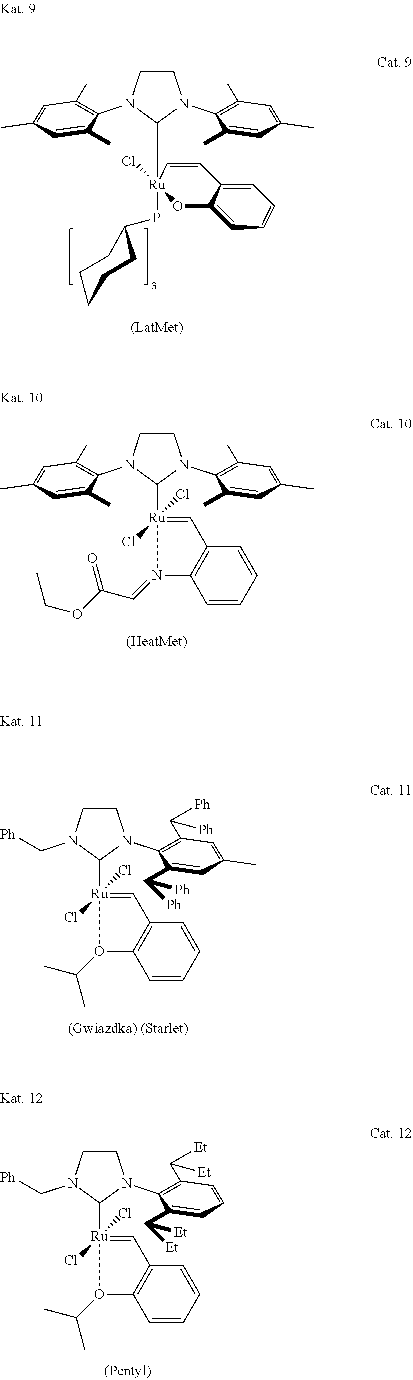

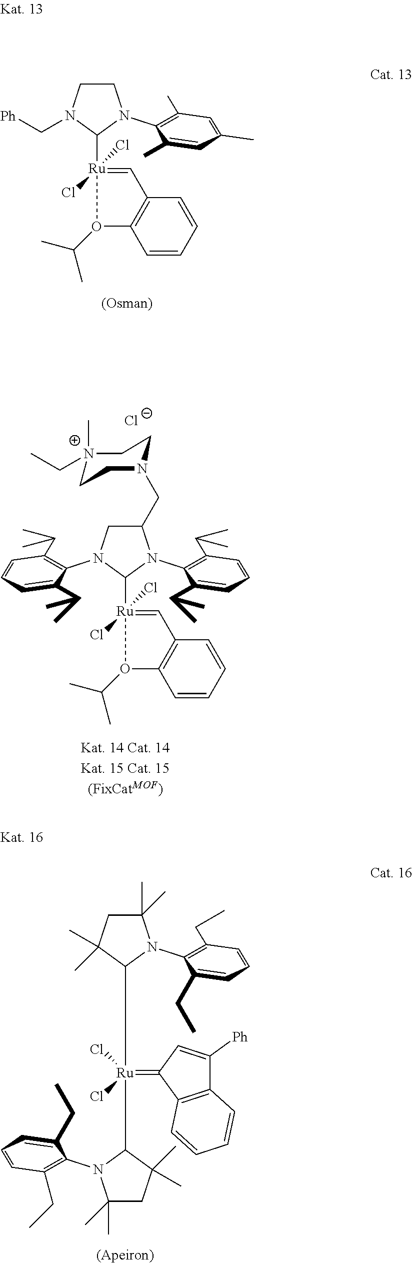

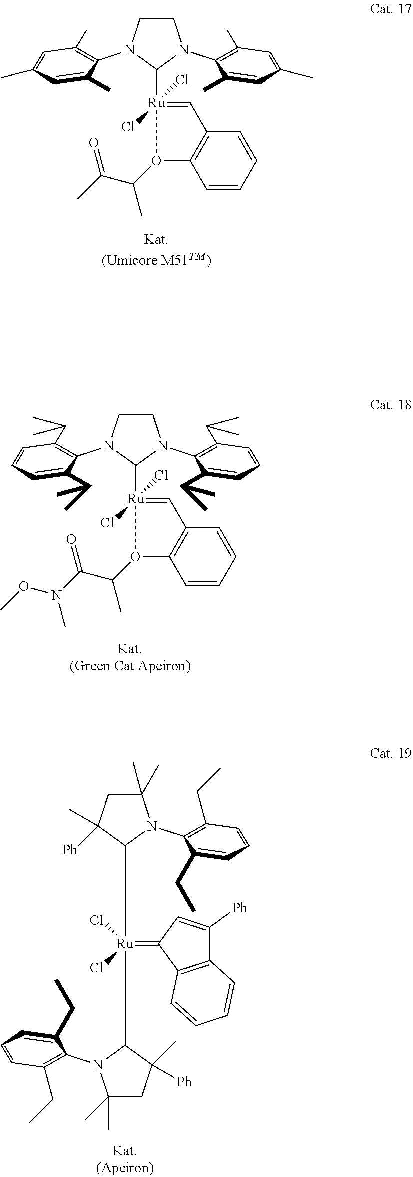

28. The use according to claim 21, characterised in that compound 1 is used deposited on a solid support selected from the group comprising silica gel (SiO₂), aluminium oxide (Al₂O₃), zeolites, celite, or MOF (Metal Organic Framework)-like materials, i.e. potentially porous coordination polymers.

29. The use according to claim 21, characterised in that quinone derivatives are used as an additive in an amount from 5 mol % to 0.05 mol %, preferably such as quinone, anthraquinone, tetrafluoroquinone, tetrachloroquinone and the like.

30. The use according to claim 21, characterised in that the reaction is conducted in an organic solvent such as toluene, benzene, mesitylene, dichloromethane, ethyl acetate, methyl acetate, tert-butyl methyl ether, cyclopentyl methyl ether, paraffin oil, paraffin wax, ionic liquid, polyethylene, PAO polyalphaolefins, preferably PAO 6 and PAO 4, or without any solvent.

31. A method according to claim 21, characterised in that the reaction is conducted with olefin Dy at a concentration of between 1 mM and 1 M.

32. The use according to claim 21, characterised in that the reaction is conducted at a temperature between 20 and 200° C.

33. The use according to claim 21, characterised in that the reaction is conducted for between 5 minutes and 24 hours.

34. The use according to claim 21, characterised in that compound 1 is used in an amount between 2 mol % and 0.0005 mol %.

35. The use according to claim 21, characterised in that compound 1 is added to the reaction mixture in portions and/or continuously using a pump.

36. The use according to claim 21, characterised in that compound 1 is added to the reaction mixture as a solid and/or as a solution in an organic solvent.

37. The use according to claim 21, characterised in that olefin Dy is added to the reaction mixture in portions and/or continuously using a pump.

38. The use according to claim 21, characterised in that the reaction product that is gaseous in the reaction conditions is actively removed from the reaction mixture using inert gas or vacuum.

39. The use according to claim 21, characterised in that the reaction is conducted at a pressure below atmospheric pressure.

40. The use according to claim 21, characterised in that the reaction is conducted at a pressure of between 1 bar and 1·10⁻⁶ mbar.
